(19) United States
(12) Patent Application Publication
(10) Pub. No.: US 2012/0027725 A1
(43) Pub. Date: Feb. 2, 2012

(54) SAFE LENTIVIRAL VECTORS FOR TARGETED DELIVERY OF MULTIPLE THERAPEUTIC MOLECULES TO TREAT LIVER CANCER

(76) Inventors: Jeffrey A. Galvin, Redwood City, CA (US); Zhennan Lai, North Potomac, MD (US); Qiang Yang, Rockville, MD (US)

(21) Appl. No.: 12/957,178

(22) Filed: Nov. 30, 2010

Related U.S. Application Data
(60) Provisional application No. 61/265,199, filed on Nov. 30, 2009.

(51) Int. Cl. A61K 35/76; A61P 35/00; C12N 15/63

(52) U.S. Cl. 424/93.2; 435/320.1

(57) ABSTRACT

The present application discloses a lentiviral transfer system for treating liver cancer which includes: (i) a self-inactivating transfer vector comprising: multiple gene units, wherein each gene unit includes a heterologous nucleic acid sequence operably linked to a regulatory nucleic acid sequence; and (ii) a helper construct which lacks a 5′ LTR, wherein the 5′ LTR has been replaced with a heterologous promoter, in which the helper construct further comprises: a lentiviral env nucleic acid sequence containing a deletion, wherein the deleted env nucleic acid sequence does not produce functional env protein; and a packaging signal contains a deletion, wherein the deleted packaging signal is nonfunctional.

### BACKGROUND OF THE INVENTION

1. Field of the Invention

The present invention relates to the field of medicine, specifically to delivery of multiple therapeutic molecules strategically combined with regulatory elements, using a safe for human use, highly and long-term expressing in human subject lentiviral gene transfer vector for the treatment of liver cancer.

2. General Background and State of the Art

The present application relates to gene transfer vectors that provides versatility, control, high expression, stable multiple gene expression, tolerability, and safety, for the design of effective gene therapies for liver cancer.

Hepatocellular carcinoma (HCC) is the major form of liver cancer, and the fifth most common cancer in the world. In the United States, an estimated 24,120 new cases will be diagnosed in 2010. An estimated 18,910 will die from the disease in 2010. Globally, an estimated 711,000 new cases were diagnosed in 2007, with an estimated 680,000 deaths.

HCC is most often associated with a pre-existing insult that results in chronic liver injury. Sources of insult seen in the United states that have been shown to precede HCC include infection with hepatitis B virus (HBV) or hepatitis C virus (HCV) and cirrhosis of the liver, usually from excessive alcohol consumption. The chronic injury resulting from these causes seems to drive repeated cycles of hepatocyte death and liver regeneration. It is thought that repeated cycles of cell division in this situation increases the likelihood of oncogenic changes in hepatocytes. Often the latency between insult and malignancy can span 20 to 30 years.

Early stage HCC is relatively asymptomatic, therefore, it is often diagnosed incidentally. Most often patients are diagnosed with late stage disease. Overall 1-year and 3-year survival rates are estimated to be approximately 36% and 17%, respectively.

Identification of target genes involved in neoplastic transformation and tumor progression has encouraged the idea that nucleotide sequences of cancer-relevant genes could lead to the development of tailored anticancer agents that lack many of the toxic side effects of traditional cytotoxic drugs. Mutations of the p53 gene are associated with transformation to a malignant phenotype. Transfer of wild-type P53, which plays a critical role in the regulation of cell growth and downregulation of genes that contribute to cancer progression, is hoped to result in selective and specific inhibition of tumor growth while minimizing undesirable side effects on normal cells. Delivery of the p53 gene has been reported in a replication-deficient adenoviral vector containing the wild-type p53 gene sequence. Functional activity and expression of the transgene product in tumor cells treated with the adenoviral vector has been reported. (Baker, et al., 1990, Suppression of human colorectal carcinoma cell growth by wild-type p53, Science 249: 912-915.) Two adenovirus-based gene therapeutics for the treatment of cancer were recently commercialized in China. These two agents, combined with chemotherapy, have been used there as an alternative treatment for some types of refractory cancer. (Peng, 2005, Current Status of Gendicine in China: Recombinant Human Ad-p53 Agent for Treatment of Cancers, Hum. Gene Ther. 16, 1016-1027; Yu, W., and Fang, H., 2007, Clinical Trials with Oncolytic Adenovirus in China. Curr. Cancer Drug Targets 7, 141-148). Although adenovirus vectors can efficiently deliver therapeutic genes in both dividing and non-dividing cells, and can be manufactured at high viral titers, adenovirus vectors are highly immunogenic (Shirakawa, et al., 2008, The Current Status of Adenovirus-based Cancer Gene Therapy, Mol. Cells 25(4): 462-466). Furthermore, long-term expression in target tissues is not observed.

Lentiviruses, such as HIV, are “slow viruses.” Vectors derived from lentiviruses can be expressed long-term in the host cells after a few administrations to the patients, e.g., via ex vivo transduced bone marrow stem cells. For most diseases and disorders, including genetic diseases, cancer, and neurological disease, long-term expression is crucial to successful treatment. Safety has been a concern with lentiviral vectors, but a number of strategies for eliminating the ability of lentiviral vectors to replicate have now been described. For example, the deletion of promoter and enhancer elements from the U3 region of the long terminal repeat (LTR) are thought to have no LTR-directed transcription. The resulting vectors are called “self-inactivating” (SIN). However, it has been reported that HIV-1-derived vectors containing the SIN deletion in the U3 region of the LTR are capable of expressing full-length genomic transcripts (Logan, et al., 2004, Integrated Self-Inactivating Lentiviral Vectors Produce Full-Length Genomic Transcripts Competent for Encapsidation and Integration, J. Virology 78(16): 8421-8436). Therefore, combination of this deletion with other safety measures must be considered.

The last few years have seen immense excitement regarding the use of RNAi agents for disease therapies. Investigators reported that they were able to specifically silence mutant oncogenic ras without affecting wild-type ras in vitro (Zhang, et al., 1995, Safety evaluation of AdSCMV-p53 in vitro and in vivo, Human Gene Therapy 6:155-164; Ishii, et al., 2001, Potential cancer therapy with the fragile histidine triad gene review of the preclinical studies, JAMA 286: 2441-2449). It is believed that treatment costs for siRNA would be similar to most protein-based therapies, e.g., antibody therapies. Preclinical cancer studies have shown inhibition of growth and survival of tumor cells by RNAi-mediated downregulation of several key oncogenes or tumor-promoting genes, including growth and angiogenic factors or their receptors (vascular endothelial growth factor, epidermal growth factor receptor), human telomerase (hTR, hTERT), viral oncogenes (papillomavirus E6 and E7) or translocated oncogenes (BCR-abl).

Various studies report on the in vivo activity and the potential of RNAi agents to suppress tumor growth. These include an intratumoral injection of an shRNA-adenoviral vector construct targeting a cell-cycle regulator causing inhibition of subcutaneous small cell lung tumor in mice, and systemic administration of an siRNA targeting a carcinoembryonic antigen-related cell adhesion molecule (CEACAM6) in mice with subcutaneously xenografted pancreatic adenocarcinoma cells. In another report, direct injection of a plasmid vector expressing shRNAs to matrix metalloproteinase MMP-9 and a cathepsin showed efficacy in established glioblastoma (Chen, et al., 2005, Reversal of the phenotype by K-rasval12 silencing mediated by adenovirus-delivered siRNA in human pancreatic cancer cell line Panc-1, World J. Gastroenterol. 11(6): 831-838). However, delivery of siRNA for long-term expression in target cells and tissues has been particularly difficult in vivo.

Another problem in gene transfer is the delivery of therapeutic molecules to a sufficient number of target cells to elicit a therapeutic response. Recently, a series of virus-encoded and other regulatory proteins were found to possess the ability to cross biological membranes. These proteins include HIV-Tat and the herpes simplex virus type 1 tegument protein VP22. VP22 was also reported to exhibit a unique property of effecting intercellular spread. VP22 is a basic, 38-kDa phosphorylated protein (Knopf, et al., 1980, J. Gen. Virol. 46:405-414) encoded by the viral UL49 gene (Elliott, et al., 1992, J. Gen. Virol. 73:723-726).

Specific and controlled delivery of therapeutic molecules to an affected cell population, e.g., to tumor cells and even circulating cancer cells, can potentially be achieved by strategically positioning nucleic acid and protein regulatory elements, e.g., cell and tissue-specific promoters and enzyme cleavage sites. These elements are recognized by the production machinery that is present only in certain cell types. The ability to easily combine and regulate the expression and delivery of multiple therapeutic molecules, while taking effective safety measures without compromising expression levels, in methods for using a lentiviral gene transfer vector or lentiviral transfer system, would provide a researcher with a critical tool for treating a broad range of diseases and disorders.

### SUMMARY OF THE INVENTION

The present invention is related to lentiviral transfer systems including safe, self-inactivating, recombinant lentiviral vectors with the capacity to accommodate strategic combinations of genes for therapeutic molecules and novel regulatory sequences, in methods for treating a broad range of diseases and disorders.

In one aspect, the invention relates to a lentiviral gene transfer system comprising: a self-inactivating transfer vector comprising: a first gene unit with a first heterologous nucleic acid sequence, operably linked to a first regulatory nucleic acid sequence; and a second gene unit with a second heterologous nucleic acid sequence, operably linked to a second regulatory nucleic acid sequence; and a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce a functional env protein; a packaging signal containing a deletion, wherein said deleted packaging signal is nonfunctional. The transfer vector may be preferably derived from HIV-1. Preferably, RNAi or a polypeptide may be encoded by the heterologous nucleic acid sequence. In addition, expression of the first and second heterologous nucleic acid sequences may have a synergistic effect in inhibiting progression of a disease or disorder.

The transfer vector may include mammalian insulator sequence and splice acceptor and splice donor sites, and may be free of wPRE “wood-chuck” hepatitis virus post-transcriptional element downstream of a cloning site (Gao et al., J. Virol., March 2008; p. 2938-2951).

The RNAi may inhibit expression of a gene that contributes to progression of a disease or disorder. In addition, the first or second heterologous nucleic acid sequence may include a sequence encoding a trafficking signal, and the trafficking signal may be expressed as a fusion with a protein expressed from the second heterologus nucleic acid sequence. The intercellular trafficking signal may be a membrane-penetrating protein or a fragment thereof, such as a plant or bacterial protein toxin, or viral protein, any other sequence domain with transporting function between cells, in particular, cancer cells. The trafficking signal may be derived from herpesvirus VP22 or HIV-Tat, or may be a HIV-Tat eleven amino acid transduction sequence. The herpesvirus may be HSV1, and the trafficking signal may further be a VP22 protein homologue of HSV1 VP22. The VP22 transport signal may include a C-terminal 34 amino acid sequence of VP22 of HSV1, or a fragment having 80% or greater identity to the terminal 34 amino acid sequence of VP22 of HSV1. Further, the VP22 transport signal may include one or more of RSASR, RTASR, RSRAR, RTRAR, ATATR, or RSAASR.

The transfer vector may utilize general or cell or tissue specific promoters such as TSTA promoter, mesothelin promoter, hPSA promoter, hCCKAR promoter, hAFP promoter, and hNSE promoter.

Tissue-specific enzyme cleavage sites may be included in the transfer vector, wherein cleavage at the site occurs within a polypeptide that is encoded by the first and second heterologous nucleic acid sequences. The regulatory nucleic acid sequence may include a sequence encoding a cell or tissue-specific enzyme cleavage site, wherein cleavage at the site occurs within at least one polypeptide that is encoded by two or more of the first, second and third heterologous nucleic acid sequences. The cell or tissue-specific enzyme cleavage site may be a protease 2A cleavage site, a presecretory protein signal peptidase cleavage site, or a pancreatic prechymotrypsinogen cleavage site.

The transfer vector may also include a nucleic acid sequence encoding translation initiation site. Such a sequence may be positioned between the gene units, and in certain aspects may be considered to belong to a “regulatory sequence” of a gene unit. Although a variety of sequences may be used, an internal ribosome entry site (IRES) is preferred.

The inventive lentiviral transfer system may be used to prepare a treatment for a disease or disorder. The disease or disorder may be cancer. In a preferred embodiment, a heterologous nucleic acid sequence may encode the P53 protein.

The antisense RNA, RNAi, or any polypeptide expressed from the heterologous nucleic acid sequence may be expressed consistently and for long period of time to inhibit expression of a gene or the activity of a gene product that contributes to progression of the cancer. The antisense RNA, RNAi and the polypeptide may inhibit expression and activity of a tumor promoting gene or gene product. The RNAi and the expressed polypeptide may inhibit expression of or activity of a growth factor, growth factor receptor, angiogenic factor, angiogenic factor receptor, cell cycle regulator, apoptosis-inducing molecule, or cell adhesion molecule. The RNAi or the expressed polypeptide may inhibit the expression or activity of a vascular endothelial growth factor, Bcl-2, K-ras, AEG-1, Myc, including c-Myc, a vascular endothelial growth factor receptor, epidermal growth factor receptor, hTR, hTERT, papillomavirus E6, papillomavirus E7, BCR-abl, CEACAM6, MMP9, or a cathepsin.

The cancer to be treated may be prostate cancer in which a regulatory nucleic acid sequence may include hPSA. If the cancer is liver cancer then a regulatory nucleic acid sequence may include hAFP. If the cancer is pancreatic cancer then a regulatory nucleic acid sequence may include hCCKAR to control the expression of RNAi or a polypeptide.

The inventive lentiviral transfer system may be used to prepare a treatment for a genetic disorder, such as a metabolic disorder, including Gaucher's Disease or Fabry's Disease. In the case of Gaucher's Disease, a first heterologous nucleic acid sequence may encode glucocerebrosidase and a second heterologous nucleic acid sequence may encode a human intrinsic selectable marker such as huCD25 protein or huNGF protein.

The lentiviral transfer system may include a regulatory nucleic acid sequence that includes a sequence encoding a trafficking signal that is expressed as a fusion with a glucocerebrosidase protein expressed from an adjacent heterologous nucleic acid sequence. A second regulatory nucleic acid sequence may include translation initiation sequence as well. A cell or tissue-specific enzyme cleavage site may also be included.

In the case of Fabry's Disease, said first heterologous nucleic acid sequence may encode an alpha-galactosidase-A protein, and a second heterologous nucleic acid sequence may encode the huCD25 protein.

In another aspect, the inventive lentiviral transfer system may include a first heterologous nucleic acid sequence comprising a sequence encoding a trafficking signal that is expressed as a fusion with the alpha-galactosidase-A protein expressed from the first heterologous nucleic acid sequence. The trafficking signal may be a VP22 trafficking signal or an HIV-Tat trafficking signal.

In yet another aspect, if the genetic disorder is Leber Congenital Amaurosis, the first heterologous nucleic acid sequence may encode the RPE65 protein, and a second heterologous nucleic acid sequence may encode the hBDNF protein, and a second regulatory nucleic acid sequence may include hNSE, and further the vector may include a third heterologous nucleic acid sequence encoding the hNGF protein, and also a third regulatory nucleic acid sequence including a sequence encoding a cell or tissue-specific enzyme cleavage site.

In another aspect, the disease or disorder to be treated may be a neurological disorder, such as Alzheimer's Disease, in which case, a first heterologous nucleic acid may encode the hNGF protein, a second heterologous nucleic acid may encode an RNAi targeted to beta-amyloid precursor protein, and wherein the second regulatory nucleic acid sequence may include a sequence encoding a cell or tissue-specific enzyme cleavage site. A third third heterologous nucleic acid that encodes the hBDNF protein may also be included, in which the first regulatory nucleic acid sequence may include a sequence encoding a trafficking signal that is expressed as a fusion with the hNGF protein expressed from the first heterologous nucleic acid sequence.

If the neurological disorder is Parkinson's Disease, a first heterologous nucleic acid may encode the hBDNF protein or the hGDNF protein, and the second heterologous nucleic acid encodes hGAD. The second regulatory nucleic acid sequence may include a sequence encoding a cell or tissue-specific enzyme cleavage site. Further, the first regulatory nucleic acid may include a sequence encoding a trafficking signal that is expressed as a fusion with the hBDNF protein or hGDNF protein expressed from the first heterologous nucleic acid sequence, wherein the vector may further include a third heterologous nucleic acid sequence encoding hNGF, in which the vector may further include a third regulatory nucleic acid sequence encoding a cell or tissue-specific enzyme cleavage site.

In another aspect, the invention is directed to a method for treating a condition, comprising administering to a patient a lentiviral particle for gene transfer, said lentiviral particle produced using a lentiviral transfer system comprising: a self-inactivating transfer vector comprising: a first gene unit with a first heterologous nucleic acid sequence, operably linked to a first regulatory nucleic acid sequence; and a second gene unit with a second heterologous nucleic acid sequence, operably linked to a second regulatory nucleic acid sequence; and a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional. The condition may be cancer, such as liver cancer, pancreatic cancer, or prostate cancer. The condition may also be a genetic disorder, such as Gaucher's Disease or Fabry's Disease. The condition may also be a neurological disorder, such as Parkinson's Disease or Alzheimer's Disease. The condition may also be a need for cosmetic enhancement.

A pharmaceutical composition comprising a lentiviral particle for gene transfer, said lentiviral particle produced using a lentiviral transfer system comprising: a self-inactivating transfer vector comprising: a first gene unit with a first heterologous nucleic acid sequence, operably linked to a first regulatory nucleic acid sequence; and a second gene unit with a second heterologous nucleic acid sequence, operably linked to a second regulatory nucleic acid sequence; and a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional.

In a further aspect, the invention is directed to a pharmaceutical composition that includes a lentiviral transfer vector, said lentiviral transfer vector comprising a first heterologous nucleic acid sequence, operably linked to a first regulatory nucleic acid sequence; and a second heterologous nucleic acid sequence, operably linked to a second regulatory nucleic acid sequence, wherein said transfer vector is self-inactivating.

The pharmaceutical compositions as described above may further include a chemotherapeutic agent, a steroid agent such as prednisolone, cortisone, corticosterone, or dexamethasone.

In one aspect of the invention, SIN element is incorporated into the recombinant lentivirus; the tat region has been modified so as to optionally allow for infection efficiency without allowing the replication functions and uncontrolled infection of the recombinant lentivirus beyond the intended target; and the rev protein has been inactivated so as to prevent further unwanted infectivity while preserving the basic function of the rev to support the expression efficiency of the therapeutic gene(s). Tat and rev proteins may be inactivated of their original replication activity without necessarily removing them from the lentivirus and thereby preserve their desirable attributes while keeping the resulting vector bio-safe.

The present application is also directed to the following subject matter:

In one aspect, the invention is directed to a lentiviral transfer system comprising:

(i) a self-inactivating transfer vector comprising: multiple gene units, wherein each gene unit comprises a heterologous nucleic acid sequence operably linked to a regulatory nucleic acid sequence, wherein one or more of the regulatory nucleic acid sequence comprises a liver cell or liver tissue-specific promoter; and

(ii) a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional.

The tissue-specific promoter may bee hAFP promoter or liver-selective phosphoenolpyruvate carboxykinase promoter and optionally may include a Transcription Amplification system. The polypeptide or RNAi inhibits expression or activity of B cell leukemia/lymphoma-2 (Bcl-2), myc oncogene or astrocyte elevated gene-1 (AEG-1).

The polypeptide, anti-sense RNA or RNAi may inhibit expression or activity of a growth factor, growth factor receptor, angiogenic factor, angiogenic factor receptor, cell cycle regulator, apoptosis-inducing molecule, or cell adhesion molecule. The polypeptide, anti-sense RNA or RNAi may inhibit the expression or activity of a vascular endothelial growth factor, a vascular endothelial growth factor receptor, epidermal growth factor receptor, hTR, hTERT, papillomavirus E6, papillomavirus E7, BCR-abl, CEACAM6, MMP9, AEG-1, Bcl-2, C-Myc, M2 subunit of ribonucleotide reductase (RRM2), or a cathepsin.

In another aspect, the invention is directed to a method for treating liver cancer, comprising administering to a patient a lentiviral particle for gene transfer, said lentiviral particle produced using a lentiviral transfer system comprising:

(i) a transfer vector comprising: multiple gene units, wherein each gene unit comprises a heterologous nucleic acid sequence operably linked to a regulatory nucleic acid sequence, wherein one or more of the regulatory nucleic acid sequence comprises a liver cell or liver tissue-specific promoter; and

(ii) a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional.

The cell or tissue-specific promoter is hAFP promoter or liver-selective phosphoenolpyruvate carboxykinase promoter and optionally may include Transcription Amplification system. The polypeptide or RNAi may inhibit expression or activity of B cell leukemia/lymphoma-2 (Bcl-2), myc oncogene or astrocyte elevated gene-1 (AEG-1), and express p53.

In another aspect, the invention is directed to A pharmaceutical composition comprising a lentiviral particle for gene transfer, said lentiviral particle produced using a lentiviral transfer system comprising:

(i) a self-inactivating transfer vector comprising: multiple gene units, wherein each gene unit comprises a heterologous nucleic acid sequence operably linked to a regulatory nucleic acid sequence, wherein one or more of the regulatory nucleic acid sequence comprises a liver cell or liver tissue-specific promoter; and

(ii) a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional. The heterologous nucleic acid sequence may encode a P53 protein and an antisense RNA or RNAi is targeted for B cell leukemia/lymphoma-2 (Bcl-2), myc oncogene or astrocyte elevated gene-1 (AEG-1). The cell or tissue-specific promoter may be hAFP promoter or liver-selective phosphoenolpyruvate carboxykinase promoter and may include optionally Transcription Amplification system. The composition may include a chemotherapeutic agent or a steroid agent, which may include prednisolone, cortisone, corticosterone, or dexamethasone. A pharmaceutical composition may include a self inactivating lentiviral transfer vector comprising multiple gene units, wherein each gene unit comprises a heterologous nucleic acid sequence operably linked to a regulatory nucleic acid sequence.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 Lentiviral transfer system. The drawing shows an HIV-1-based gene transfer system carrying multiple functional (therapeutic) genes. A. Helper (packaging) construct. The 5′ LTR of the helper construct is replaced with the CMV promoter, to avoid integration of the viral elements presented in that construct. This is an important biosafety feature of the lentiviral transfer system of the invention. The triangles represent deletions: one represents a 36-bp deletion harboring the putative packaging signal from nucleotides 753 to 789 between the 5′ major splice donor site and the beginning of the gag ATG coding region; one represents a deletion in the tat gene, and a third represents a deletion in nef. The packaging signal is functionally absent from this construct, to avoid production of an active gag-pol precursor. The poly (A) site was derived from the bovine growth hormone gene. The helper construct provides a nucleic acid sequence encoding lentiviral gag and pol, operably linked to a heterologous regulatory nucleic acid sequence. The construct further contains a deleted, nonfunctional env protein and is devoid of lentiviral sequences both upstream and downstream from a splice donor site to a lentiviral gag initation site. B. Envelope expression construct. An envelope construct encoding vesicular stomatitis virus G glycoprotein (VSV-G) is shown, though other non-lentiviral envelope proteins can be used instead. Expression is driven by the HIV-1 LTR. The poly(A) site was derived from the simian virus 40 late region. C. Transfer vector constructs. In these constructs, Tat, Vpr, and Nef are inactivated. Boxes interrupted by jagged lines contain partial deletions. RRE=Rev-response element; ψ=cis-acting packaging signal; IRES=internal ribosome entry site; huCD25=human IL-2Ra chain gene; GFP=Green Fluorescent Protein coding sequence; RNAi=interfering RNA coding sequence; S=stop codon; T =termination signal (e.g., SV40 polyA or BGH polyA); CS=cleavage site (e.g., viral 2A-like peptide cleaved by the 2A protease, presecretory protein cleavage site, pancreatic prechymotrypsinogen cleavage site); hPSA =human prostate specific antigen promoter; P132 promoter 1; CMV, Human CMV-IE promoter; P2=promoter-2; P53=tumor suppressor gene; dsRNA=Bcl-2 RNAi (human).

FIG. 2 Heterologous Proteins. This table provides examples of heterologous proteins contemplated for use in the vectors and methods of the present invention.

FIG. 3 Transport Genes. This table provides examples of transport genes contemplated for use in developing transport sequences for the vectors and methods of the present invention.

FIG. 4 Promoter Elements. This table provides examples of promoter elements contemplated for use in the vectors and methods of the present invention.

FIG. 5 Enhancer Elements. This table provides examples of enhancer elements contemplated for use in the vectors and methods of the present invention.

FIG. 6 SIN-LV-P53-EGFP and SIN-LV-BCL2 RNAi-EGFP. The drawing shows the vector constructs used as described in the Examples.

FIG. 7 Therapeutic Constructs. This table provides examples of constructs for use in the vectors and methods of the invention for certain therapeutic applications.

FIG. 8 Infection of Prostate Cancer Cells. A. EGFP expression in PC-3 cells infected with SIN-HIV-p53-IRES-EGFP (Panel 1) and PC-3 cells not infected with virus (2). B. EGFP expression in 293T cells infected with SIN-HIV-p53-IRES-EGFP (Panel 1) and 293T cells not infected with virus (2). C. Viral packaging of Vector. D. P53 expression. Lane 1: Size standards 1 kb plus DNA Ladder. Lane 2: PC-3 cells (no infection). Lane 3: PC-3 cells (infection). Lane 4: Blank. Lane 5: 293T cells (no infection). Lane 6: 293T cells (infection).

FIG. 9 Therapeutic Constructs 2. This table provides examples of constructs for use in the vectors and methods of the invention for certain therapeutic applications.

FIGS. 10A-10B Experimental evidence of efficacy of the viral vector construct P53-Bcl-2 RNAi, expressing P53 and an RNAi agent targeting human Bcl-2. FIGS. 10A and 10B show phenotype of in vitro cell culture under phase-contrast microscope indicating that the P53-Bcl-2 RNAi construct induces cell necrosis in PC3 prostate cancer cells. A. untreated living cells. B. treated with viral vector construct P53-Bcl-2 RNAi, expressing P53 and an RNAi agent targeting human Bcl-2.

FIGS. 11A-11B Experimental evidence of efficacy of the viral vector construct P53-Bcl-2 RNAi, expressing P53 and an RNAi agent targeting human Bcl-2. FIGS. 11A and 11B show phenotype of in vitro cell culture under phase-contrast microscope indicating that the P53-Bcl-2 RNAi construct does not cause necrosis in 293T cells. A. untreated cells. B. treated with viral vector construct P53-Bcl-2 RNAi, expressing P53 and an RNAi agent targeting human Bcl-2.

FIG. 12 In vivo test demonstrates tumor reduction efficacy of viral vector construct P53-Bcl-2 RNAi, expressing P53 and an RNAi agent targeting human Bcl-2. In order from left to right, far left bar is weight of the control tumor group after three weeks; tumor treated with P53 alone expressed through viral vector construct (repair genetic defect); tumor treated with BCL2 SiRNA alone expressed through viral vector construct (down regulation of BCL2 gene) only; and on the far right tumor treated with viral vector construct P53-Bcl-2 RNAi, expressing P53 and an RNAi agent targeting human Bcl-2 for simultaneous P53 (repair) and BCL2 siRNA.

FIGS. 13A-13B show in vivo tumor staining of control (untreated tumor) versus treatment groups. Immunohistochemical staining by specific antibodies show that tumor cells treated with viral vector construct P53-Bcl-2 RNAi, expressing P53 and an RNAi agent targeting human Bcl-2 no longer produce hPSA (human prostate specific antigen), which means that the cells are no longer active tumor cells or are no longer actively cancerous. A. control tumor expresses hPSA. B. treatment tumor shows cell necrosis and no expreession of hPSA.

FIGS. 14A-14C show a table that shows results from another mouse (in vivo) study confirming tumor reduction findings. Group V1 is prostrate tumor mice treated with P53 expressed alone in a viral vector; Group V2 is tumor mice treated with BCL2 siRNA alone expressed through viral vector; V3 is tumor mice treated with the dual viral construct expressing P53 and BCL2 siRNA. Group PC shows untreated tumor mice control. “**” in the tables indicates tumor sizes on Day 7 as the starting sizes for calculating the tumor growth rate and the ratio of Tumor Weigh/Starting Tumor Size for groups of V1, V2, V3, and PC.

FIG. 15 shows a graph of combination treatment of JZ2 and JZ3 virus on HCC cell line Hep G2.

FIG. 16 shows dose response of JZ2 and JZ3 virus on HCC cell line Hep G2.

### DETAILED DESCRIPTION OF THE PREFERRED EMBODIMENTS

In the present application, “a” and “an” are used to refer to both single and a plurality of objects.

Unless otherwise indicated, all terms used herein have the same ordinary meaning as they would to one skilled in the art of the present invention.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

As used herein, reference to “upstream”, “downstream”, “first gene”, “second gene”, “last gene”, “before”, “after” and so forth in relation to the spatial positioning of the various DNA sequences in a vector, is meant to be with respect to the 5′ to 3′ orientation of the vector sequence. For example “before” will have the same meaning as “upstream of” or 5′ of a particular reference position, and “after” will have the same meaning of “downstream of” or 3′ with respect to a particular reference point on the vector.

As used herein, “gene unit” includes a regulatory region that may include a promoter and a heterologous nucleic acid sequence encoding either an antisense RNA, RNAi or polypeptide of interest that is controlled by the regulatory sequence, which is typically referred to in the context of a multigene transfer vector. However, in situations where a fused polypeptide is desirous of being generated from the multigene vector of the encoded polypeptides of adjacent gene units, the regulatory region of the downstream gene units may include a nucleic acid sequence encoding a cleavage site instead of a separate promoter.

### Modes of Carrying out the Invention

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

I. Lentiviral Transfer System

The present invention provides a recombinant lentivirus capable of infecting dividing and non-dividing cells. The virus is useful for the in vivo and ex vivo transfer and expression of nucleic acid sequences. Lentiviral vectors of the invention may be lentiviral transfer plasmids or infectious lentiviral particles. Construction of lentiviral vectors, helper constructs, envelope constructs, etc., for use in lentiviral transfer systems has been described, e.g., in U.S. Patent App. Pub. No. 2003/0119770, “Intercellular delivery of a herpes simplex virus VP22 fusion protein from cells infected with lentiviral vectors,” incorporated herein by reference in its entirety.

Lentiviruses

Lentiviruses are RNA viruses wherein the viral genome is RNA. When a host cell is infected with a lentivirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. As described below, a helper virus is not required for the production of the recombinant lentivirus of the present invention, since the sequences for encapsidation are provided by co-transfection with appropriate vectors.

The lentiviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vif, vpr, tat, rev, vpu, nef, and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site, ψ). If the sequences necessary for encapsidation (or packaging of lentiviral RNA into infectious virions) are missing from the viral genome, the result is a cis defect which prevents encapsidation of genomic RNA. The resulting mutant is still capable of directing the synthesis of all virion proteins, but lacks function of replication.

In a first embodiment, the invention provides a recombinant lentivirus capable of infecting a dividing or non-dividing cell. The recombinant lentivirus comprises a nucleic acid sequence containing a lentiviral packaging signal flanked by lentiviral cis-acting nucleic acid sequences necessary for reverse transcription and integration, a heterologous nucleic acid sequence operably linked to a regulatory nucleic acid sequence, and a nucleic acid sequence encoding an intercellular trafficking signal, where the nucleic acid sequence encoding the intercellular trafficking signal is fused in-frame with the heterologous nucleic acid sequence, where the lentivirus does not contain either a complete gag, pol, tat, rev, or env gene.

The recombinant lentivirus of the invention is therefore genetically modified in such a way that some of the structural, infectious genes of the native virus have been removed, and some removed sequences replaced with a nucleic acid sequence to be delivered to a target non-dividing cell. After infection of a cell by the virus, the virus releases its nucleic acid into the cell and the lentivirus genetic material can integrate into the host cell genome. The transferred lentivirus genetic material is then transcribed and translated, e.g., as dictated by the regulatory sequences, into proteins within the host cell.

Lentiviral Vector Systems

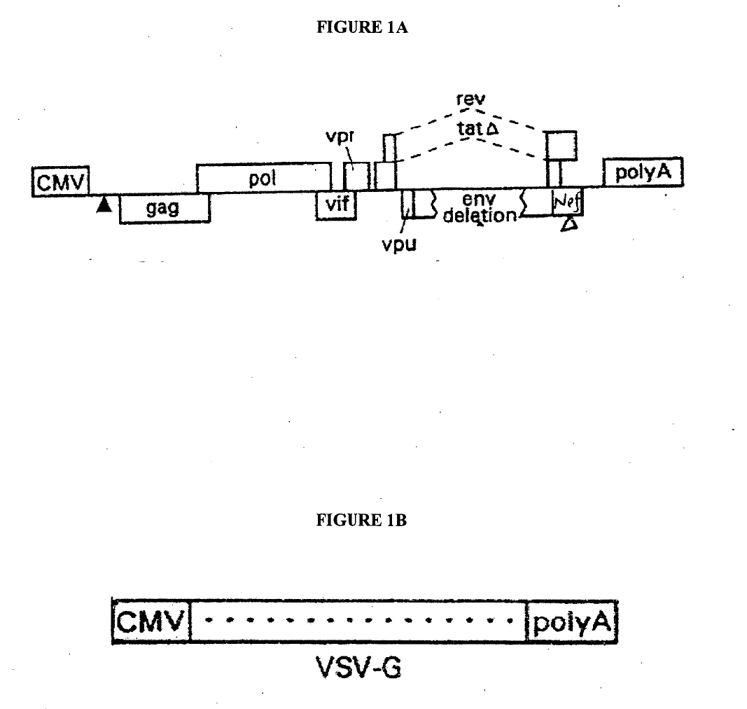

The invention provides a method of producing a recombinant lentivirus capable of infecting a dividing or non-dividing cell comprising transfecting a suitable host cell with the following: a transfer vector providing a nucleic acid encoding a lentiviral gag and a lentiviral pol, where the gag and pol nucleic acid sequences are operably linked to a heterologous regulatory nucleic acid sequence and where the transfer vector is defective for nucleic acid sequence encoding functional env protein and devoid of lentiviral sequences both upstream and downstream from a splice donor site to a gag initiation site of a lentiviral genome; an envelope construct providing a nucleic acid encoding a non-lentiviral env protein; and a helper construct providing a nucleic acid sequence containing a lentiviral packaging signal flanked by lentiviral cis-acting nucleic acid sequences for reverse transcription and integration, and providing a cloning site for introduction of a heterologous nucleic acid sequence operably linked to a regulatory nucleic acid sequence and optionally to a nucleic acid sequence encoding an intercellular trafficking signal, where the nucleic acid sequence encoding the intercellular trafficking signal is fused in-frame with the heterologous nucleic acid sequence, where the helper construct does not contain either a complete gag, pol, or env gene, and recovering the recombinant lentivirus. An illustration of the individual vectors used in the method of the invention is shown in FIG. 1.

The method of the invention includes the combination of a minimum of three vectors in order to produce a recombinant virion or recombinant lentivirus. For example, a vector of the invention can include (a) the p53 gene product, expressed and driven by a regulatory nucleic acid sequence to treat tumor cells or migrating cells having a p53 gene mutation; (b) a specific siRNA driven by second regulatory nucleic acid sequence to down-regulate tumor activity of the tumor cells in target tissue or organs; wherein the double or multiple gene system is able to enhance delivery efficacy and therapeutic response. It is understood that in the vectors and methods of the present invention, the relative positions in the transfer vector of the therapeutic molecules—be they proteins, RNAi or other types of antisense agents—can vary as needed. Therefore, for example, any of the first, second, or third heterologous nucleic acid sequences can encode an RNAi. Furthermore, multiple heterologous nucleic acid sequences (e.g., two or three) can encode an RNAi or other antisense agent.

A first vector is a helper construct, which provides a nucleic acid encoding a lentiviral gag and a lentiviral pol (FIG. 1A).

A second vector is an envelope construct, which provides a nucleic acid encoding a non-lentiviral env protein (FIG. 1B). The env gene can be derived from any virus excluding lentiviruses. The env gene is ideally derived from a virus other than HIV. The env gene may be amphotropic envelope protein which allows transduction of cells of human and other species, or may be ecotropic envelope protein, which is able to transduce only mouse and rat cells. Further, it may be desirable to target the recombinant virus by linkage of the envelope protein with an antibody or a particular ligand for targeting to a receptor of a particular cell-type. By inserting a sequence (including regulatory region) of interest into the viral vector, along with another gene which encodes the ligand for a receptor on a specific target cell, for example, the vector is now target specific. Lentiviral vectors can be made target specific by inserting, for example, a protein. Targeting is often accomplished by using an antibody to target the lentiviral vector. Those of skill in the art will know of, or can readily ascertain without undue experimentation, specific methods to achieve delivery of a lentiviral vector to a specific target.

Examples of retroviral-derived env genes include, but are not limited to: Moloney murine leukemia virus (MoMuLV), Harvey murine sarcoma virus (HaMuSV), murine mammary tumor virus (MuMTV), gibbon ape leukemia virus (GaLV), and Rous Sarcoma Virus (RSV). Other env genes such as Vesicular stomatitis virus (VSV) (Protein G) can also be used.

The construct providing the viral env nucleic acid sequence is operably associated with regulatory sequence, e.g., a promoter or enhancer. Preferably, the regulatory sequence is a viral promoter. The regulatory sequence can be any eukaryotic promoter or enhancer, including for example, the Moloney murine leukemia virus promoter-enhancer element, the human cytomegalovirus enhancer, or the vaccinia P7.5 promoter. In some cases, such as the HIV-1 promoter-enhancer element, these promoter-enhancer elements are located within or adjacent to the LTR sequences.

A third vector, the transfer vector, provides a nucleic acid sequence, which contains the cis-acting viral sequences necessary for the lentiviral life cycle. Such sequences include the lentiviral psi packaging sequence, reverse transcription signals, integration signals, viral promoter, enhancer, and polyadenylation sequences. The transfer vector also contains a cloning site for a heterologous nucleic acid sequence to be transferred to a dividing or non-dividing cell, and optionally a nucleic acid sequence encoding an intercellular trafficking signal, where the nucleic acid sequence encoding the intercellular trafficking signal is fused in-frame with the heterologous nucleic acid sequence (FIG. 1C).

Since recombinant lentiviruses produced by standard methods in the art are defective, they require assistance in order to produce infectious vector particles. Typically, this assistance is provided, for example, by using a helper cell line that provides the missing viral functions. These plasmids are missing a nucleotide sequence which enables the packaging mechanism to recognize an RNA transcript for encapsidation. Suitable cell lines produce empty virions, since no genome is packaged. If a lentiviral vector is introduced into such cells in which the packaging signal is intact, but the structural genes are replaced by other genes of interest, the vector can be packaged and vector virion produced.

Conveniently during the cloning stage, the nucleic acid construct referred to as the transfer vector, having the packaging signal and the heterologous cloning site, also contains a selectable marker gene. Marker genes are utilized to assay for the presence of the vector, and thus, to confirm infection and integration. Typical selection genes encode proteins that confer resistance to antibiotics and other toxic substances, e.g. histidinol, puromycin, hygromycin, neomycin, methotrexate, etc.

“Non-dividing” cell refers to a cell that does not go through mitosis. Non-dividing cells may be blocked at any point in the cell cycle, (e.g., G₀/G₁, G₁/S, G₂/M), as long as the cell is not actively dividing. For ex vivo infection, a dividing cell can be treated to block cell division by standard techniques used by those of skill in the art, including, irradiation, aphidocolin treatment, serum starvation, and contact inhibition. However, it should be understood that ex vivo infection is often performed without blocking the cells since many cells are already arrested (e.g., stem cells). The recombinant lentivirus vector of the invention is capable of infecting any non-dividing cell, regardless of the mechanism used to block cell division or the point in the cell cycle at which the cell is blocked. Examples of pre-existing non-dividing cells in the body include neuronal, muscle, liver, skin, heart, lung, and bone marrow cells, and their derivatives.

In another embodiment, the invention provides a recombinant lentivirus produced by the method of the invention as described above.

The invention also provides a method of nucleic acid transfer to a non-dividing cell to provide expression of a particular nucleic acid sequence. Therefore, in another embodiment, the invention provides a method for introduction and expression of a heterologous nucleic acid sequence in a non-dividing cell comprising infecting the non-dividing cell with the recombinant virus of the invention and expressing the heterologous nucleic acid sequence in the non-dividing cell.

It may be desirable to modulate the expression of a gene regulating molecule in a cell by the introduction of a molecule by the method of the invention. The term “modulate” envisions the suppression of expression of a gene when it is over-expressed, or augmentation of expression when it is under-expressed. Where a cell proliferative disorder is associated with the expression of a gene, nucleic acid sequences that interfere with the gene's expression at the translational level can be used. This approach utilizes, for example, antisense nucleic acid, ribozymes, or triplex agents, siRNA to block transcription or translation of a specific mRNA, either by masking that mRNA with an antisense nucleic acid or triplex agent, or by cleaving it with a ribozyme.

The method of the invention may also be useful for neuronal or glial cell transplantation, or “grafting,” which involves transplantation of cells infected with the recombinant lentivirus of the invention ex vivo, or infection in vivo into the central nervous system or into the ventricular cavities or subdurally onto the surface of a host brain. Such methods for grafting will be known to those skilled in the art and are described in Neural Grafting in the Mammalian CNS, Bjorklund and Stenevi, eds. (1985). Procedures include intraparenchymal transplantation, (i.e., within the host brain) achieved by injection or deposition of tissue within the host brain so as to be apposed to the brain parenchyma at the time of transplantation.

Self-Inactivating Lentiviral Vectors

Self-inactivating (SIN) lentiviral vectors have a deletion in the U3 region of the 3′ LTR that eliminates regulatory sequences, including the TATA box. The deletion has been reported to result in transcriptional inactivation of the LTR in proviruses without affecting vector titers or transgene expression in vitro. SIN vectors are described, e.g., by Zufferey, et al., 1998, J. Virology 72(12):9873-9880, who made a 400 bp deletion, and Miyoshi, et al., 1998, J. Virology 72(10):8150-8157, who made a 133 bp deletion.

It has been reported that a certain U3 deletion actually results in increased expression from the vector in vivo (Bayer, et al., 2008, A Large U3 Deletion Causes Increased In Vivo Expression from a Nonintegrating Lentiviral Vector, Molecular Therapy doi:10.1038/mt.2008.199). This finding suggests that additional alterations to the lentivirus sequences are needed to ensure safety of gene transfer systems.

II. Heterologous Nucleic Acid Sequences

A heterologous nucleic acid sequence is operably linked to a regulatory nucleic acid sequence. As used herein, the term “heterologous” nucleic acid sequence refers to a sequence that originates from a foreign species, or, if from the same species, it may be substantially modified from its original form. Alternatively, an unchanged nucleic acid sequence that is not normally expressed in a cell is a heterologous nucleic acid sequence. The term “operably linked” refers to functional linkage between the regulatory sequence and the heterologous nucleic acid sequence. The heterologous sequence can be linked to a promoter. The heterologous nucleic acid sequence can be under control of either the viral LTR promoter-enhancer signals or of an internal promoter, and retained signals within the lentiviral LTR can still bring about efficient integration of the vector into the host cell genome. The use of nonintegrating vectors for certain purposes, e.g., where transient expression is sufficient, is also contemplated.

The recombinant virus of the invention is capable of transferring nucleic acid sequences into a non-dividing cell. The term “nucleic acid sequence” refers to any nucleic acid molecule, preferably DNA. The nucleic acid molecule may be derived from a variety of sources, including DNA, cDNA, synthetic DNA, RNA, or combinations thereof. Such nucleic acid sequences may comprise genomic DNA which may or may not include naturally occurring introns. Moreover, such genomic DNA may be obtained in association with promoter regions, introns, or poly(A) sequences. Genomic DNA may be extracted and purified from suitable cells by means well known in the art. Alternatively, messenger RNA (mRNA) can be isolated from cells and used to produce cDNA by reverse transcription or other means.

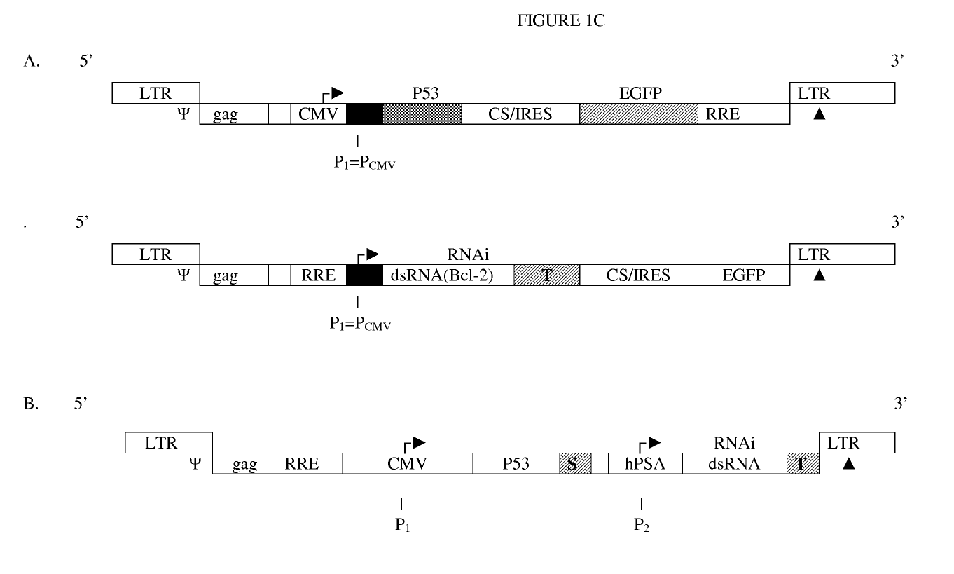

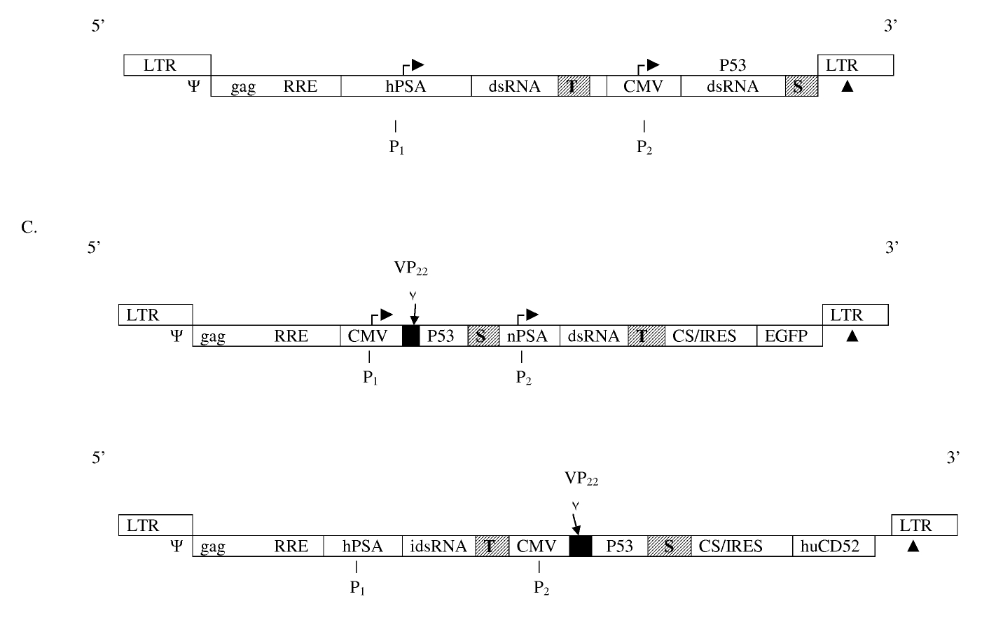

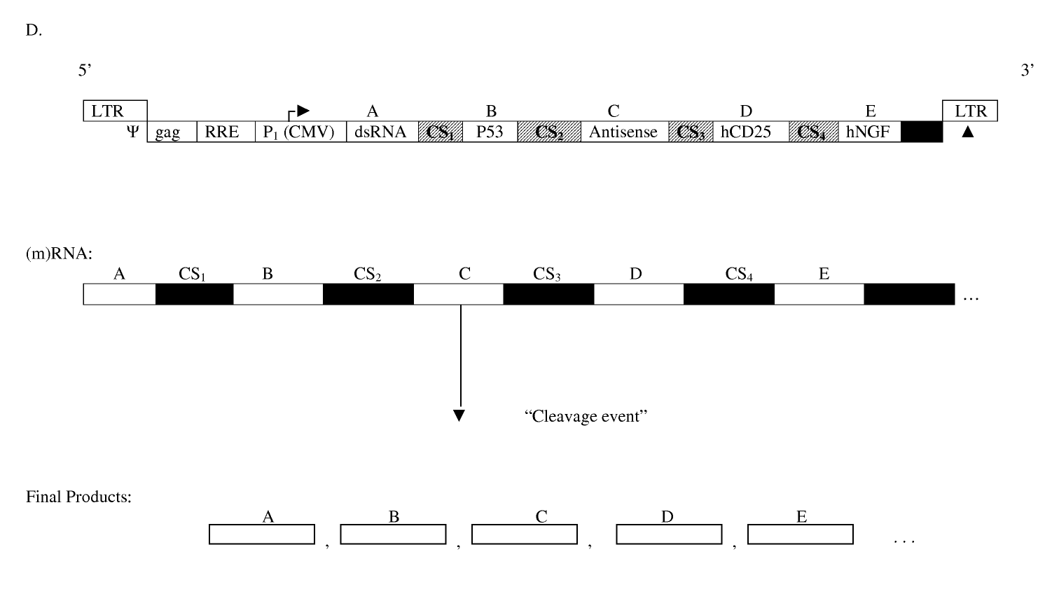

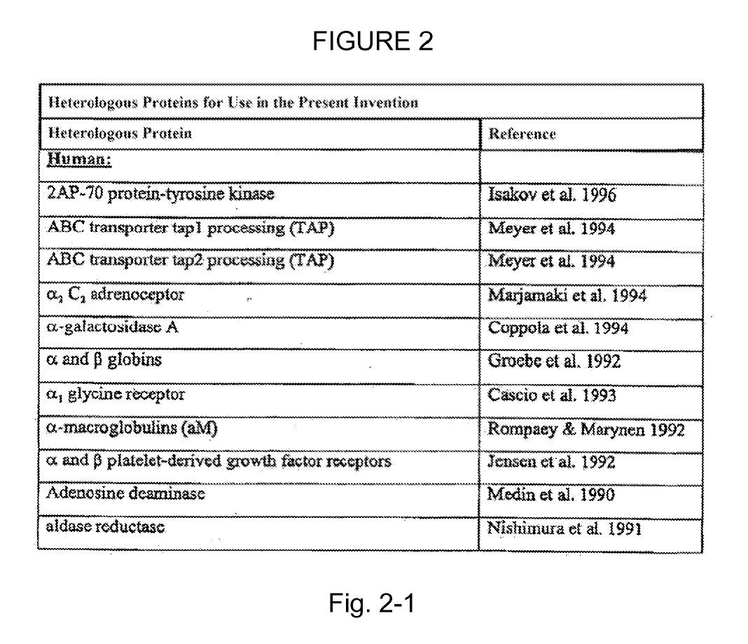

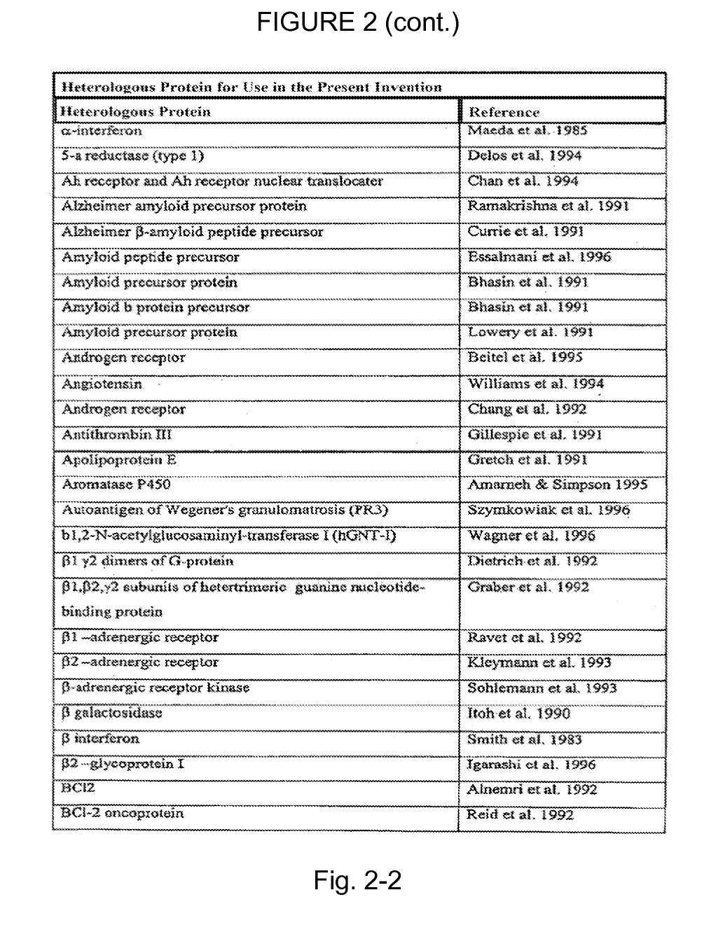

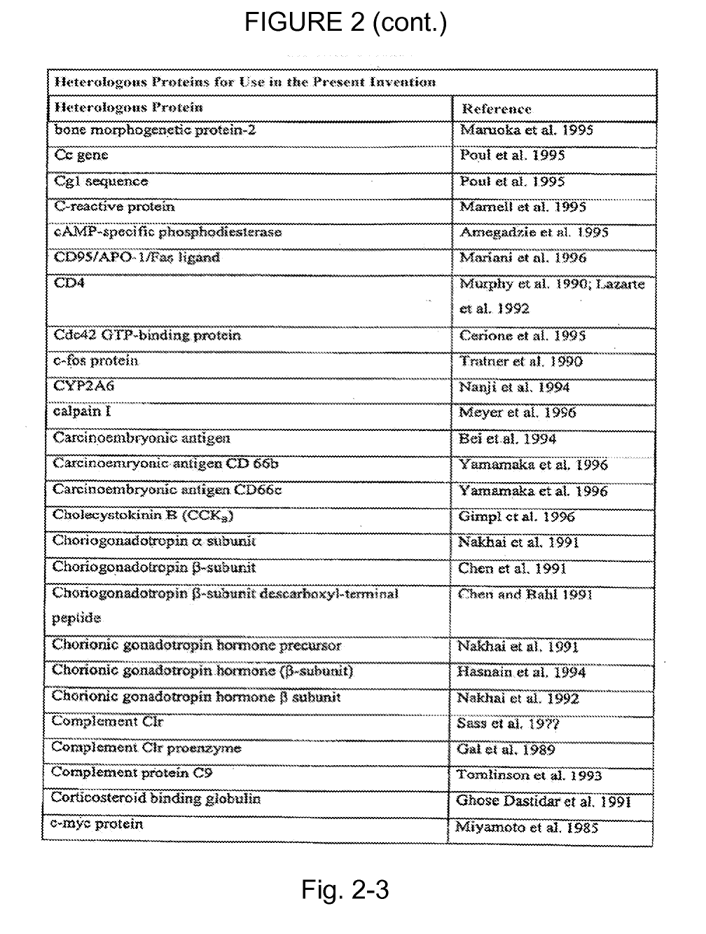

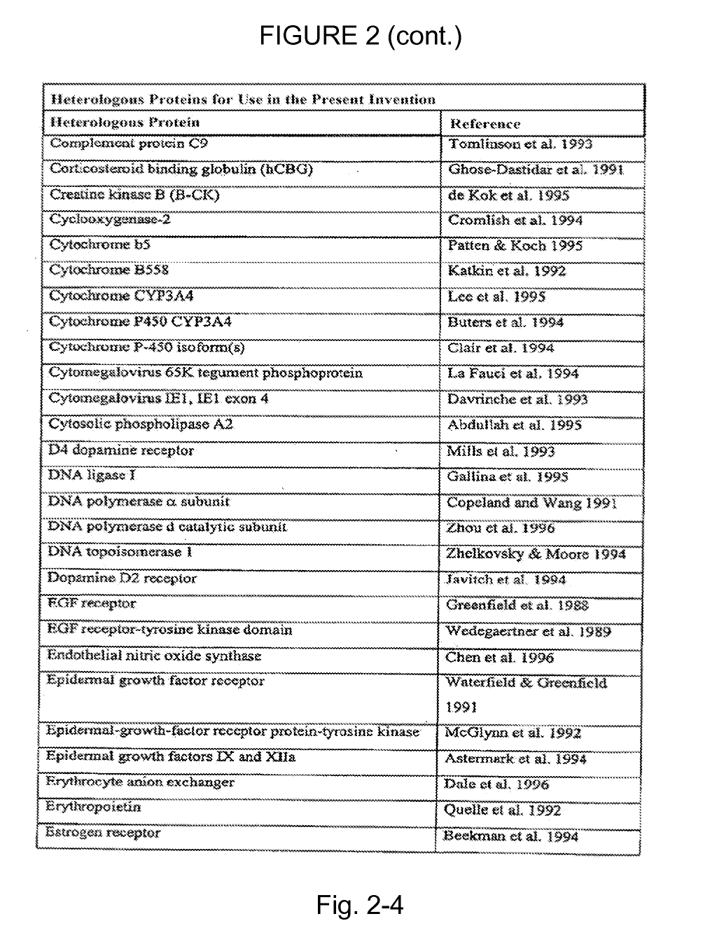

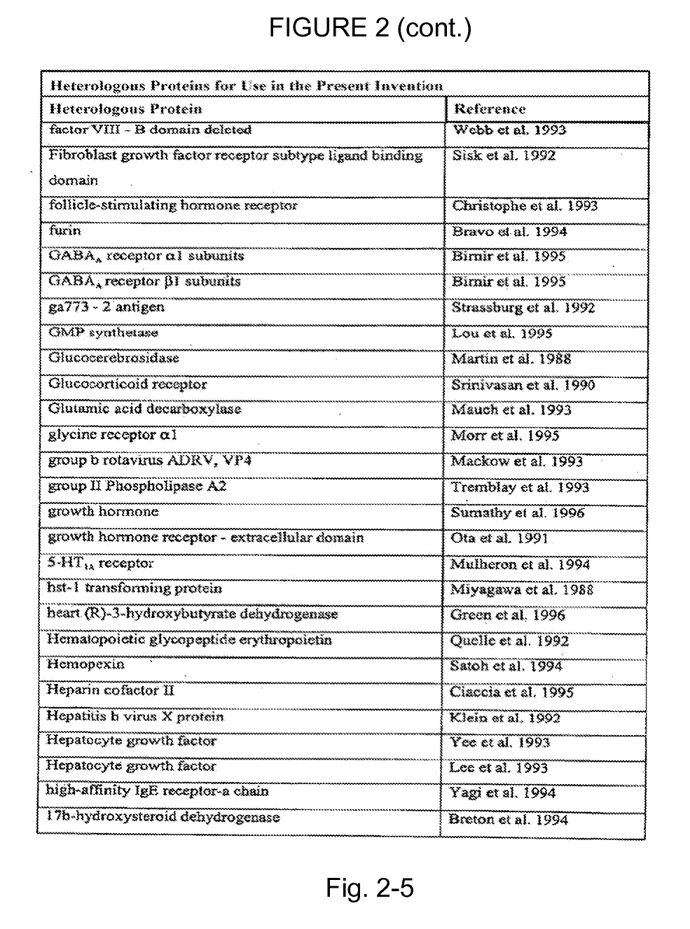

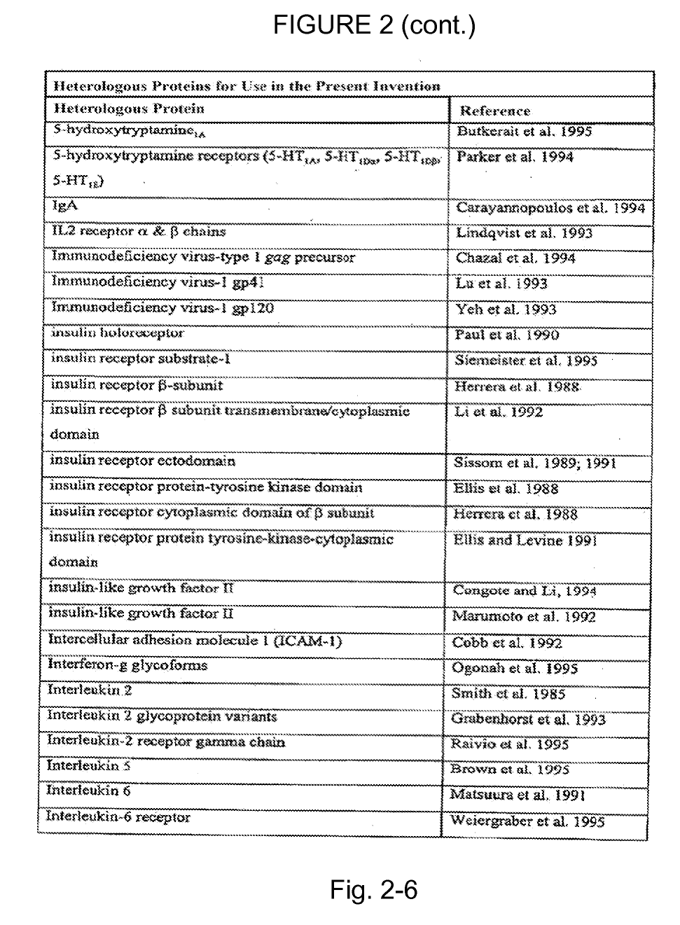

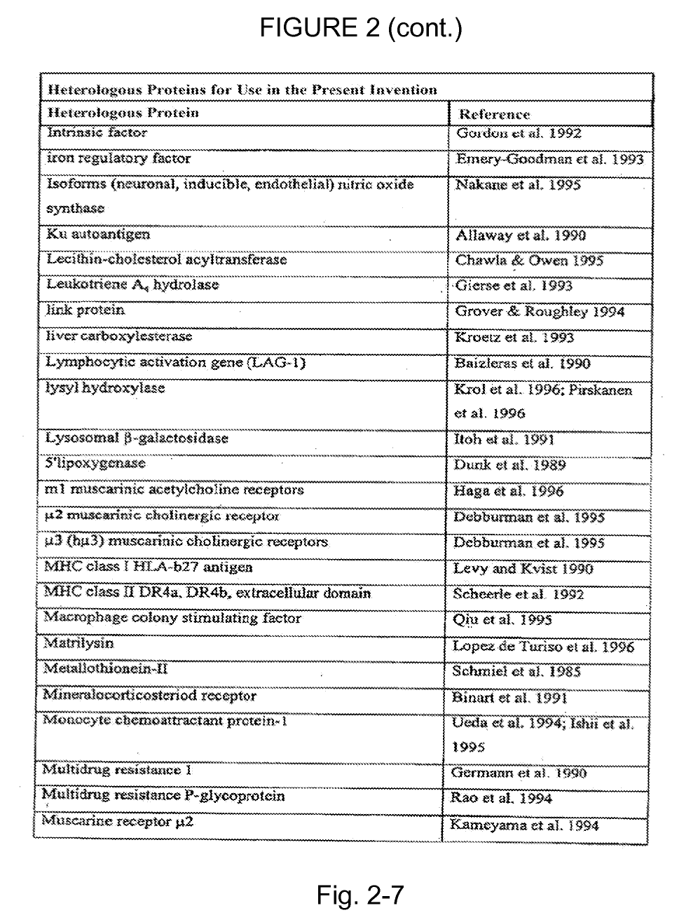

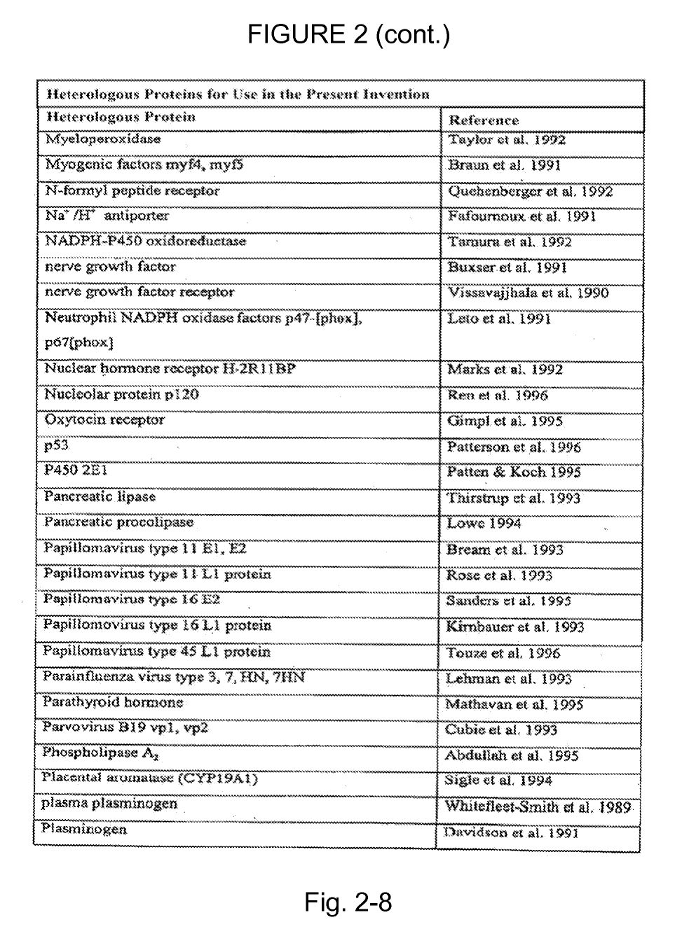

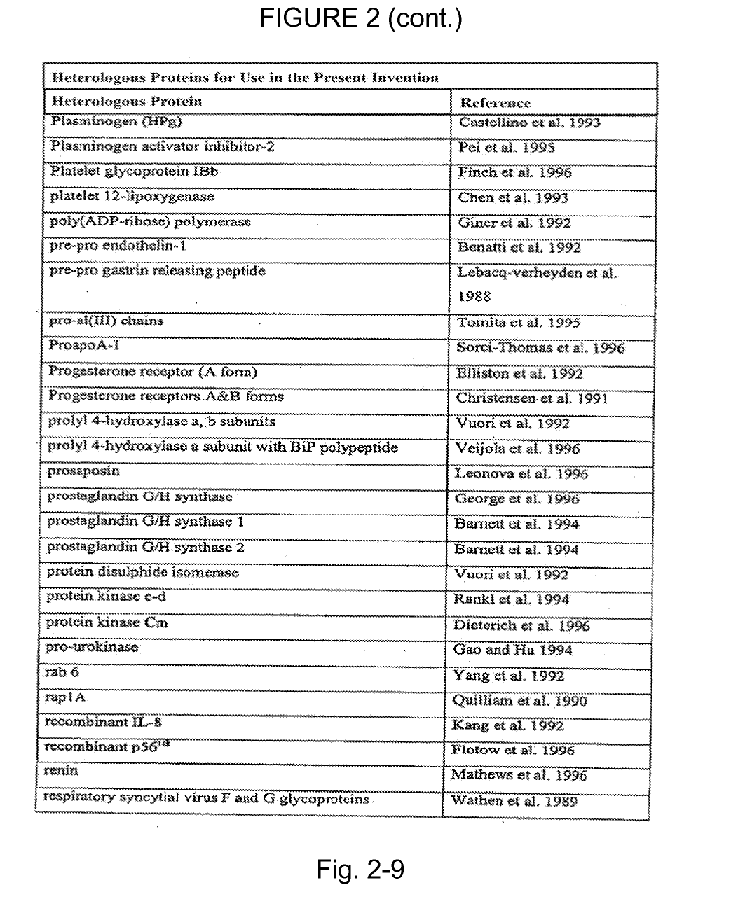

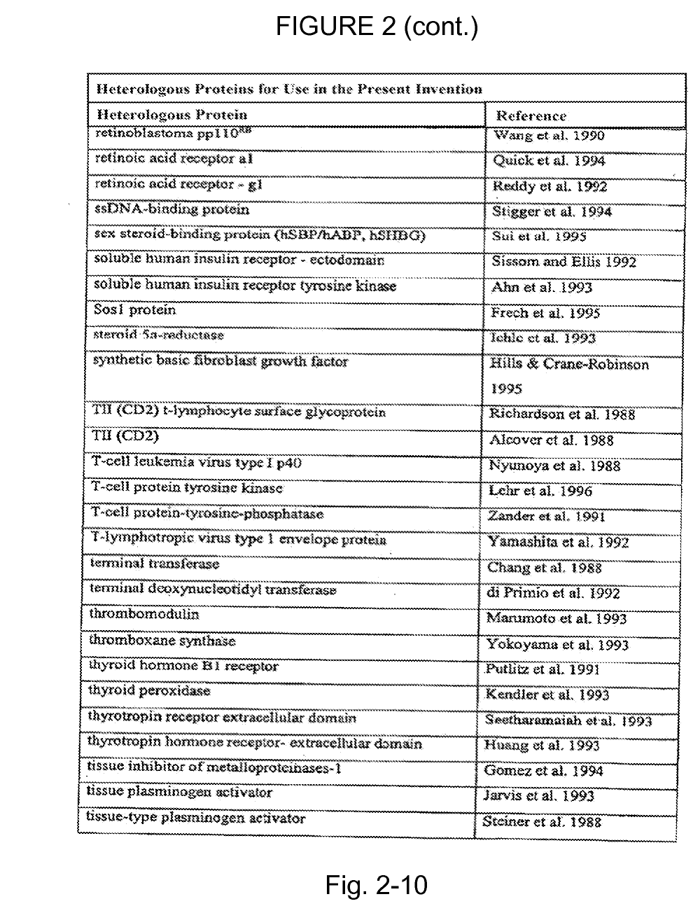

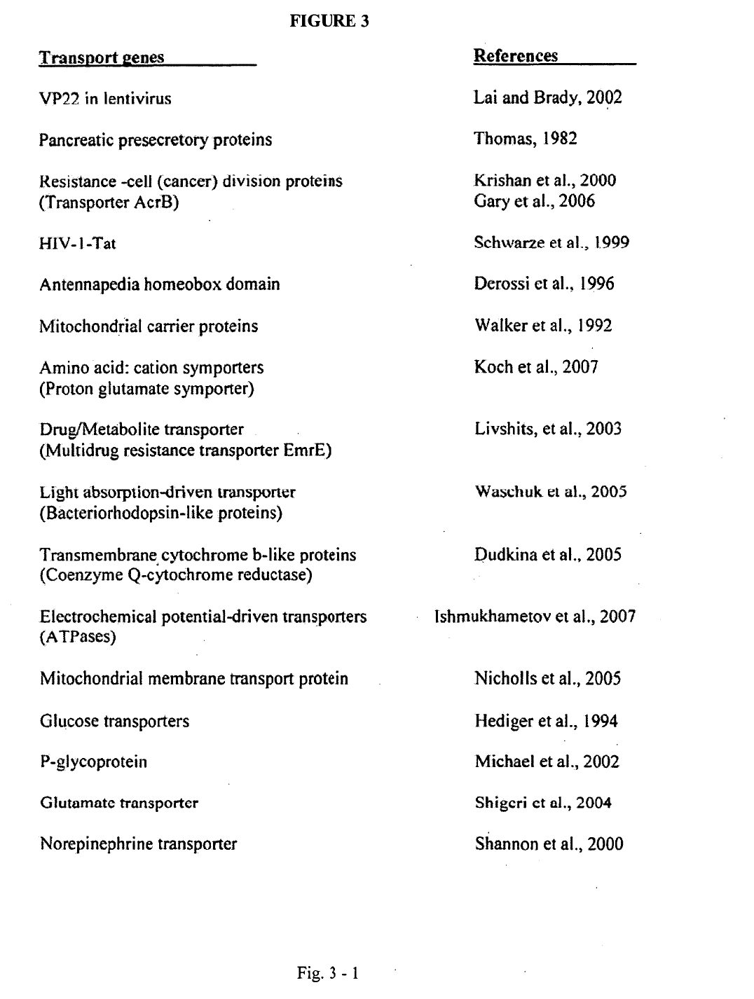

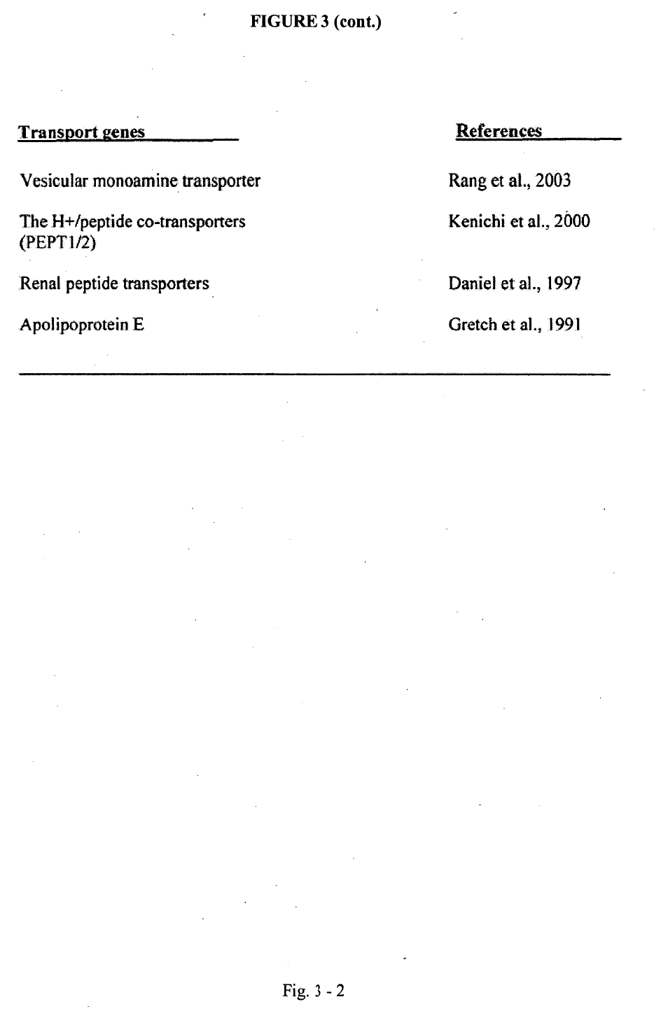

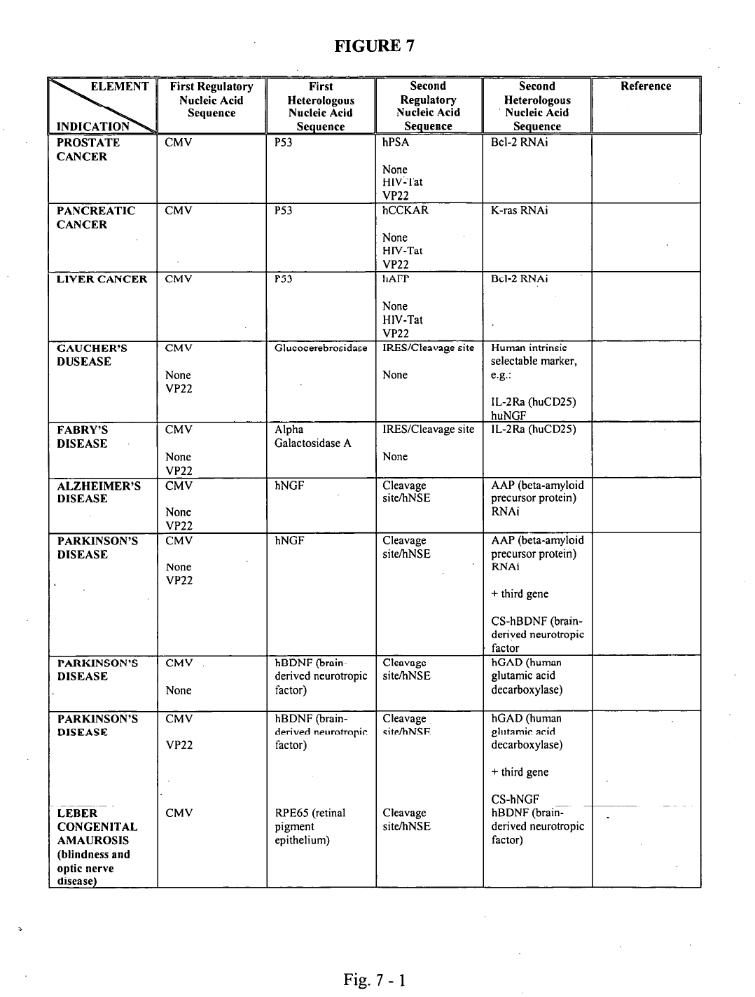

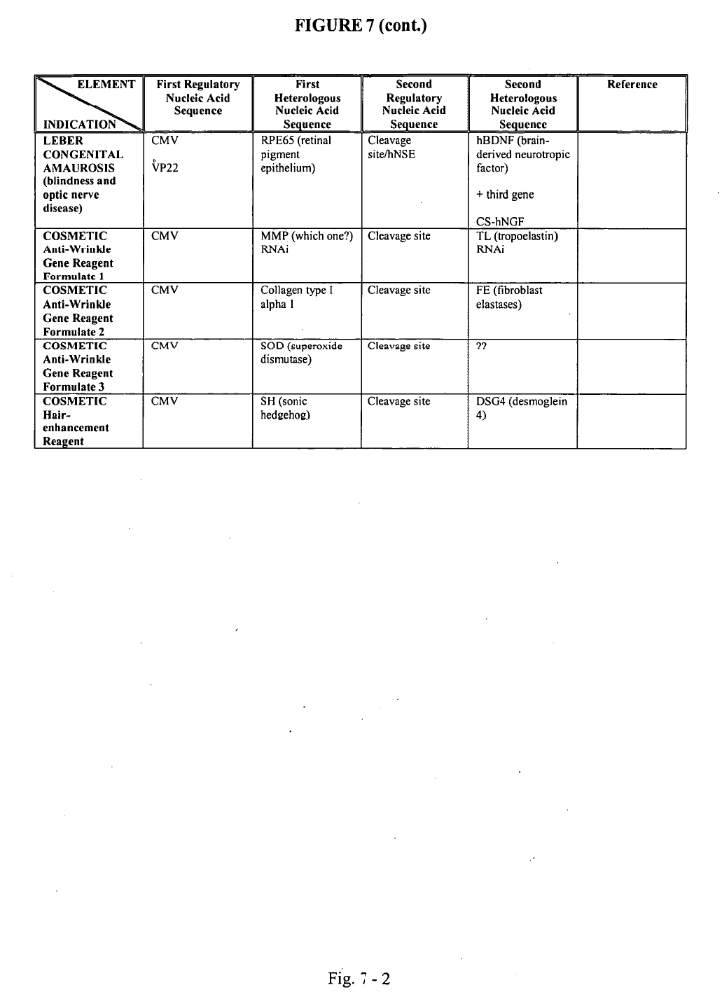

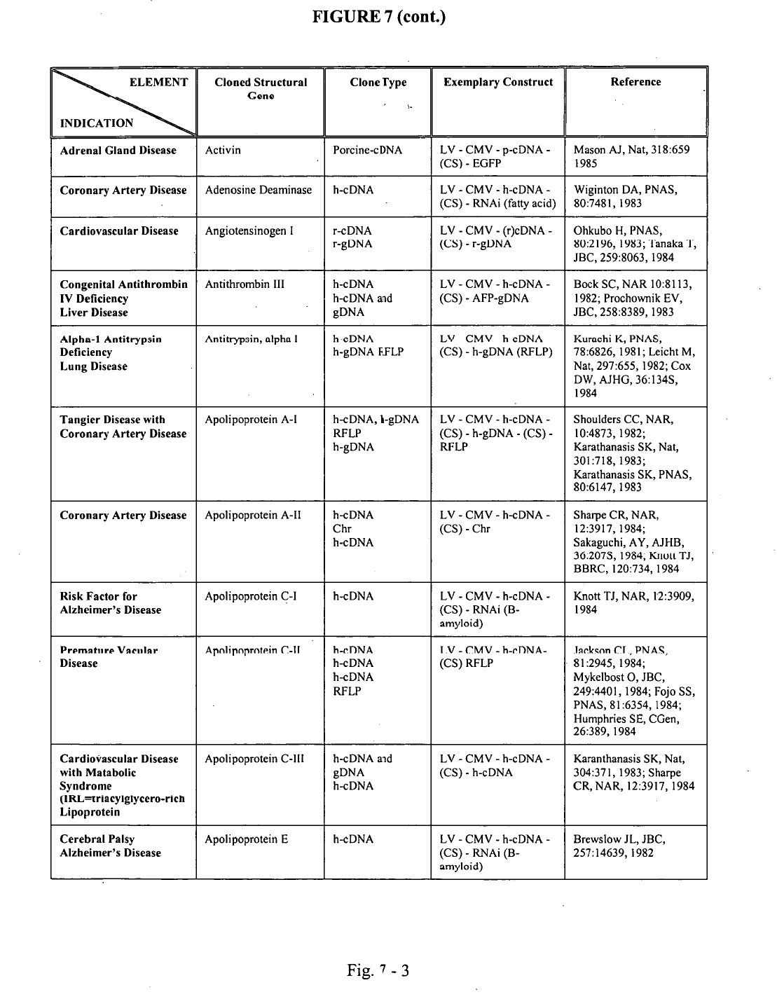

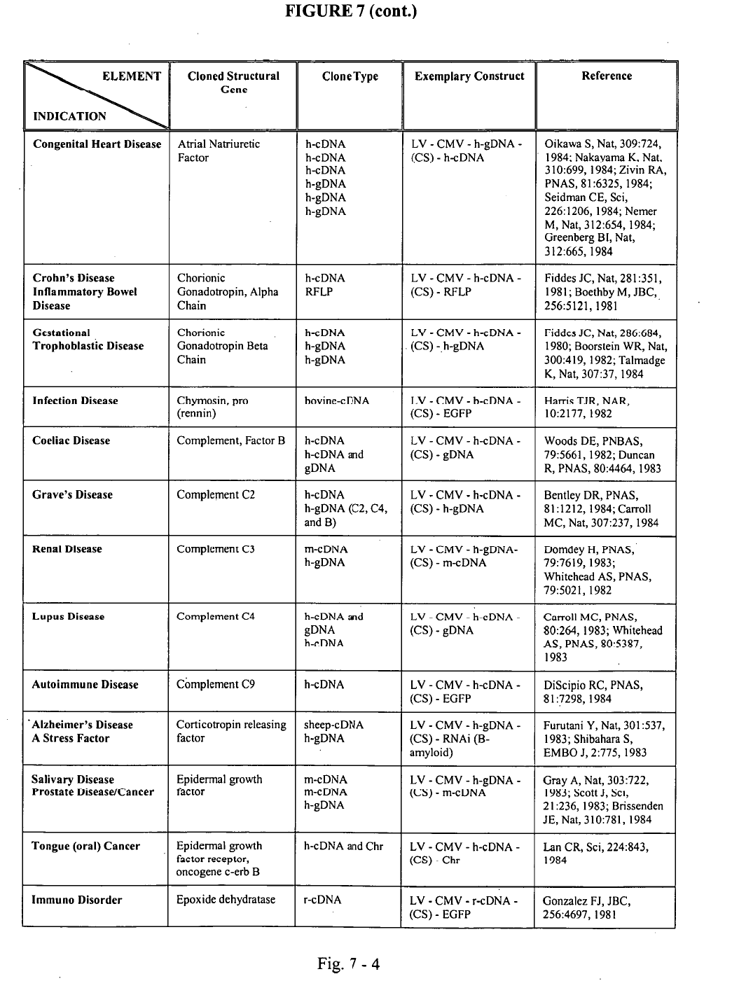

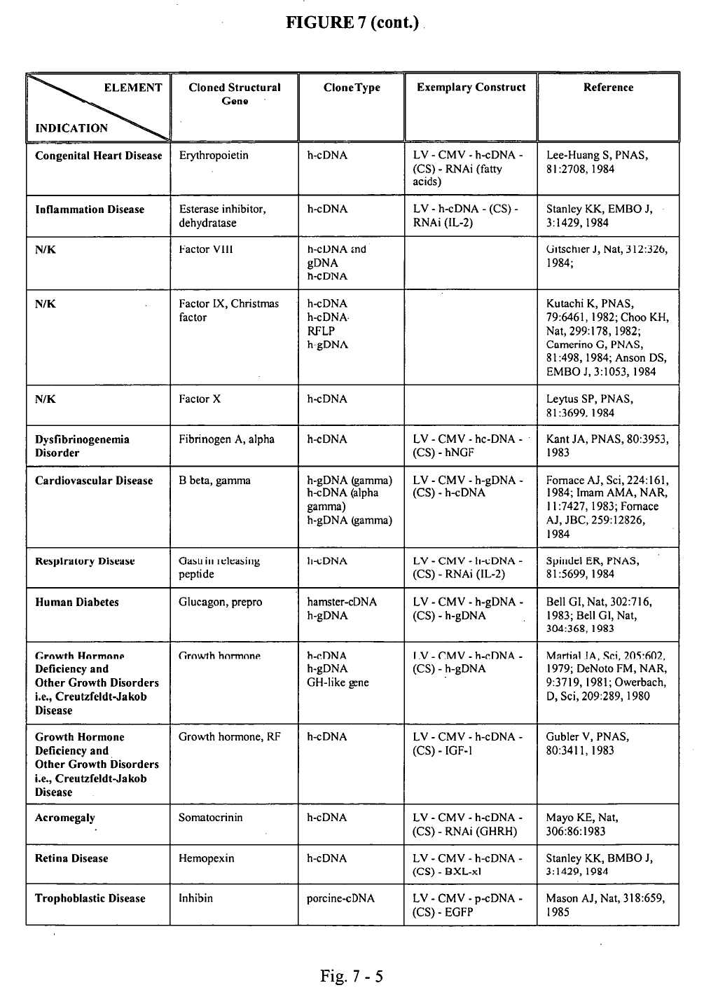

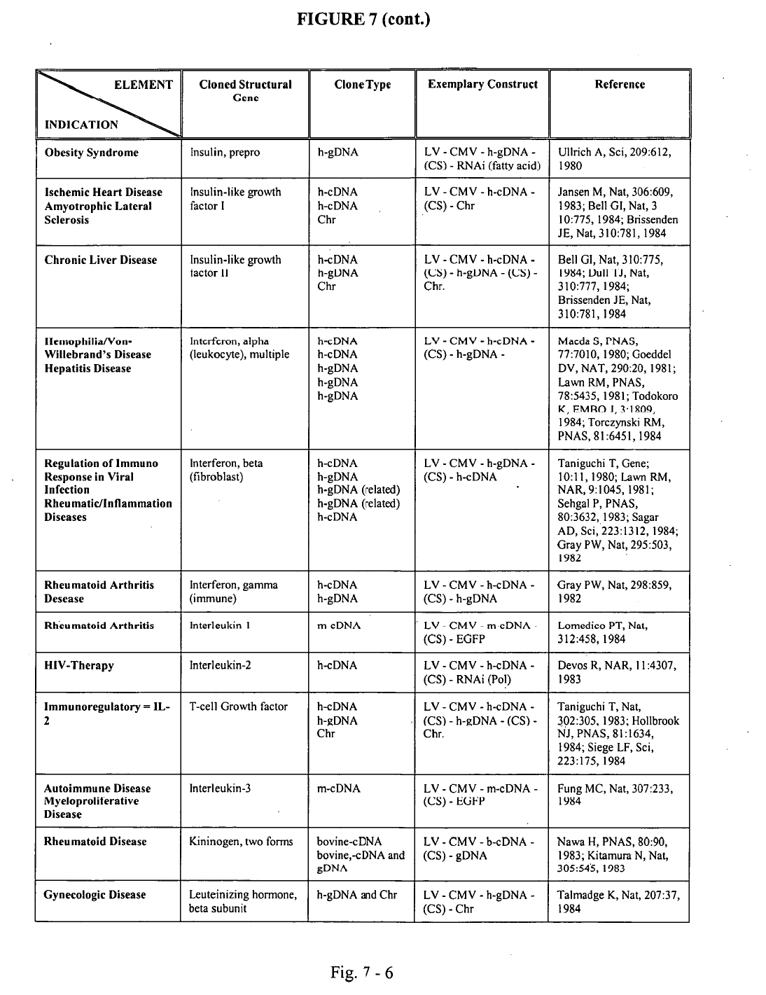

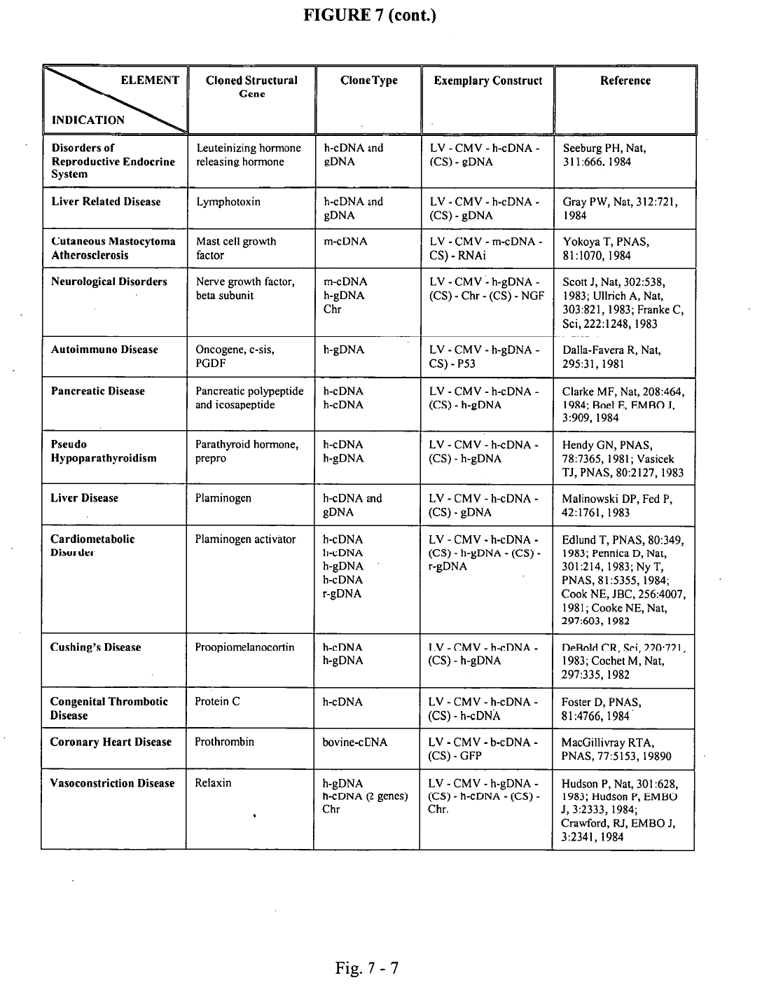

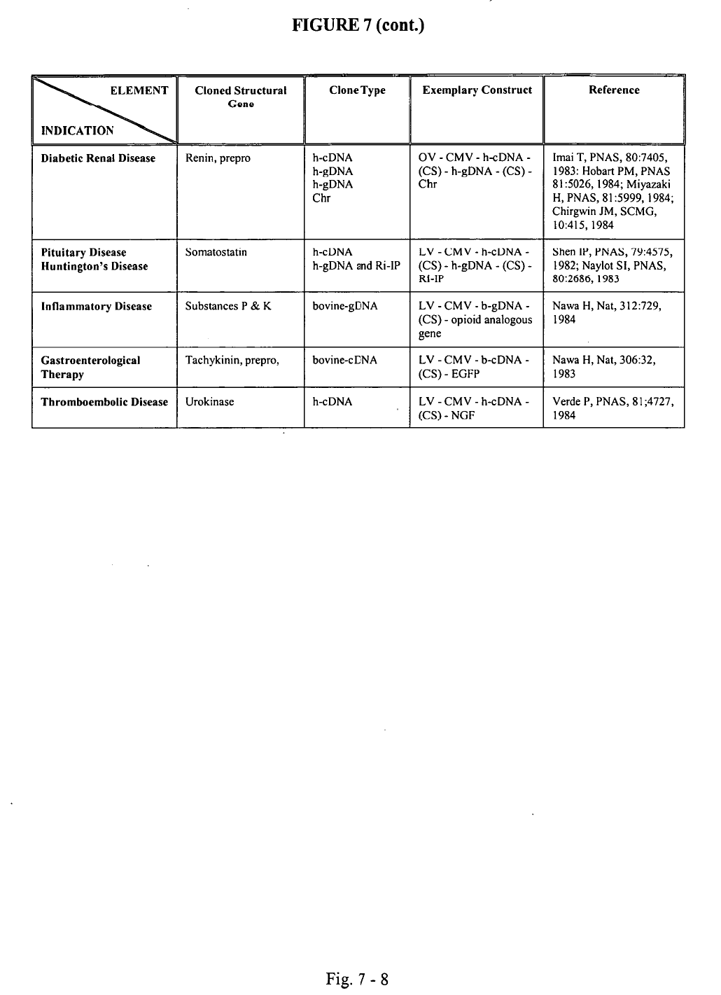

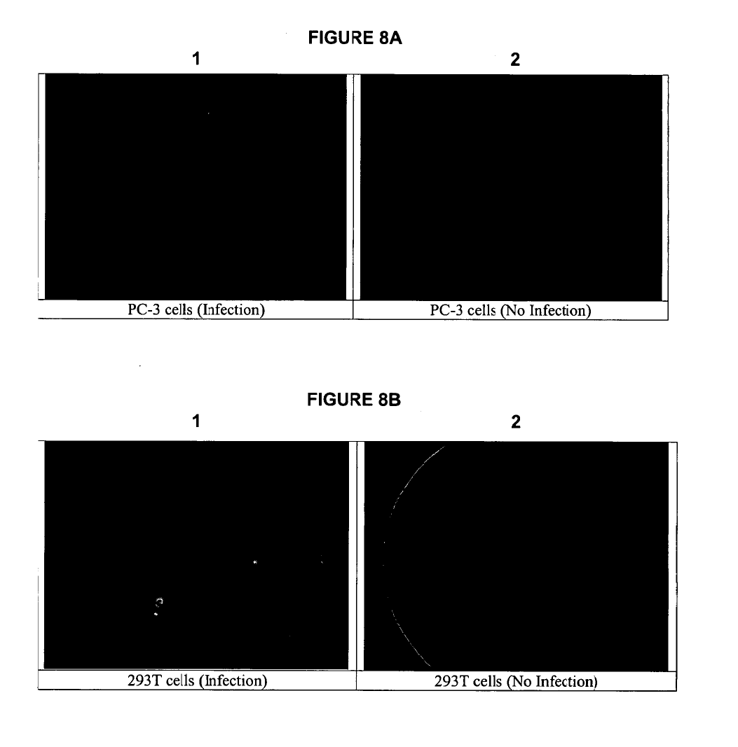

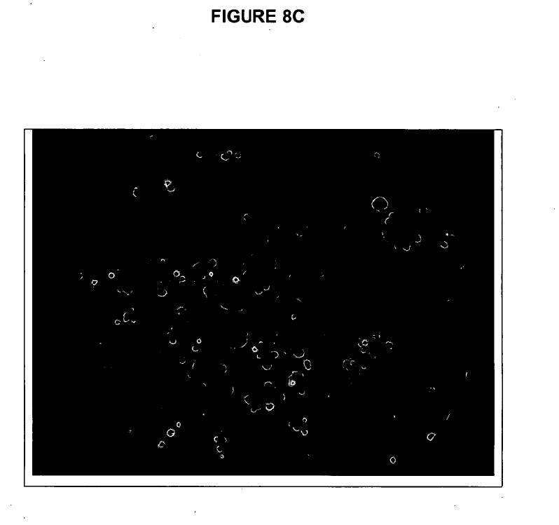

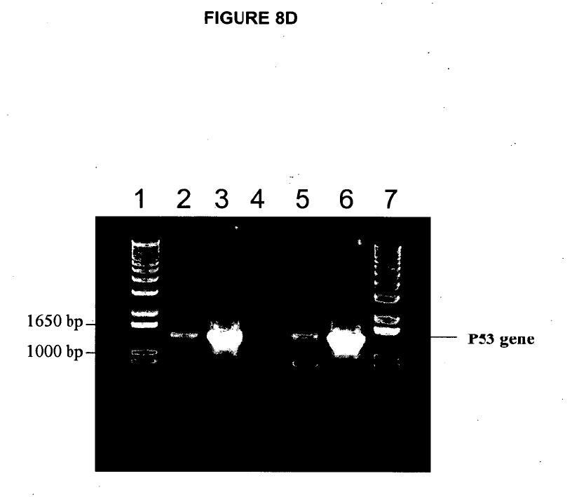

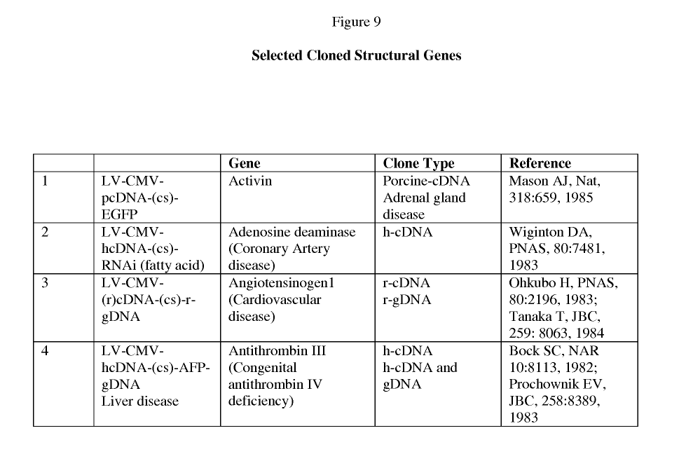

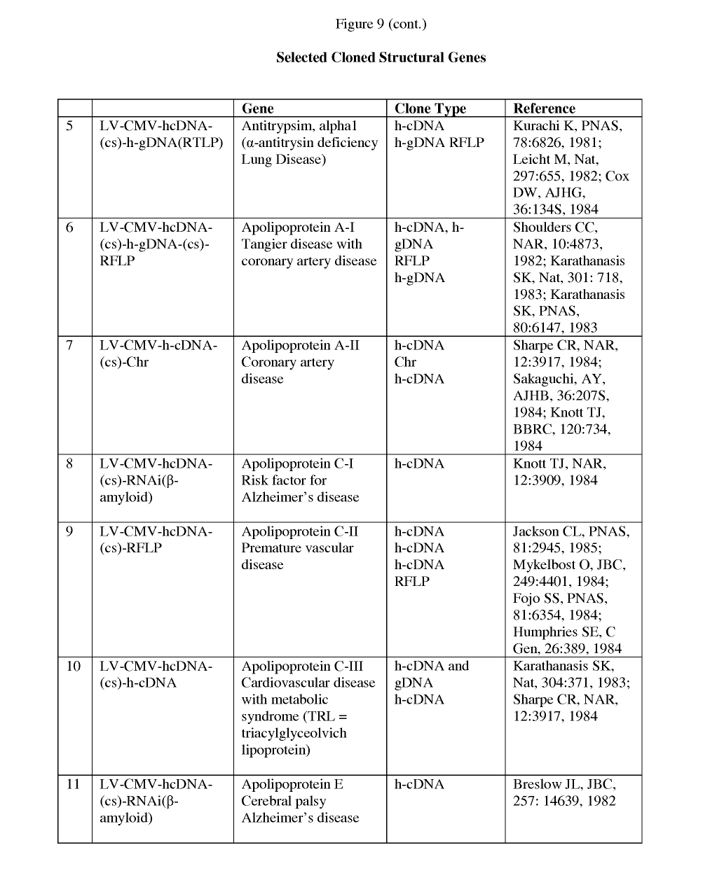

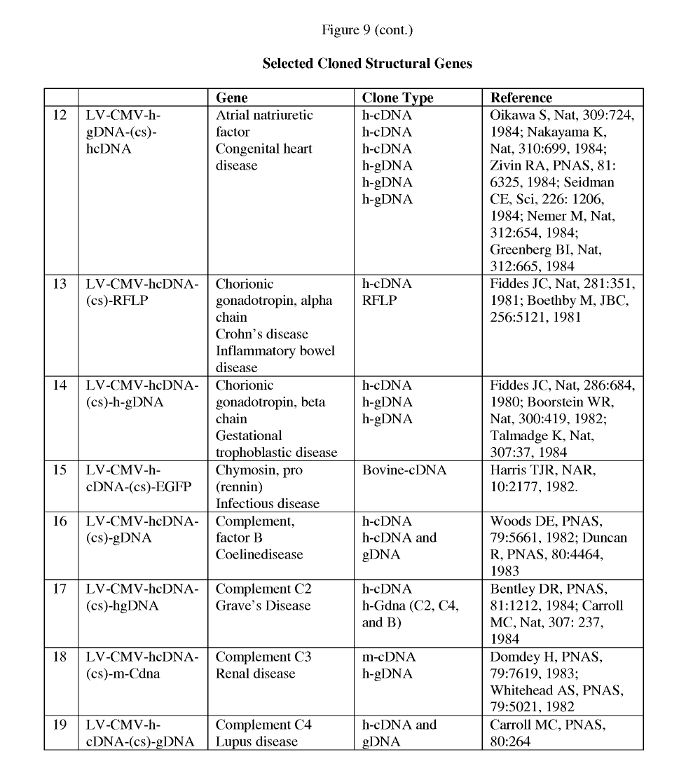

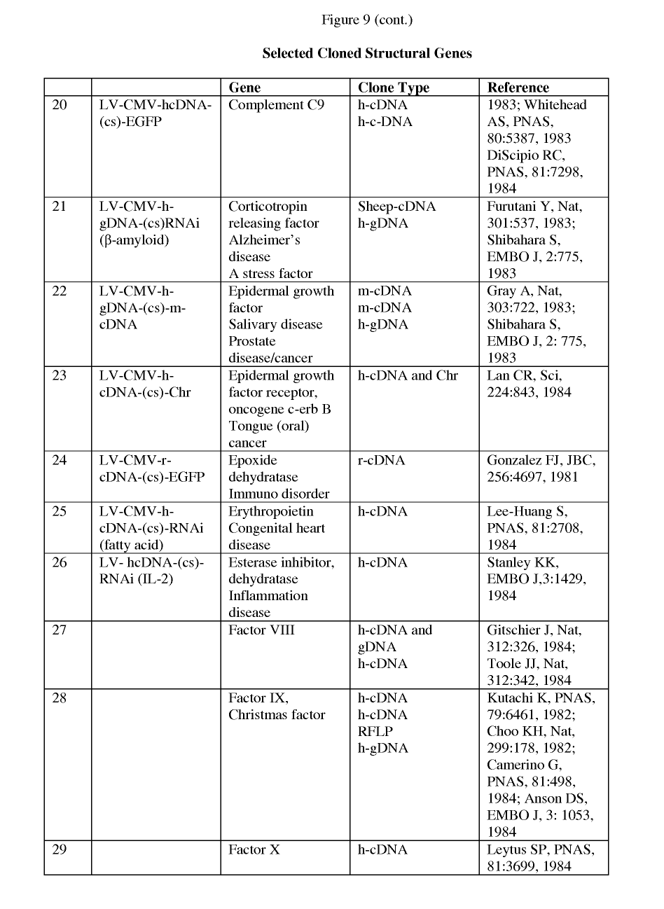

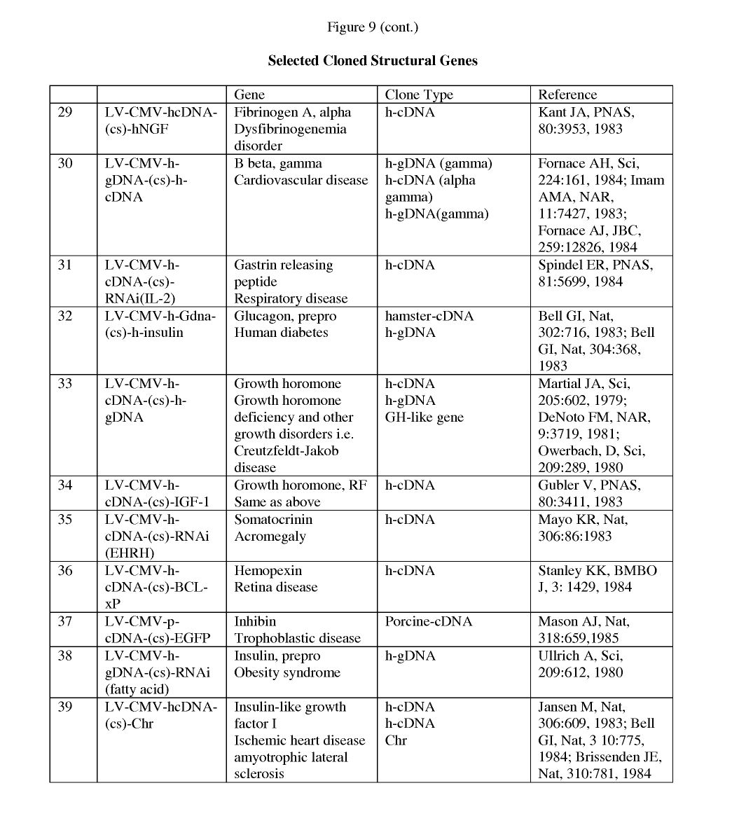

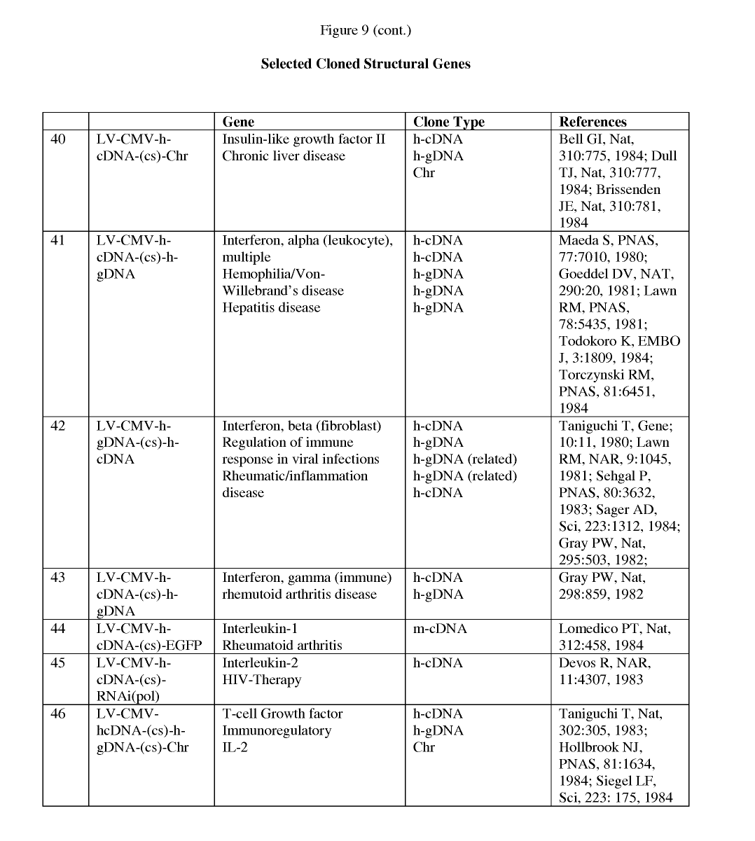

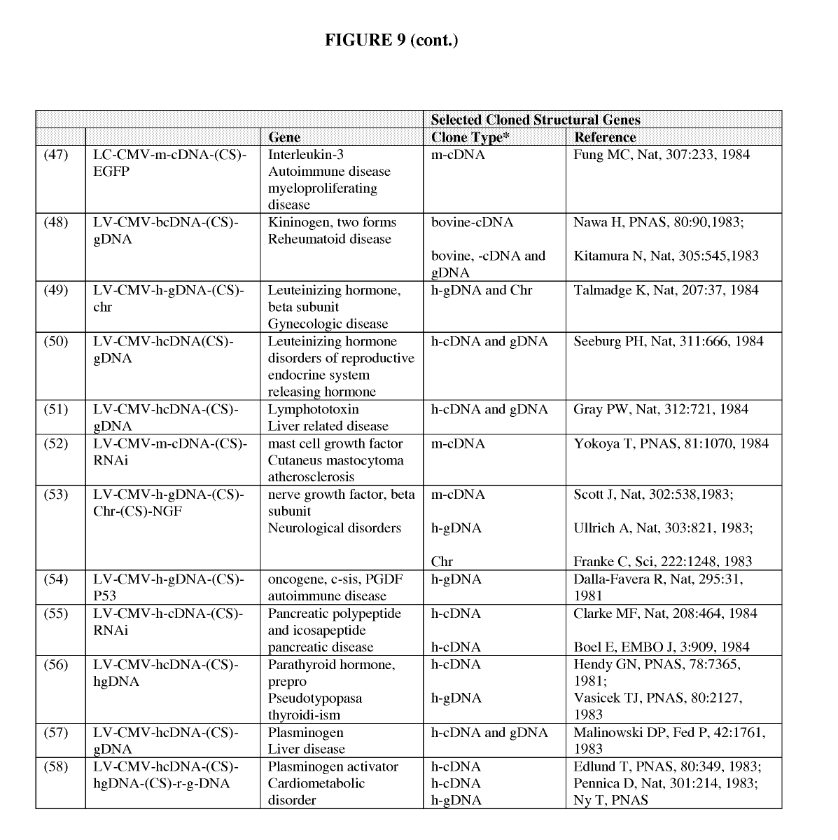

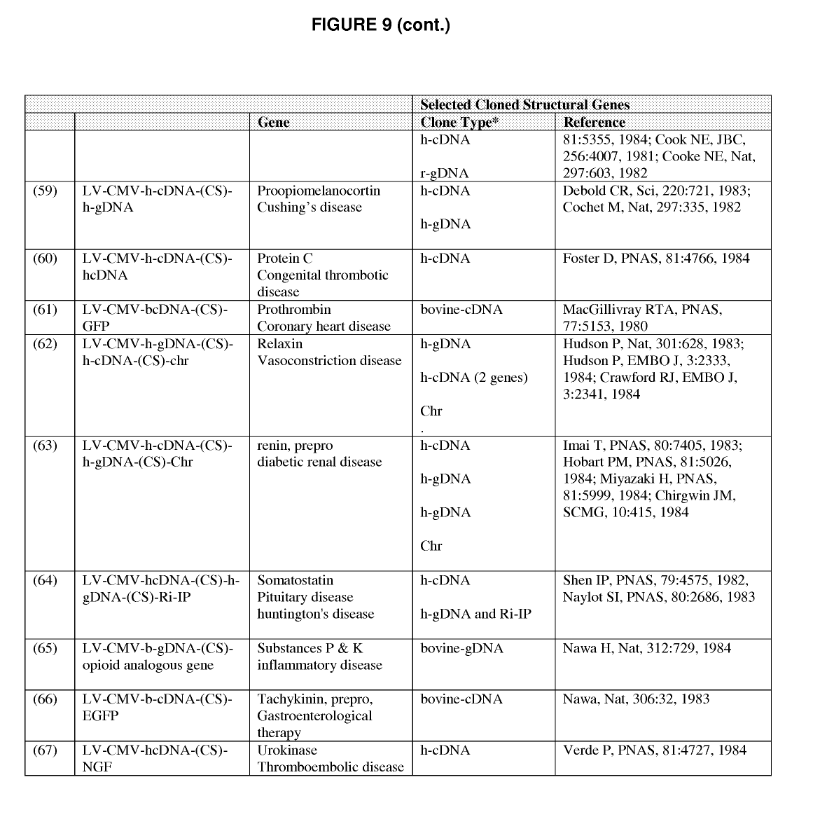

FIG. 2 shows examples of heterologous proteins that can be expressed from their genes using the vectors and methods of the present invention. FIGS. 7 and 8 further list examples of cloned structural genes that can serve as, e.g., a first, second, or third heterologous nucleic acid sequence of the invention.

A preferred protein for expression using the vectors and methods of the present invention is tumor antigen P53. Expression of P53 is defective in most cancers, e.g., due to mutation of the gene or lowered expression. Delivery of the wild-type gene encoding the 53-kilodalton protein is therefore a goal of gene therapy for many cancers.

Nucleic acids encoding the same proteins or targeting the same RNAs can be used in a single transfer vector, for example, two genes for the same protein can be cloned from different sources and used as the first and second heterologous nucleic acid sequences. Similarly, RNAi sequences that are specific for different parts of the same target RNA, or that differ in their percent homology to the target RNA, can be used together.

It may be desirable to transfer a nucleic acid encoding a biological response modifier. Included in this category are immunopotentiating agents including nucleic acids encoding a number of the cytokines classified as “interleukins.” These include, for example, interleukins 1 through 12. Also included in this category, although not necessarily working according to the same mechanisms, are interferons, and in particular gamma interferon (γ-IFN), tumor necrosis factor (TNF) and granulocyte-macrophage-colony stimulating factor (GM-CSF). It may be desirable to deliver such nucleic acids to bone marrow cells or macrophages to treat enzymatic deficiencies or immune defects, or cancer disease. Nucleic acids encoding growth factors, toxic peptides, ligands, receptors, or other physiologically important proteins can also be introduced into specific non-dividing cells.

Selection of RNAi Agents and other Antisense Nucleic Acid Sequences

An RNAi agent used in the vectors and methods of the present invention can be targeted to any RNA molecule. Besides messenger RNA (mRNA), RNAi agents can target, e.g., various species of microRNA. The use of RNAi in gene therapy and RNAi selection and sequence design, are described, e.g., in WO 2007/109131, “Lentiviral Vectors That Provide Improved Expression and Reduced Variegation after Transgenesis,” and WO 2007/087113, “Natural Antisense and Non-Coding RNA Transcripts as Drug Targets,” both of which are incorporated herein by reference.

It may be desirable to modulate the expression of a gene regulating molecule in a cell by the introduction of a molecule by the method of the invention. The term “modulate” envisions the suppression of expression of a gene when it is over-expressed, or augmentation of expression when it is under-expressed. Where a cell proliferative disorder is associated with the expression of a gene, nucleic acid sequences that interfere with the gene's expression at the translational level can be used. This approach utilizes, for example, antisense nucleic acid, ribozymes, or triplex agents, siRNA to block transcription or translation of a specific mRNA, either by masking that mRNA with an antisense nucleic acid or triplex agent, or by cleaving it with a ribozyme.

Antisense nucleic acids are DNA or RNA molecules that are complementary to at least a portion of a specific mRNA molecule (Weintraub, 1990 Scientific American 262:40). In the cell, the antisense nucleic acids hybridize to the corresponding mRNA, forming a double-stranded molecule. The antisense nucleic acids interfere with the translation of the mRNA, since the cell will not translate an mRNA that is double-stranded. Antisense oligomers of about 15 nucleotides are preferred, since they are easily synthesized and are less likely to cause problems than larger molecules when introduced into the target cell. The use of antisense methods to inhibit the in vitro translation of genes is well known in the art (Marcus-Sakura, 1988 Anal Biochem 172:289).

The antisense nucleic acid can be used to block expression of a mutant protein or a dominantly active gene product, such as amyloid precursor protein that accumulates in Alzheimer's disease. Such methods are also useful for the treatment of Huntington's disease, hereditary Parkinsonism, and other diseases. Antisense nucleic acids are also useful for the inhibition of expression of proteins associated with toxicity.

Use of an oligonucleotide to stall transcription is known as the triplex strategy since the oligomer winds around double-helical DNA, forming a three-strand helix. Therefore, these triplex compounds can be designed to recognize a unique site on a chosen gene (Maher, et al. 1991 Antisense Res and Dev 1:227; Helene, C. 1991 Anticancer Drug Design 6:569).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, 1988 J Amer Med Assn 260:3030). A major advantage of this approach is that, because they are sequence-specific, only mRNAs with particular sequences are inactivated.

RNA interference (RNAi) is mediated by double stranded RNA (dsRNA) molecules that have sequence-specific homology to their target nucleic acid sequences (Caplen, N. J., et al, Proc. Natl. Acad. ScL USA 98:9742-9747 (2001)). Biochemical studies in Drosophila cell-free lysates indicate that, in certain embodiments of the present invention, the mediators of RNA-dependent gene silencing are 21-25 nucleotide “small interfering” RNA duplexes (siRNAs). The siRNAs are derived from the processing of dsRNA by an RNase enzyme known as Dicer (Bernstein, E., et al, Nature 409:363-366 (2001)). siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al, Nature 409:363-366 (2001); Boutla, A., et al, Curr. Biol. 11:1776-1780 (2001)). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

“RNAi” or “RNAi agent” refers to an at least partly double-stranded RNA having a structure characteristic of molecules that are known in the art to mediate inhibition of gene expression through an RNAi mechanism or an RNA strand comprising at least partially complementary portions that hybridize to one another to form such a structure. When an RNA comprises complementary regions that hybridize with each other, the RNA will be said to self-hybridize. An RNAi agent includes a portion that is substantially complementary to a target gene. An RNAi agent, optionally includes one or more nucleotide analogs or modifications. One of ordinary skill in the art will recognize that RNAi agents that are synthesized in vitro can include ribonucleotides, deoxyribonucleotides, nucleotide analogs, modified nucleotides or backbones, etc., whereas RNAi agents synthesized intracellularly, e.g., encoded by DNA templates, typically consist of RNA, which may be modified following transcription. Of particular interest herein are short RNAi agents, i.e., RNAi agents consisting of one or more strands that hybridize or self-hybridize to form a structure that comprises a duplex portion between about 15-29 nucleotides in length, optionally having one or more mismatched or unpaired nucleotides within the duplex. RNAi agents include short interfering RNAs (siRNAs), short hairpin RNAs (shRNAs), and other RNA species that can be processed intracellularly to produce shRNAs including, but not limited to, RNA species identical to a naturally occurring miRNA precursor or a designed precursor of an miRNA-like RNA.

The term “short, interfering RNA” (siRNA) refers to a nucleic acid that includes a double-stranded portion between about 15-29 nucleotides in length and optionally further comprises a single-stranded overhang {e.g., 1-6 nucleotides in length) on either or both strands. The double-stranded portion is typically between 17-21 nucleotides in length, e.g., 19 nucleotides in length. The overhangs are typically present on the 3′ end of each strand, are usually 2 nucleotides long, and are composed of DNA or nucleotide analogs. An siRNA may be formed from two RNA strands that hybridize together, or may alternatively be generated from a longer double-stranded RNA or from a single RNA strand that includes a self- hybridizing portion, such as a short hairpin RNA. One of ordinary skill in the art will appreciate that one or more, mismatches or unpaired nucleotides can be present in the duplex formed by the two siRNA strands. One strand of an siRNA (the “antisense” or “guide” strand) includes a portion that hybridizes with a target nucleic acid, e.g., an mRNA transcript. Typically the antisense strand is perfectly complementary to the target over about 15-29 nucleotides, typically between 17-21 nucleotides, e.g., 19 nucleotides, meaning that the siRNA hybridizes to the target transcript without a single mismatch over this length. However, one of ordinary skill in the art will appreciate that one or more mismatches or unpaired nucleotides may be present in a duplex formed between the siRNA strand and the target transcript.

“Short hairpin RNA” refers to a nucleic acid molecule comprising at least two complementary portions hybridized or capable of hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex. The structure may further comprise an overhang. The duplex formed by hybridization of self- complementary portions of the shRNA has similar properties to those of siRNAs and, as described below, shRNAs are processed into siRNAs by the conserved cellular RNAi machinery. Thus shRNAs are precursors of siRNAs and are similarly capable of inhibiting expression of a target transcript. As is the case for siRNA, an shRNA includes a portion that hybridizes with a target nucleic acid, e.g., an mRNA transcript and is usually the perfectly complementary to the target over about 15-29 nucleotides, typically between 17-21 nucleotides, e.g., 19 nucleotides. However, one of ordinary skill in the art will appreciate that one or more mismatches or unpaired nucleotides may be present in a duplex formed between the shRNA strand and the target transcript.

An RNAi agent is considered to be “targeted” to a transcript and to the gene that encodes the transcript if (1) the RNAi agent comprises a portion, e.g., a strand, that is at least approximately 80%, approximately 85%, approximately 90%, approximately 91%, approximately 92%, approximately 93%, approximately 94%, approximately 95%, approximately 96%, approximately 97%, approximately 98%, approximately 99%, or approximately 100% complementary to the transcript over a region about 15-29 nucleotides in length, e.g., a region at least approximately 15, approximately 17, approximately 18, or approximately 19 nucleotides in length; and/or (2) the Tm of a duplex formed by a stretch of 15 nucleotides of one strand of the RNAi agent and a 15 nucleotide portion of the transcript, under conditions (excluding temperature) typically found within the cytoplasm or nucleus of mammalian cells and/or in a Drosophila lysate as described, e.g., in U.S. Patent App. Pubs. 2002/0086356 and 2004/0229266, is no more than approximately 15° C. lower or no more than approximately 10° C. lower, than the Tm of a duplex that would be formed by the same 15 nucleotides of the RNAi agent and its exact complement; and/or (3) the stability of the transcript is reduced in the presence of the RNAi agent as compared with its absence. An RNAi agent targeted to a transcript is also considered targeted to the gene that encodes and directs synthesis of the transcript. A “target region” is a region of a target transcript that hybridizes with an antisense strand of an RNAi agent. A “target transcript” is any RNA that is a target for inhibition by RNA interference. The terms “target RNA” and “target transcript” are used interchangeably herein.

Selection of appropriate RNAi agents is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

Selection of an appropriate antisense nucleic acid is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots can be performed to allow a determination of the degree of identity between genes in target species and other species. By performing Southern blots at varying degrees of stringency, as is well known in the art, it is possible to obtain an approximate measure of identity. These procedures allow the selection of antisense nucleic acids that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

Selection of hybridization sites for antisense nucleic acids can be made by one of skill in the art using methods described in the literature. For example, Ding, et al., report a method for defining mRNA hybridization sites based on determining RNA structures using algorithms and thermodynamic and structural properties of the RNA (Ding, et al., 2001, Statistical prediction of single-stranded regions in RNA secondary structure and application to predicting effective antisense target sites and beyond, Nucleic Acids Research 29(5):1034-1046; incorporated herein by reference in its entirety). Sczakiel, et al., also describe a method for computer-supported design of antisense oligonucleotides (Sczakiel, et al., 2000, Theoretical and experimental approaches to design effective antisense oligonucleotides, Frontiers in Bioscience 5: D194-201; Schen, et al., 2000, RNA accessibility prediction: a theoretical approach is consistent with experimental studies in cell extracts, Nucleic Acids Research 28: 2455-2461; Patzel, et al., 1999, A theoretical approach to select effective antisense oligodeoxyribonucleotides at high statistical probability, J. Biol. Chem. 266:18162-18171; all incorporated herein by reference in their entirety).

Reports of other methods for identifying mRNA hybridization sites used include, e.g., Chiang, et al., who describe a method based on calculating melting temperatures (Chiang, et al., 1991, Antisense oligonucleotides inhibit intercellular adhesion molecule 1 expression by two distinct mechanisms, J. Biol. Chem. 266: 18162-18171, incorporated herein by reference in its entirety). Methods based on calculation of duplex formation free energies have been used (see, e.g., Stull, et al., 1992, Predicting antisense oligonucleotide inhibitory efficacy: a computational approach using histograms and thermodynamic indices, Nucleic Acids Research 20:3501-3508; Ding, et al., 1999, A bayesian statistical algorithm for RNA secondary structure prediction, Comput. Chem. 23:387-400; all incorporated herein by reference in their entirety). Still other methods rely on the use of combinatorial oligonucleotides to identify the hybridization sites within the target RNA. Identification of the hybridization sites is made using RNase H cleavage (Lloyd, et al., 2001, Determination of optimal sites of antisense oligonucleotide cleavage within TNFα mRNA, Nucleic Acids Research 29:3664-3673, incorporated herein by reference in its entirety), microarray analysis (Mir, et al., 1999, Determining the influence of structure on hybridization using oligonucleotide arrays, Nature Biotechnology 17:788-792; and Sohail, et al., 2001, Antisense oligonucleotides selected by hybridization to scanning arrays are effective reagents in vivo, Nucleic Acids Research 29:2041-2051, both incorporated herein by reference in their entirety) or MALDI-TOF mass spectrometry (Altman, et al., 1999, Selection of modified oligonucleotides with increased target affinity via MALDI-monitored nuclease survival assays, J. Comb. Chem. 1:493-508, incorporated herein by reference in its entirety).

The utility of an antisense nucleic acid molecule for modulation (including inhibition) of an mRNA can be readily determined by simple testing. Thus, an in vitro or in vivo expression system comprising the targeted mRNA, mutations or fragments thereof, can be contacted with a particular antisense nucleic acid molecule (modified or un modified) and levels of expression are compared to a control, that is, using the identical expression system which was not contacted with the antisense nucleic acid molecule. In vitro assays of oligonucleotide activity can also be useful for identifying antisense nucleic acids of the invention. For example, Lloyd, et al., report a direct inverse correlation between predicted chimeric antisense oligonucleotide activities, as determined using an in vitro RNase H assay, and the resultant levels of mRNA and protein expression (Lloyd, et al., 2001, Determination of optimal sites of antisense oligonucleotide cleavage within TNFα mRNA, Nucleic Acids Research 29(17): 3664-3673). According to Lloyd, et al., the ability of the in vitro assay to predict oligonucleotide efficacy was superior to other computationally based RNA structural predictions, ΔG calculations and in vivo trial and error methodologies.

Bcl-2 and molecules that work in conjunction with Bcl-2 are also targets for cancer therapy. B cell leukemia/lymphoma-2 (Bcl-2) is the prototype member of a family of cell death regulatory proteins. Bcl-2 is found mainly in the mitochondria and blocks apoptosis by interfering with the activation of caspases. Gene transfer of Bcl-2 into tumor cells has been shown to enhance their metastatic potential (Miyake et al., 1999). Bcl-2 gene transfer may be applied to bone marrow transplant since Bcl-2 enhances the survival of hematopoietic stem cells after reconstitution of irradiated recipient (Innes et al., 1999). Also, Bcl-2 gene transfer could be useful against neurodegenerating diseases since expression of Bcl-2 in neurons protects them from apoptosis (Saille et al., 1999). Bcl-XS (short isoform) is a dominant negative repressor of Bcl-2 and Bcl-XL. It has been used in gene therapy experiments to initiate apoptosis in tumors that express Bcl-2 and Bcl-XL. Expression of Bcl-XS reduces tumor size (Ealovega et al., 1996) and sensitizes tumor cells to chemotherapeutic agents (Sumatran et al., 1995), suggesting a role for Bcl-XS in initiating cell death in tumors that express Bcl-2 or Bcl-XL (Dole et al., 1996). Expression of these genes or RNAi agents targeting them can be selected as appropriate for the condition being treated.

Equivalent Molecules

The invention comprehends that the therapeutic molecules delivered using the vectors and methods of the present invention can be modified. The nucleic acid molecule encoding a given protein be modified, for instance, due to the degeneracy of codon usage, a coding sequence can be modified, and modified and truncated forms of a protein can be used, such as those which may be found in the literature or analogous to truncated or modified forms found in the literature.

Likewise, analogs, homologs, derivatives, and variants of the coding sequences can be used and analogs, homologs, derivatives and variants of proteins can be expressed; such expressed analogs, homologs, derivatives and variants of proteins can have activity analogous to that of the full-length protein, and the analogs, homologs, derivatives and variants of the protein coding sequence encode such active analogs, homologs, derivatives, and variants.

III. Heterologous Regulatory Sequences

A “first heterologous regulatory sequence” is positioned upstream (5′ of) the first heterologous nucleic acid sequence, encoding, e.g., a therapeutic gene. Similarly, a “second heterologous regulatory sequence” can be positioned upstream (5′ of) the second heterologous nucleic acid sequence and downstream of the first heterologous nucleic acid sequence, and a “third heterologous regulatory sequence” can be positioned upstream (5′ of) the third heterologous nucleic acid sequence and downstream of the second heterologous nucleic acid sequence. A heterologous regulatory sequence can be a sequence that influences, e.g., expression or localization, of a therapeutic molecule encoded by a heterologous nucleic acid sequence. A heterologous regulatory sequence can comprise a promoter, enhancer, protease recognition (cleavage) sequence, internal ribosome binding site, intracellular or intercellular trafficking (transport) signal, etc. Certain heterologous regulatory sequences, e.g., cleavage sequences and trafficking sequences, can be expressed as part of a fusion with a therapeutic molecule.

In embodiments, a heterologous regulatory sequence affects an upstream heterologous nucleic acid sequence. Therefore, a fourth regulatory sequence, located downstream of a third heterologous nucleic acid sequence, can be included in the transfer vector. Also, for example, the second heterologous regulatory sequence can contain elements that affect expression or localization of the first heterologous nucleic acid and its corresponding therapeutic molecule, and the third heterologous regulatory sequence can contain elements that affect expression or localization of the second heterologous nucleic acid and its corresponding therapeutic molecule.

Promoters and Enhancers

The promoter sequence may be homologous or heterologous to the desired gene sequence. A wide range of promoters may be utilized, including viral or mammalian promoters. Cell or tissue specific promoters can be utilized to target expression of gene sequences in specific cell populations. Suitable mammalian and viral promoters for the present invention are available in the art.

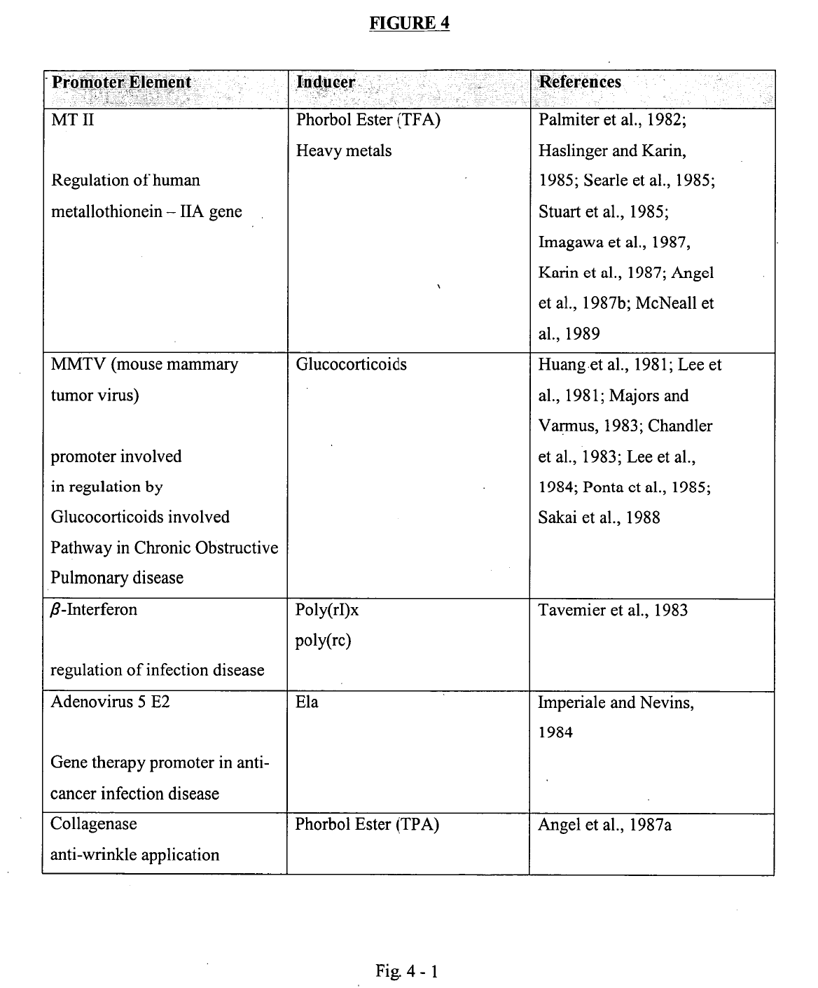

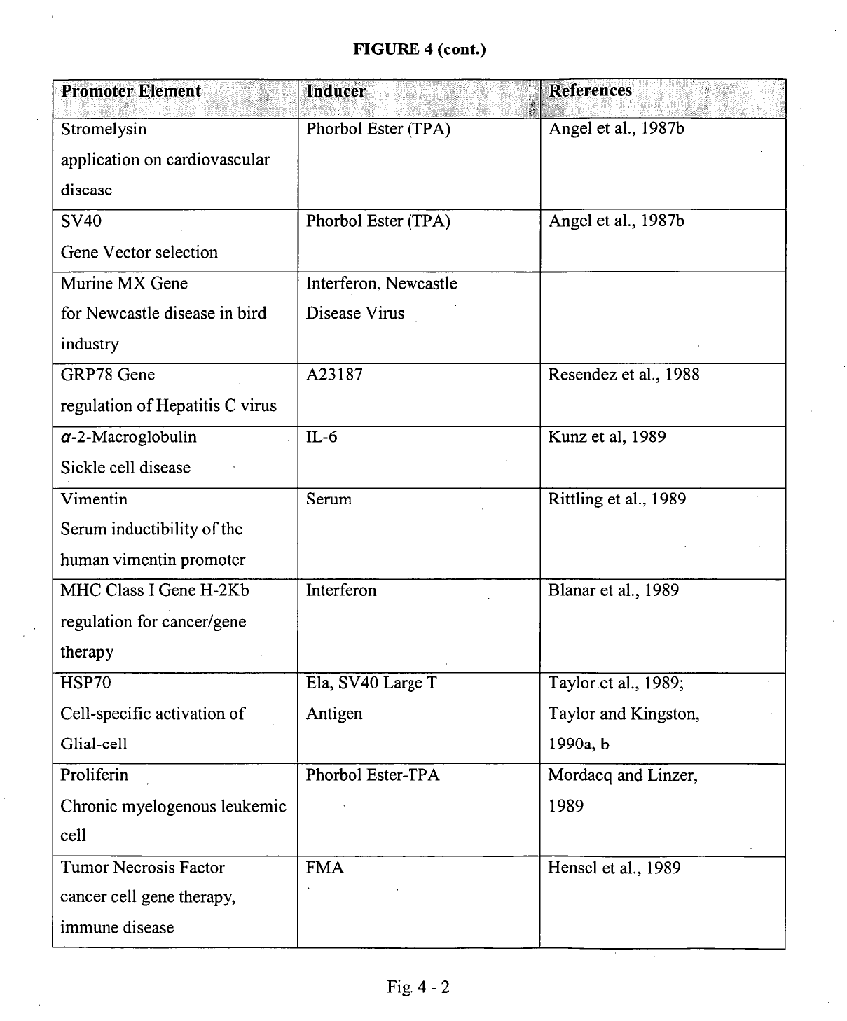

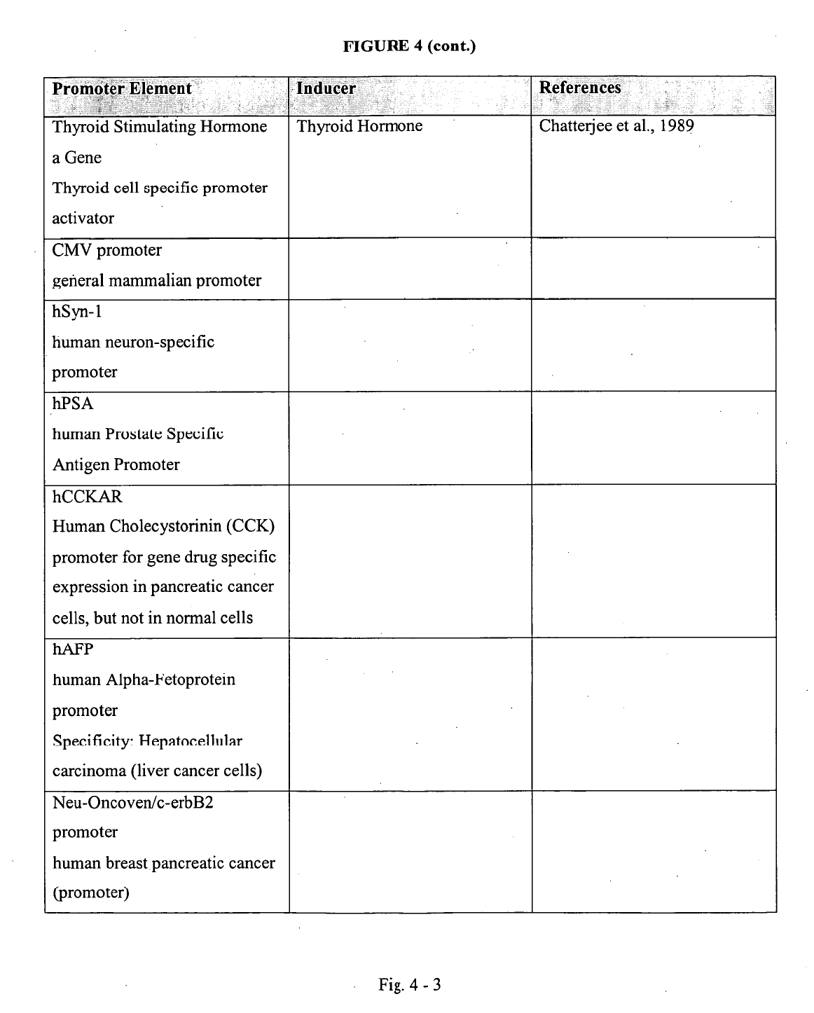

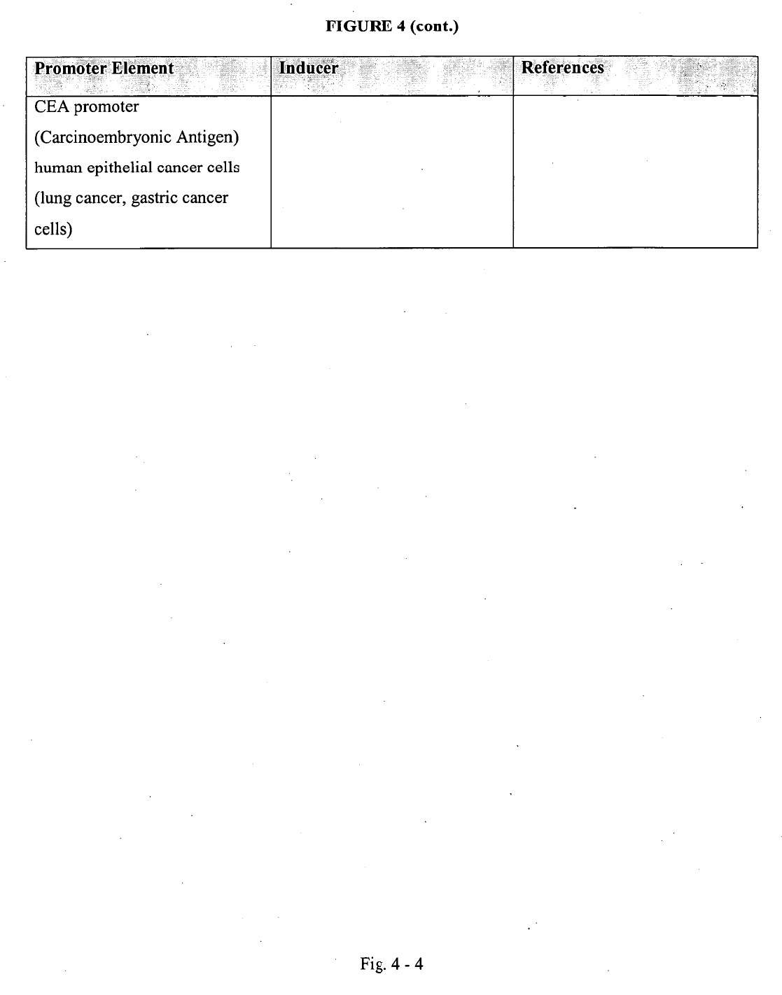

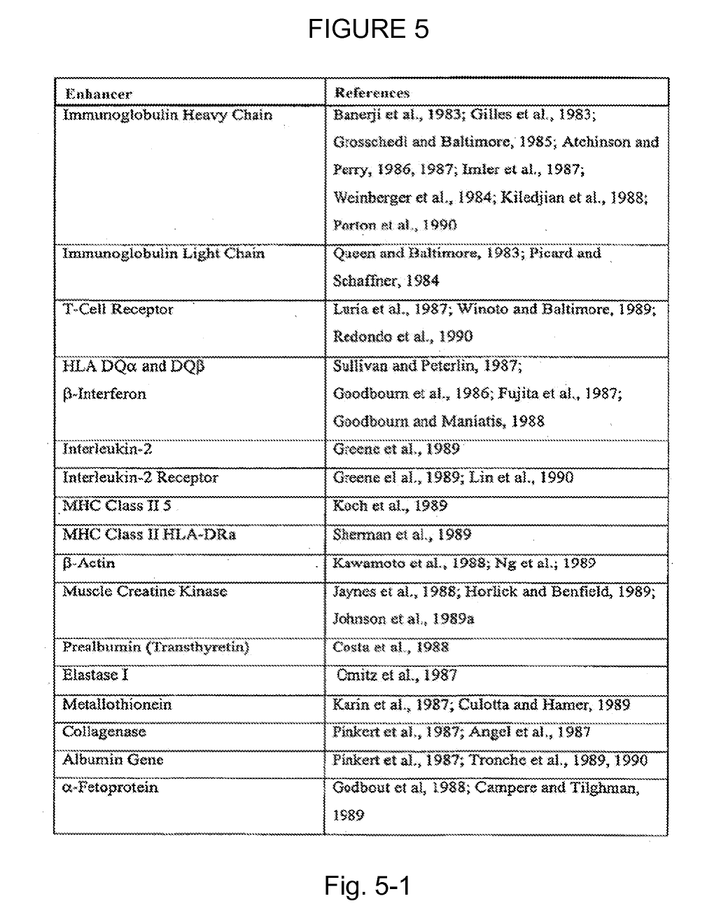

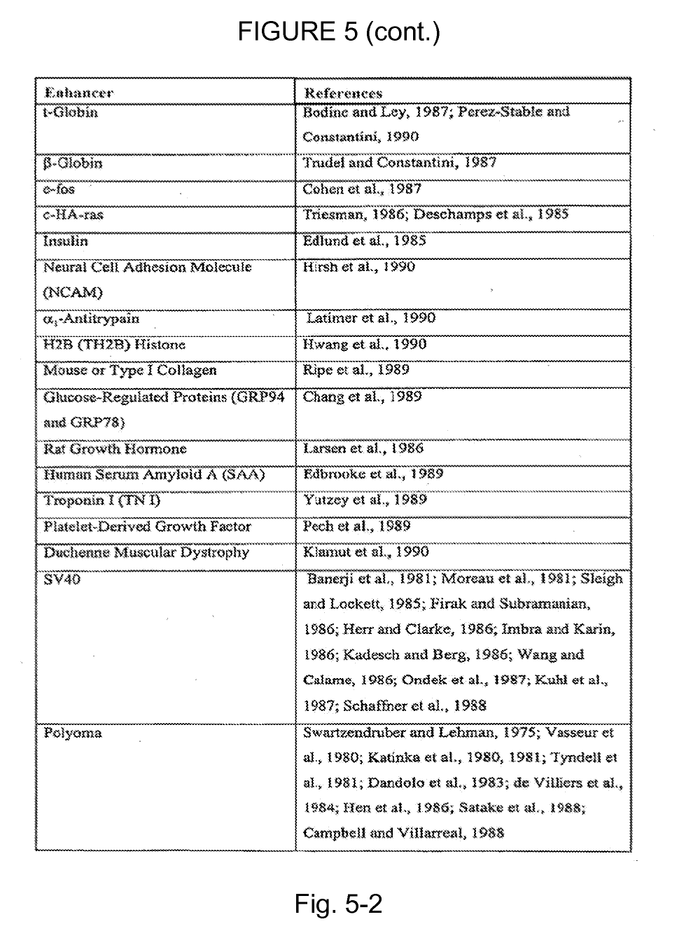

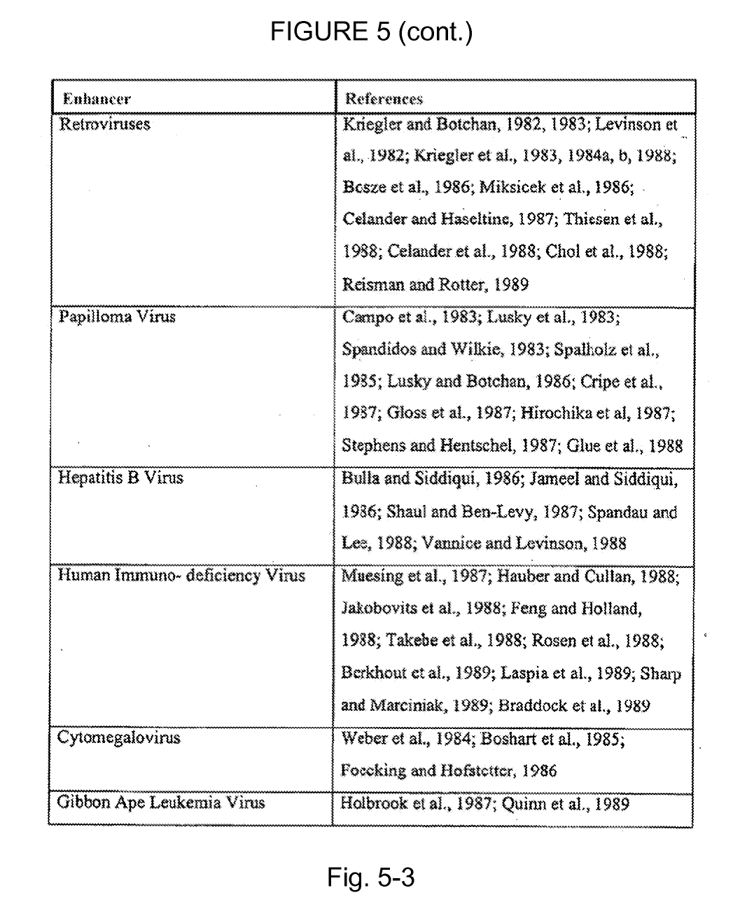

Examples of promoters, cellular promoters/enhancers and inducible promoters/enhancers that can be used in combination with the present invention are listed in FIGS. 4 and 5. Any suitable promoter/enhancer combination (as per the Eukaryotic Promoter Database, EPDB) can be used to drive expression of constructs of the invention.

Trafficking Signals

Trafficking signals can direct a molecule to different compartments within a cell, as well as outside the cell and into other cells. An “intercellular trafficking signal,” or transport signal, is an amino acid sequence that imparts the property to a protein of being able to pass through membranes between cells. Examples of membrane-penetrating proteins include, but are not limited to, several plant and bacterial protein toxins, such as ricin, abrin, modeccin, diphtheria toxin, cholera toxin, anthrax toxin, heat labile toxins, and Pseudomonas aeruginosa exotoxin A. Examples of membrane-penetrating proteins that are not toxins include the TAT protein of human immunodeficiency virus and the protein VP22, the product of the UL49 gene of herpes simplex virus type 1. One line of research involves adapting such molecules from their naturally destructive role into therapeutic compositions.

The effectiveness of lentiviral vectors to deliver genes encoding proteins fused to herpes simplex virus type 1 tegument protein VP22 has been reported (see, e.g., U.S. Pat. App. Pub. No. 2003/0119770). “VP22” denotes: protein VP22 of HSV, e.g., of HSV1, and transport-active fragments and homologues thereof, including transport-active homologues from other herpesviruses including varicella zoster virus VZV, marek's disease virus MDV and bovine herpesvirus BHV.

Among sub-sequences of herpesviral VP22 protein with transport activity, investigators have found that, for example, transport activity is present in polypeptides corresponding to amino acids 60-301 and 159-301 of the full HSV1 VP22 sequence (1-301). A polypeptide consisting of aa 175-301 of the VP22 sequence has markedly less transport activity, and is less preferred in connection with the present invention. Accordingly, the present invention relates in one aspect to a sub-sequence of VP22 containing a sequence starting preferably from about aa 159 (or earlier, towards the N-terminal, in the native VP22 sequence), to about aa 301, and having (relative to the full VP22 sequence) at least one deletion of at least part of the VP22 sequence which can extend for example from the N-terminal to the cited starting point, e.g., a deletion of all or part of the sequence of about aa 1-158. (Less preferably, such a deletion can extend further in the C-terminal direction, e.g., to about aa 175.) For example, partial sequences in the range from about aa 60-301 to about aa 159-301 are provided.

VP22 sequences as contemplated herein extend to homologous proteins and fragments based on sequences of VP22 protein homologues from other herpesviruses, e.g., the invention provides corresponding derivatives and uses of the known VP22-homologue sequences from VZV (e.g., all or homologous parts of the sequence from aa 1-302), from MDV (e.g., all or homologous parts of the sequence from aa 1-249) and from BHV (e.g., all or homologous parts of the sequence from aa 1-258). The sequences of the corresponding proteins from HSV2, VZV, BHV and MDV are available in public protein/nucleic acid sequence databases. Thus, for example, within the EMBL/Genbank database, a VP22 sequence from HSV2 is available as gene item UL49 under accession no. Z86099 containing the complete genome of HSV2 strain HG52; the complete genome of VZV including the homologous gene/protein is available under accession numbers X04370, M14891, M16612; the corresponding protein sequence from BHV is available as “bovine herpesvirus 1 virion tegument protein” under accession number U21137; and the corresponding sequence from MDV is available as gene item UL49 under accession number L10283 for “gallid herpesvirus type 1 homologous sequence genes”. In these proteins, especially those from HSV2 and VZV, corresponding deletions can be made, e.g., of sequences homologous to aa 1-159 of VP22 from HSV1. Homologies between these sequences are readily accessible by the use of standard algorithms, default parameters, and software.

Furthermore, chimeric VP22 proteins and protein sequences are also useful within the context of the present invention, e.g., a protein sequence from VP22 of HSV1 for part of which a homologous sequence from the corresponding VP22 homologue of another herpesvirus has been substituted. For example, into the sequence of polypeptide 159-301 from VP22 of HSV1, C-terminal sequences can be substituted from VP22 of HSV2 or from the VP22 homologue of BHV.

Investigators have found that deletion of the 34-amino acid C-terminal sequence from VP22 of HSV1 abolishes transport-activity, thus this sequence region contains essential elements for transport activity. According to a further aspect of the invention, there are provided in-frame fusions comprising a nucleic acid sequence encoding the 34-amino acid C-terminal sequence from VP22, or a variant thereof, together with a sequence for a heterologous nucleic acid sequence. In-frame fusions of nucleic acid sequences encoding modified terminal fragments having at least one mutation insertion or deletion relative to the C-terminal 34 amino acid sequence of HSV1 VP22 are also provided.

Investigators have also been found that sequences necessary for transport activity contain one or a plurality of amino acid sequence motifs or their homologues from the C-terminal sequence of VP22 of HSV1 or other herpesviruses, which can be selected from RSASR (SEQ ID NO: 1), RTASR (SEQ ID NO: 2), RSRAR (SEQ ID NO: 3), RTRAR (SEQ ID NO: 4), ATATR (SEQ ID NO 5), and wherein the third or fourth residue A can be duplicated, e.g., as in RSAASR (SEQ ID NO: 6). Corresponding in-frame fusions of nucleic acid sequences encoding these signals are also provided.

The HIV-1 Tat protein was also reported to enhance intercellular trafficking in vitro. It is composed of 86 amino acids and contains a highly basic region and a cysteine-rich region. It was found that Tat-derived peptides as short as eleven amino acids are sufficient for transduction of proteins (Fawell, et al., 1994, Proc Natl Acad Sci USA 91:664-668). However, the exact mechanism by which the 11-amino acid transduction domain crosses lipid bilayers is poorly understood. Schwarze et al. (Science, 385:1569-1572, 1999) reported generating a Tat-β-galactosidase fusion protein that was delivered efficiently into brain tissue and skeletal muscle in vivo.

In embodiments of the present invention, a Tat-derived trafficking protein of eleven amino acids Try-Gly-Arg-Lys-lys-Arg-Arg-Gln-Arg-Arg (SEQ ID NO: 7) is used to enhance intercellular trafficking of therapeutic molecules. Any appropriate nucleic acid sequence can be used to express this protein, e.g., tat ggc agg aag aag cgg aga cag cga cga aga (SEQ ID NO:8) with a start codon.

Control of intracellular as well as intercellular transport is contemplated for use in the methods of the invention. This level of control can be used to target therapeutic proteins to particular cellular compartments, e.g., to correct defects for proteins involved in specific disease processes.

Other potentially useful Tat-derived trafficking sequences have been described. For example Chauhan, et al., 2007, The Taming of the Cell Penetrating Domain of the HIV Tat: Myths and Realities, J. Control Release 117(2): 148-162, incorporated herein by reference in its entirety, disclose variants of the Tat protein transduction domain.

Following are Tat-derived cell penetrating peptides described by Chauhan, et al.:

| | | |
|---|---|---|
| PTD | YGRKKRRQRRR | (SEQ ID NO: 9) |
| PTD-4 | YARAAARQARA | (SEQ ID NO: 10) |
| YM-3 | THRLPRRRRRR | (SEQ ID NO: 11) |
| CTP | GGRRARRRRRR | (SEQ ID NO: 12) |

As reported by the authors, depending on the nature of the protein being transported, these peptides effect transport, including transport among cellular compartments. For example, cytoplasmic proteins reportedly end up in the nucleus when PTD is used, nucleo-cytoplasmic proteins go to the nucleus when PTD-4 is used, secretory proteins are found in the nucleus and outside the cell when YM-3 is used, and membrane proteins go to the membrane and nucleus when CTP is used.

Other cell penetrating proteins useful for introducing recombinant proteins into cells are penetratin, polylysine, polyarginine, Kaposi FGF, Syn Bl, FGF-4, nuclear localization signal, anthrax toxin derivative 254-amino acids peptide segment, diphtheria toxin “R” binding domain, MPG (described below), WR peptide, and exotoxin A. Penetratin peptide has also been used for siRNA delivery to cells. In embodiments, these proteins or their derivatives used in as trafficking signals in the vectors and methods of the invention.

A fusion peptide, “MPG,” has been described for efficient transduction of nucleic acids. This peptide is a bipartite amphipathic peptide obtained by combining the fusion domain of HIV-gp41 protein and the NLS domain of SV40 large T antigen. This peptide is being used as a nanoparticle for transduction of siRNA in vitro and is also available commercially. (See, e.g., Chauhan, et al., 2007; Morris, et al., 1999, A novel potent strategy for gene delivery using a single peptide vector as a carrier, Nucleic Acids Research 27:3510-3517.)

In embodiments of the vectors and methods of the invention, an siRNA or other antisense agent is transported intercellularly or intracellularly.

The inventive transfer vector may include splice acceptor and splice donor sequences flanking the gene construct. In the case of multigene transfer vector, the splice acceptor sequence may be inserted upstream of the first promoter-structural gene unit and splice donor site may be located downstream of the last gene unit in the 5′ to 3′ order. A mammalian insulator sequence (MIS) may be inserted downstream of the gene units, or if specific promoters are used for the gene units, then the MIS may be placed before the first gene unit and after the last gene unit followed by splice donor site. A translation initiation sequence may also be included in the multigene gene transfer vector. The use of the translation initiation sequence causes the second and subsequent multigene units to be expressed evenly and stably compared with the first gene expression product.

Translation initiation sequence

The transfer vector may optionally comprise a sequence that allows for translation initiation in the middle of a messenger RNA (mRNA) sequence as part of the greater process of protein synthesis. While this sequence may be typically IRES other sequences may be used, which may have a similar sequence or function.

Splice acceptor/Splice donor sequence

The transfer vector may optionally comprise a gene unit flanked by a splice acceptor site 5′ to a gene unit and a splice donor site 3′ to the promoter-gene. For instance, the following elements may be present 5′ to 3′: a splice acceptor site, first promoter for the first gene unit, first heterologous nucleic acid sequence and splice donor site 3′ to the first gene unit, then a splice acceptor site and a second promoter for the second gene, the second heterologous nucleic acid sequence, splice donor site, and then if there is a third gene, a splice acceptor site, third promoter for the third gene unit, the third heterologous nucleic acid sequence, and a splice donor site, and so forth. The splice acceptor or donor site typically may include about 5 to 10 bases.

Mammalian insulator sequence (MIS)

The transfer vector may optionally comprise an insulator sequence, in particular mammalian insulator sequence (MIS). Insulators are DNA sequence elements that can protect against the activation influence of distal enhancers associated with other genes, and also help to preserve the independent function of genes embedded in a genome in which they are surrounded by regulatory signals so that cross interaction is avoided. The insulators as used in the present application may not necessarily be limited to use in lentiviruses. The insulators may be used with other viral vectors.

To provide background on these insulator sequences, the zinc finger protein CCCTC-binding factor (CTCF) is a versatile transcription regulator that binds to insulators and shows enhancer-blocking activity for regulating gene expression control. Chicken (beta-globin) insulator with about 1.2 kb is widely used in vitro or in vivo animals, but generally does not have human or mammalian compatible factors to be entirely useful in treating humans or mammals.

Bovine or human growth hormone transcriptional stop sequences may also be used as insulators. A short-element of about 238 bp containing the “HS” DNA element core sequence, which is the binding site for CTCF is also effective as an “enhancer blocking” element. Template DNA for generation of such element, for example a pcDNA3 , which contains bovine growth hormone transcriptional stop sequence may be as follows: PCR primers: 5′-agctagatagtgtcacctaaatgc-3′ (SEQ ID NO:13) and 5′-agcatgcctgctatt-3′ (SEQ ID NO:14).

A binding site for the transcription factor CTCF may be responsible for enhancer-blocking activity in a variety of insulators, including the insulators at the 5′ and 3′ chromatin boundaries of the chicken and human beta-globin locus. The minimal element responsible for this activity may be a binding site for CTCF.

When several different sequences of insulators of 5′ and 3′ HS/CTCF are compared using “human insulator” as the template, there are at least two mutations in the mouse, at least 5 mutations in chicken at the 5′ HS/CTCF. In addition, there are at least two mutations in mouse, at least 4 mutations at the 3′ HS/CTCF.

The insulator sequence as used in the lentivirus transfer vector may be placed as follows. In a single gene vector where a general promoter is used to control gene expression, MIS may be placed 3′ of the gene and upstream of the splicing donor site. However, if a specific promoter is used to control the expression of the gene, the MIS may be placed upstream of the specific promoter and downstream of the gene, wherein the MIS sequences are optionally flanked by the splice acceptor on the 5′ side and splice donor on the 3′ side of the gene. In a single gene construct, for RNAi expression where specific promoter is used, MIS may flank the specific promoter-structural gene construct, optionally with a splice donor site downstream of the 3′ MIS. Two or more MIS may be used together to enhance the blocking effects.

For multiple gene vectors, if a general promoter is used, an MIS may be included downstream of the gene units with splice donor site 3′ to the MIS. However, if a specific promoter is used MIS is place upstream of the first specific promoter for the first structural gene, and another MIS downstream of the last specific promoter-structural gene set. The MIS are optionally flanked by splice acceptor on the 5′ side and splice donor on the 3′ side.

IV. Therapeutic Applications

The invention includes a variety of therapeutic applications for the lentiviral vectors of the invention. In particular, lentiviral vectors are useful for gene therapy. Exemplary therapeutic applications are listed in FIG. 7. The invention provides methods of treating and/or preventing infection by an infectious agent, the method comprising administering to a subject prior to, simultaneously with, or after exposure of the subject to the infectious agent a composition comprising an effective amount of a lentiviral vector, wherein the lentiviral vector directs transcription of at least one RNA that hybridizes to form an shRNA or siRNA that is targeted to a transcript produced during infection by the infectious agent, which transcript is characterized in that reduction in levels of the transcript delays, prevents, and/or inhibits one or more aspects of infection by and/or replication of the infectious agent.

The invention provides methods of treating a disease or clinical condition by, for example, removing a population of cells from a subject at risk of or suffering from the disease or clinical condition and engineering or manipulating the cells to comprise an effective amount of therapeutic agents by infecting or transfecting the cells with a lentiviral vector. At least a portion of the cells are returned to the subject.

Without limitation, therapeutic approaches may find particular use in diseases such as cancer, in which a mutation in a cellular gene is responsible for or contributes to the pathogenesis of the disease, and in which specific inhibition of the target transcript bearing the mutation may be achieved by expressing an RNAi agent targeted to the target transcript within the cells, without interfering with expression of the normal (i.e. non-mutated) allele. Furthermore, treatment of any cancer in which P53 expression is defective is contemplated using the vectors and methods of the invention.

The invention is also useful for the treatment of genetic diseases, for example, Gaucher's Disease and Fabry's Disease. Gaucher's disease is a lysosomal storage disease caused by a deficiency of the enzyme glucocerebrosidase. This deficiency leads to an accumulation of the enzyme substrate, the fatty substance glucocerebroside (also known as glucosylceramide). Fatty material can collect in the spleen, liver, kidneys, lungs, brain and bone marrow. It has been reported, using a mouse model for Gaucher's Disease, that a lentiviral vector can transduce HSCs that are capable of long-term gene expression in vivo (Kim, et al., 2005, “Long-term expression of the human glucocerebrosidase gene in vivo after transplantation of bone-marrow-derived cells transformed with a lentivirus vector, J. Gene Med. 7:878-887, incorporated herein by reference).

According to certain embodiments of the invention, rather than removing cells from the body of a subject, infecting or transfecting them in tissue culture, and then returning them to the subject, inventive lentiviral vectors or lentiviruses are delivered directly to the subject.

Neurological Disorders

Cells infected with a recombinant lentivirus of the invention, in vivo, or ex vivo, used for treatment of a neuronal disorder for example, may optionally contain an exogenous gene, for example, a gene which encodes a receptor or a gene which encodes a ligand. Such receptors include receptors which respond to dopamine, GABA, adrenaline, noradrenaline, serotonin, glutamate, acetylcholine and other neuropeptides, as described above. Examples of ligands which may provide a therapeutic effect in a neuronal disorder include dopamine, adrenaline, noradrenaline, acetylcholine, gamma-aminobutyric acid and serotonin. The diffusion and uptake of a required ligand after secretion by an infected donor cell would be beneficial in a disorder where the subject's neural cell is defective in the production of such a gene product. A cell genetically modified to secrete a neurotrophic factor, such as nerve growth factor (NGF), might be used to prevent degeneration of cholinergic neurons that might otherwise die without treatment. Alternatively, cells can be grafted into a subject with a disorder of the basal ganglia, such as Parkinson's disease, or can be modified to contain an exogenous gene encoding L-DOPA, the precursor to dopamine. Parkinson's disease is characterized by a loss of dopamine neurons in the substantia nigra of the midbrain, which have the basal ganglia as their major target organ.

U.S. Pat. No. 6,800,281, “Lentiviral-mediated growth factor gene therapy for neurodegenerative diseases,” incorporated herein by reference in its entirety, describes methods for treating Parkinson's Disease using glial cell derived neurotrophic factor (GDNF), highly conserved neurotrophic factor that potently promotes the survival of many types of neurons.

Parkinson's disease (PD) is a neurodegenerative disorder characterized by the loss of the nigrostriatal pathway; a progressive disorder resulting from degeneration of dopaminergic neurons within the substantia nigra. Although the cause of Parkinson's disease is not known, it is associated with the progressive death of dopaminergic (tyrosine hydroxylase (TH) positive) mesencephalic neurons, inducing motor impairment. The characteristic symptoms of Parkinson's disease appear when up to 70% of TH-positive nigrostriatal neurons have degenerated. Surgical therapies aimed at replacing lost dopaminergic neurons or disrupting aberrant basal ganglia circuitry have recently been tested (C. Honey et al. 1999). However, these clinical trials have focused on patients with advanced disease, and the primary goal of forestalling disease progression in newly diagnosed patients has yet to be realized. The administration can be by stereotaxic injection. The administration can be intracranially, e.g., intracranially to stiatum or to substantia nigra. The administration can also be by retrograde transport.

In an embodiment, the administration site is the striatum of the brain, in particular the caudate putamen. Injection into the putamen can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra.

In another embodiment the vector system is injected directly into the spinal cord. This administration site accesses distal connections in the brain stem and cortex. Within a given target site, the vector system may transduce a target cell. The target cell may be a cell found in nervous tissue, such as a neuron, astrocyte, oligodendrocyte, microglia or ependymal cell. In a preferred embodiment, the target cell is a neuron, in particular a TH positive neuron.

The vector system can be administered by direct injection. Methods for injection into the brain (in particular the striatum) are well known in the art (Bilang-Bleuel et al (1997) Proc. Acad. Nati. Sci. USA 94:8818-8823; Choi-Lundberg et al (1998) Exp. Neurol.154:261-275; Choi-Lundberg et al (1997) Science 275:838-841; and Mandel et al (1997)) Proc. Acad. Natl. Sci. USA 94:14083-14088). Stereotaxic injections may be given.

Administration of the cells or virus into selected regions of the recipient subject's brain may be made by drilling a hole and piercing the dura to permit the needle of a microsyringe to be inserted. The cells or recombinant lentivirus can alternatively be injected intrathecally into the spinal cord region. A cell preparation infected ex vivo, or the recombinant lentivirus of the invention, permits grafting of neuronal cells to any predetermined site in the brain or spinal cord, and allows multiple grafting simultaneously in several different sites using the same cell suspension or viral suspension and permits mixtures of cells from different anatomical regions.

For transduction in tissues such as the brain, it is necessary to use very small volumes, so the viral preparation is concentrated by ultracentrifugation. The resulting preparation should have at least 10⁸ pfu./ml, preferably from 10⁸ to 10¹⁰ pfu./ml, more preferably at least 10⁹ pfu./ml. (The titer is expressed in transducing units per ml (pfu./ml) as titred on a standard D17 cell line). It has been found that improved dispersion of transgene expression can be obtained by increasing the number of injection sites and decreasing the rate of injection (Horellou and Mallet (1997) as above). Usually between 1 and 10 injection sites are used, more commonly between 2 and 6. For a dose comprising 1-5×10⁹ pfu./ml, the rate of injection is commonly between 0.1 and 10 μl/min, usually about 1 μl/min.

In another embodiment the vector system is administered to a peripheral administration site. The vector may be administered to any part of the body from which it can travel to the target site by retrograde transport. In other words the vector may be administered to any part of the body to which a neuron within the target site projects.

The “periphery” can be considered to be all part of the body other than the CNS (brain and spinal cord). In particular, peripheral sites are those which are distant to the CNS. Sensory neurons may be accessed by administration to any tissue which is innervated by the neuron. In particular this includes the skin, muscles and the sciatic nerve.

In another embodiment the vector system is administered intramuscularly. In this way, the system can access a distant target site via the neurons which innervate the innoculated muscle. The vector system may thus be used to access the CNS (in particular the spinal cord), obviating the need for direct injection into this tissue. There is thus provided a non-invasive method for transducing a neuron within the CNS. Muscular administration also enables multiple doses to be administered over a prolonged period.

Other neuronal disorders that can be treated similarly by the method of the invention include Alzheimer's disease, Huntington's disease, neuronal damage due to stroke, and damage in the spinal cord. Alzheimer's disease is characterized by degeneration of the cholinergic neurons of the basal forebrain. The neurotransmitter for these neurons is acetylcholine, which is necessary for their survival. Engraftment of cholinergic cells infected with a recombinant lentivirus of the invention containing an exogenous gene for a factor which would promote survival of these neurons can be accomplished by the method of the invention, as described. Following a stroke, there is selective loss of cells in the CA1 of the hippocampus as well as cortical cell loss which may underlie cognitive function and memory loss in these patients. Once identified, molecules responsible for CA1 cell death can be inhibited by the methods of this invention. For example, antisense sequences, or a gene encoding an antagonist can be transferred to a neuronal cell and implanted into the hippocampal region of the brain.

The method of transferring nucleic acid also contemplates the grafting of neuroblasts in combination with other therapeutic procedures useful in the treatment of disorders of the CNS. For example, the lentiviral infected cells can be co-administered with agents such as growth factors, gangliosides, antibiotics, neurotransmitters, neurohormones, toxins, neurite promoting molecules and antimetabolites and precursors of these molecules such as the precursor of dopamine, L-DOPA.

Further, there are a number of inherited neurologic diseases in which defective genes may be replaced including: lysosomal storage diseases such as those involving β-hexosamimidase or glucocerebrosidase; deficiencies in hypoxanthine phosphoribosyl transferase activity (the “Lesch-Nyhan” syndrome”); amyloid polyneuropathies (-prealbumin); Duchenne's muscular dystrophy, and retinoblastoma, for example.

For diseases due to deficiency of a protein product, gene transfer could introduce a normal gene into the affected tissues for replacement therapy, as well as to create animal models for the disease using antisense mutations. For example, it may be desirable to insert a Factor IX encoding nucleic acid into a lentivirus for infection of a muscle or liver cell.

Stem cell therapy contemplates injection of stem cells transduced by a lentiviral vector carrying a therapeutic gene of interest into a fetus central nervous system. The correction or rescue of a genetic defect is achieved during cell differentiation. Stem cells at a nondividing stage should be efficiently transduced by such a vector using a convenient infection technique.

V. Pharmaceutical Compositions

The invention further provides pharmaceutical compositions comprising lentiviral vectors of the invention and one or more pharmaceutically acceptable carriers.

The pharmacologically active compounds of this invention can be processed in accordance with conventional methods of galenic pharmacy to produce medicinal agents for administration to patients, e.g., mammals including humans.

The compounds of this invention can be employed in admixture with conventional excipients, i.e., pharmaceutically acceptable organic or inorganic carrier substances suitable for parenteral, enteral (e.g., oral) or topical application, which do not deleteriously react with the active compounds. Suitable pharmaceutically acceptable carriers include but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, particularly suitable are injectable, sterile solutions, preferably oily or aqueous solutions, as well as suspensions, emulsions, or implants, including suppositories. Ampoules are convenient unit dosages.

For enteral application, particularly suitable are tablets, dragees, liquids, drops, suppositories, or capsules. A syrup, elixir, or the like can be used wherein a sweetened vehicle is employed.

Sustained or directed release compositions can be formulated, e.g., by inclusion in liposomes or those wherein the active compound is protected with differentially degradable coatings, e.g., by microencapsulation, multiple coatings, etc. It is also possible to freeze-dry these compounds and use the lyophilizates obtained, for example, for the preparation of products for injection.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

It will be appreciated that the actual preferred amounts of active compound in a specific case will vary according to the specific compound being utilized, the compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The lentiviral vectors of the invention can also be administered in combination with other agents, for example, chemotherapeutic agents, radiation treatment, or steroids, according to methods known and described in the art. PCT Publication WO 2008/08069942, “Novel Methods of Enhancing Delivery of a Gene Therapy Vector Using Steroids,” describes methods for enhancing expression of a viral vector-encoded therapeutic gene product by delivering to a subject the viral vector in conjunction with a steroid, e.g., prednisolone, cortisone, corticosterone, or dexamethasone. In embodiments, the individual therapies being combined are not necessarily administered together, e.g., they can be administered separately via different modes of administration, alternately, etc.

VII. Patients

The invention contemplates treatment of patients including human patients. The term patient as used in the present application refers to all different types of mammals including humans and the present invention is effective with respect to all such mammals. The present invention is effective in treating any mammalian species which have a disease potentially remedied by delivery of a gene product or inhibition of expression of a gene.

Liver Cancer Treatment

In addition to the regulation of P53 and Bcl-2, other genes may also be regulated. Regulation of cell cycle progression is dysfunctional in HCC. The oncogene, C-MYC, has been implicated in the pathogenesis of HBV induced HCC. The MYC gene is one of the most often dysregulated genes in human cancer, especially HCC. Overexpression and amplification of the C-MYC gene is an early event in HCC development.

The relentless DNA synthesis cycling cells requires a steady supply of nucleosides. Targeting the biochemical synthesis pathways that feed this pathway has proven to be effective pharmacologically. This pathway may be targeted by inhibiting M2 subunit of ribonucleotide reductase (RRM2), which has been shown to be a potent inhibitor of tumor cell growth.

The caveat to targeting C-MYC and RRM2 is that both are involved in the normal functions of many types of cells. Therefore, systemic down regulation of their protein level may cause adverse effect similar to those observed with conventional cytotoxic chemotherapy and radiotherapy. However, the potential for a dramatic therapeutic effect with C-MYC and RRM2 justifies their inclusion in testing.

Finally, tumor vascularization is vital to survival of cancer cells, so cancers almost universally co-opt signaling pathways that drive vascularization. HCC is highly vesicular and critically depend on angiogenesis. A key component of these pathways is vascular endothelial growth factor (VEGF). In HCC, tumor cells, endothelial cells, and pericytes secrete a net excess level of VEGF. This pathway may be intervened by molecularly downregulating VEGF.

Dose-response on tumors

The effect of the therapeutic lentivirus vectors on tumor size are tested in immunodeficient mice bearing HepG2 tumor xenografts. Briefly, 50 nude mice(Balb/c Nu/Nu nude mice; 4-6 weeks old; approximately 20 g; Jackson Lab) are inoculated with 2×10⁶ HepG2 cells in 0.2 mL of sterile Matrigel by subcutaneous injection on their dorsal aspect. Tumors are allowed to grow to approximately 200 mm³ (approximately 7 days post-inoculation) before commencing administration of virus or control solutions.

The experimental groups are listed in Table 1. Two different routes of administration are tested: intratumoral injection (IT) and intravenous injection (IV). The two control groups are i) the untreated group and ii) the empty vector (lentiviral vector without therapeutic gene). The four treatment groups are treated with a) JZ2, b) GXY-TA-AFP-shRNAmir, c) GXY-TA-AFP-p53-shRNAmir, or d) a mixture of JZ2 and GXY-TA-AFP-shRNAmir.

For IT administration, 1 dose of virus (10⁸ TU/mL to 10⁹ TU/mL) in 0.1 mL of saline are administered using a 25 ga needle. The dosing is repeated on days 5, 9, 14, and 16. For IV administration, 1 dose of viral protein in 0.05 mL is administered by tail vein injection. The dosing is repeated on days 5, 9, 14, and 16.

TABLE 1
Treatment Groups

| Treatment | Injection methods | note |
|---|---|---|
| Vehicle (saline) | IT | Control group |
| Empty vector | IT | Control group |
| CMV-p53 vector | IT | Treatment group |
| TA-AFP-four siRNAs | IT | Treatment group |
| vector | | |
| CMV-p53 vector and TA- | IT | Treatment group |
| AFP-four siRNAs vector | | |
| TA-AFP-p53+four | IT | Treatment group |
| siRNAs vector | | |
| Vehicle (saline) | IV | Control group |
| Empty vector | IV | Control group |
| CMV-p53 vector | IV | Treatment group |
| TA-AFP-four siRNAs | IV | Treatment group |
| vector | | |
| CMV-p53 vector and TA- | IV | Treatment group |
| AFP-foursiRNAs vector | | |
| TA-AFP-p53+four | IV | Treatment group |
| siRNAs vector | | |

IT: intratumoral; IV: intravenous

Efficacy Observation: Tumor Volume Measurement

Tumors are measured in their longest dimension and perpendicular to their longest dimension on days 0, 5, 9, 14, and 21. Tumor volume is calculated using the formula (longest dimension) x (width)²/2. Results are expressed as volume and relative change in volume over time.

Upon sacrifice of the tumor bearing mice at the end of the experiment, samples of tissue from tumor, skin, muscle, liver, brain, heart, and kidney are harvested from selected mice and flash frozen until analysis for expression of viral and therapeutic genes.

Relationship of Change in Body Weight to Treatment

The weight of each mouse is measured immediately prior to treatment on Day 0. Subsequent measurements on Days 0, 5, 9, 14, and 21 are performed immediately prior to treatment or sacrifice (for last measurement).

Upon sacrifice of the mice at the end of the experiment, tumors (or residual connective tissue, if present) are dissected, weighed, and frozen. In addition, samples of tissue from tumor, skin, muscle, liver, brain, heart, and kidney are harvested from all mice and flash frozen until analysis for expression of viral and therapeutic genes.

Biodistribution of Viral Vector DNA

The distribution of vector are determined by using a qPCR based method to measure the number of proviral copies per unit of tissue mass cell by normalizing it to the number of endogenous gene amplicons. The detailed method is very similar to that of titration methods described above. The tissue genomic DNA are isolated and served as template for provirus amplicon (WPRE) and endogenous gene amplicon (GAPDH). In addition to the JZ2 vector standard curve to determine copy number of the proviral DNA, a GAPDH plasmid standard curve is used to determine the copy number of endogenous gene GAPDH. The biodistribution rate in one specific tissue is calculated by normalizing copy numbers of proviral DNA to that of GAPDH amplicons.

Expression of Tumor Marker Proteins Alpha-fetoprotein (AFP) in Tumors Post-treatment

Frozen tissues are imbedded in Histo-prep or other similar tissue embedding medium. Frozen sections (10 micron) are briefly formalin fixed then processed for immunohistochemistry using a kit from R&D system according to manufacturers' protocol. Expression of AFP is detected using polyclonal anti-AFP antisera. Bound antibody is detected using fluorescein conjugated secondary antibodies and visualized by fluorescence microscopy.

Systemic Expression of Interferon Post-treatment

Although the siRNA expressed from the vectors is below the amount that stimulates interferon response, stimulation of interferon expression in tissues is assayed as part of the initial toxicology assessment. A sensitive and specific test for interferon expression is quantitative RT-PCR. RNA from tissues is assayed at Day 21 for expression of a panel of interferon-induced genes, including, but not limited to, STAT1, IL6, IFNb1, and OAS1. Briefly, following reverse transcription, cDNA for the interferon inducible genes are amplified by PCR and amplification levels detected by SYBR Green. RNA levels are calculated from standard curves as for the qPCR of vector genomic DNA above.

Vector Expressing Multigene Therapeutic Genes

Prior to the in vivo work, test lentiviral vectors expressing therapeutic gene cassettes are tested. The rationale for developing lentiviral vectors that can express multiple therapeutic genes is that a frequent outcome of chemotherapy of cancer is recurrence following emergence of resistance to the therapeutic agent. This is due to the pre-existing heterogeneity in the tumor cell population with respect to sensitivity to an individual agent. The rationale for combination therapy is to use two or more agents with different mechanisms of action to minimize the chance that resistance will develop. This strategy has proven to be effective using conventional chemotherapeutic agents in the clinic.

Combination therapy is applied using the inventive lentiviral vector designs by arranging multiple therapeutic components (p53 suppressor, siRNA towards BCL2, C-MYC, VEGF, and RRM2) into one vector or combining two vectors (each with fewer therapeutic components). Combination of three or more vectors may have better treatment effect than two vectors, but the dosage of each individual vector is difficult to control and potential risk of higher side effect may rise.

The vector combinations that are tested in vitro and in vivo work are listed in Table 2.

TABLE 2
Vector Combinations Tested

| Construct 1 | Construct 2 |
|---|---|
| JZ2 | GXY-CMV-shRNAmir (Bcl-2 + VEGF) |
| JZ2 | GXY-ShRNA (BCL2 + VEGF) |
| JZ2 | GXY-TA-AFP-shRNAmir (Bcl-2 + VEGF + |
| | C-MYC + RRM2) |
| GXY-TA-AFP-p53- | |
| shRNAmir (Bcl-2 + | |
| VEGF + C-MYC + RRM2) | |

Vector Construction Strategy

The design strategy for the vectors is summarized below.

Therapeutic Gene Cassettes

The expression cassettes that are engineered into the virus vectors follow three themes: (1) expression of protein-coding mRNAs that direct synthesis of tumor suppressors; (2) expression of small RNA interference (siRNA) molecules that interfere with expression of proteins required for tumor cell viability or proliferation and tumor development (VEGF); and (3) combination of the tumor suppressors and siRNAs.

Restoration of p53 is performed by expression of protein-coding mRNA. Downregulation of BCL2, C-MYC, VEGF, and RRM2 is carried out using RNA interference (RNAi). The small interfering RNAs (siRNAs) can be derived from several sources, including exogenous synthetic RNA oligos, small hairpin RNAs (shRNAs) that are processed in the cell to siRNA, and microRNA (miRNA) cassette that are also processed in the cell to siRNA.

The cassette that expresses p53 tumor suppressor gene in the vector JZ2 consists of CMVie promoter and p53 coding gene. The cytomegalovirus immediate early gene (CMVie) promoter directs high level transcription of genes in many cell types. The JZ2 virus effectively inhibits the proliferation of HepG2 cell line in vitro .

A single siRNA vector against BCL2 (termed JZ3) was constructed and tested in vitro. The JZ3 virus inhibited the proliferation of HCC cell line HepG2 compared to untreated control.

Expression of multiple siRNA in hepatocellular carcinoma gene therapy applications relies on several types of promoter systems. First is the RNA polymerase III (pol III) promoter H1 which is a natural promoter for small RNA transcription. It drives high levels of short hairpin RNA which are processed intracellularly to mature siRNA. This configuration results in the most efficient suppression of gene expression. However, such high levels of shRNA may saturate and induce innate immune response against double strand RNA. Finally, like CMV promoter, H1 drives expression of siRNA in many types of cells, which means that cancer-specific expression cannot be achieved with this promoter.

Second promoter is CMV that can drive siRNA embedded in miRNA cassette (shRNAmir). The shRNAmir is processed intracellularly to mature siRNA. The efficiency of suppression of gene expression is comparable to the H1 driven system. The risk of triggering innate immune response is also reduced. However, this system cannot achieve cancer-specific expression.

To obtain HCC-specific expression, the alphafetoprotein (AFP) promoter is used. The AFP promoter is relatively weak. To address this issue, the Transcriptional Amplification system (Iyer et al. Two-step transcriptional amplification as a method for imaging reporter gene expression using weak promoters. Proc Natl Acad Sci USA. 2001; 98(25):14595-14600, the contents of which are incorporated by reference herein in their entirety for the disclosure of the TA system) is used in conjunction with the AFP promoter. This system uses the AFP promoter to direct synthesis of the strong transcriptional activator, Ga14-VP16, which in turn drives synthesis of the tumor suppressor protein via its cognate promoter, achieving high levels of expression. This system has been applied successfully to the treatment of prostate tumor by combining with prostate-specific promoter hCCKAR. The TA and AFP are assembled onto lentiviral vector.

The three systems described above are compared for therapeutic efficiency (HCC proliferation inhibition) and innate immune response (Interferon a response test) in vitro.

p53 suppressor gene and multiple siRNA are assembled into one vector under the control of the TA-AFP promoter system.

Three variations on the viral backbone are used to house expression cassettes to maximize efficiency of expression of therapeutic genes. The three viral backbone variants are described below.

The CMV(JZ) backbone

This viral backbone includes a hybrid RSV-HIV 5′ long terminal repeat (h5′LTR), the HIV-1 Rev Response Element (RRE), central polypurine tract (cPPT), CMVie promoter, a woodchuck hepatitis virus response element (WPRE), and a self-inactivating (SIN) 3′ LTR. The therapeutic component is inserted between the CMVie promoter and the WPRE. This viral backbone is used in the following plasmids: JZ2, JZ3, GXY-CMV-shRNAmir.

The H1 backbone

This viral backbone includes an RSV 5′ LTR, the disabled HIV-1 GAG gene (GAG), the RRE, the cPPT, WPRE, H1 promoter and a SIN-3′ LTR). The siRNA is placed between the H1 and the 3′d LTR. This viral backbone is used in the following plasmids: GXY-H1-BCL2, GXY-H1- cMYC, GXY-H1-VEGF, GXY-H1-RRM2, GXY-H1-ShRNA.

The TA-AFP backbone

This viral backbone includes an RSV 5′ LTR, the RRE, the cPPT, GAL4VP16 fusion gene, AFP promoter, GAL4 binding site (bs), the WPRE, and the SIN-3′ LTR. The therapeutic component is placed between GAL4 bs and WPRE. The backbone is used in the following plasmids: GXY-TA-AFP-Luc, GXY-TA-AFP-shRNAmir, and GXY-TA-AFP-p53-shRNAmir.

Common Plasmid Substrates

Two packaging plasmids are used to provide structural genes required for virion formation in trans. The plasmid pVSVG contains the G env gene from vesicular stomatitis virus (VSV) driven by the CMVie promoter. This plasmid supplies Env protein to pseudotype the virions. The plasmid, pHelp, contains the gag gene, pol gene, tat gene, and rev gene from HIV-1 as a polycistronic gene driven by the CMVie promoter. This plasmid will supply the remaining structural and helper proteins needed for viable virion formation.

The vector plasmids that are constructed as described above are listed in Table 3.

TABLE 3
Vector Plasmid List

| Name | Expression Cassette |
|---|---|
| JZ2 | P53 driven by CMVie promoter |
| JZ3 | Bcl-2 SiRNA driven by CMVie promoter |
| GXY-H1-Bcl2 | Bcl-2 SiRNA driven by H1 promoter |
| GXY-H1-cMYC | cMYC SiRNA driven by H1 promoter |
| GXY-H1-VEGF | VEGF SiRNA driven by H1 promoter |
| GXY-H1-RRM2 | RRM2 SiRNA driven by H1 promoter |
| GXY-CMV- | multiple SiRNA of (BCL2 + VEGF) in |
| shRNAmir | microRNA cassette driven by CMVie promoter |
| GXY-TA-AFP- | multiple SiRNA of (BCL2 + C-MYC + VEGF + |
| shRNAmir | RRM2) in microRNA cassette driven by AFP promoter |
| | under the control of transcription amplification system |
| GXY-H1-ShRNA | multiple shRNA LV (BCL2 + VEGF) driven |
| | by H1 promoter |
| GXY-AFP-Luc | luciferase reporter gene driven by AFP promoter |
| GXY-TA-AFP- | luciferase reporter gene driven by AFP promoter under |
| Luc | the control of transcription amplification system |
| GXY-TA-AFP- | p53 gene, and multiple SiRNA of (BCL2 + C-MYC + |
| p53-shRNAmir | VEGF + RRM2) in microRNA cassette driven by AFP |
| | promoter under the control of transcription |
| | amplification system |

Methods used: Standard molecular cloning methods are used for construction of these plasmids. Briefly, one-step cloning in which the vector is linearized with the appropriate restriction enzyme, then ligated to insert fragment generated by PCR or restriction digests. Desired recombinants are identified by miniprep, PCR/gel electrophoresis screens, and DNA sequencing. In certain cases, construction of intermediate vector plasmids are required to produce the appropriate components of the therapeutic genes or the viral backbone. Similar construction procedures are followed for these intermediate plasmids.

The shRNAmir cassette contains four siRNA. It is designed with the miRNA 17-92 gene frame, HCC specificity, and driven by the TA-AFP system.

The TA-AFP system is tested as follows. A luciferase reporter gene is cloned downstream of the promoter system. The reporter system is transfected separately into HepG2 cells and 293T cells and checked to see if luciferase activity is only detectable in HepG2, not in 293T, the non HCC cell line. In a separate transfection experiment, the luciferase level driven by TA-AFP system is compared to that driven by CMVie promoter to ascertain the comparative expression level.

JZ2 (expresses p53 gene) and JZ3 (expresses Bcl-2 siRNA) vectors can be effectively packaged into lentiviral particles and kills HepG2 cells following infection in vitro. Furthermore, JZ2 and JZ3 vectors lead to nearly complete tumor regression over the course of 4 weeks when the therapeutic virus was injected into PC3 prostate cancer tumors in immunodeficient mice. Immunostaining of human prostate specific antigen (hPSA) in residual tissue mass at the tumor site was negative, while hPSA was positive in the untreated PC3 tumor sections.

Gene Expression

Expression of the siRNA constructs are assessed by knockdown of the cognate target in HepG2 cells or other appropriate cell types as described. The knockdown efficacy of multiple siRNA vectors for each target gene are compared to single siRNA vector to ensure each siRNA component in multiple-siRNA construct is fully functional and comparable to single-siRNA construct. Briefly, cells are infected at a high multiplicity of infection (moi) and incubated for 2-4 days to allow integration of virus and expression of the therapeutic gene cassette.

For analysis of target gene expression by RT-PCR, RNA is harvested from test and control cultures using Trizol reagent (Invitrogen). A qPCR assay is used to quantitate the level of target gene expression. The percentage decrease in target gene expression relative to vector control is calculated.

Expression of target gene protein is also measured to assess function of the siRNA constructs. Test and control cultures are established as described above. Cells are lysed in sample buffer, subjected to SDS-PAGE, transferred to nylon membranes, and probed with antibody to target protein conjugated to Luc as described.

Interferon a Test for siRNA Virus

Exogenous siRNA may trigger innate immune response, which will cause translation inhibition or apoptosis of infected cells. Interferon a accumulated in cell culture medium after siRNA virus infection is a good indicator of such adverse effect. Interferon a is assayed for using an ELISA method. The siRNA viruses that elicit undetectable or low level of Interferon a response are chosen for further study.

The contents of all cited references, including literature references, issued patents, published patent applications, and co-pending patent applications, cited throughout this application are hereby expressly incorporated by reference in their entirety.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Vector construction

The additional components of the gene transfer system include a packaging (helper) plasmid and an envelope (Env) plasmid encoding VSV-G driven by the HIV-1 LTR (Mochizuki, H. et al., 1998, J Virol 72:8873-8883; Reiser, J. et al., 1996, PNAS USA 93:15266-15271). These packaging and envelope constructs are described herein and by Lai et al., 2000, PNAS, 97: 11297-11302, and Lai, et al., 2002, Neurosci. Res. 67: 363-371, incorporated herein by reference. The helper construct has a deletion in the packaging signal rendering it inactive, and the 5′ LTR is replaced with the CMV-IE promoter. The CMV promoter was derived from pcDNA 3.1, which was used as a template for a PCR reaction yielding 590 bp of the CMV promoter. The HIV-1 helper construct was digested by EcoRV (33) and Afl II (517) to have generate a deletion of 475 bp from the U3 region of the 5′LTR. Insertion of the CMV promoter PCR fragment was then ligated into the HIV-1 helper construct to create a safer Tat-independent construct without compromising viral titer. The viral genes tat and nef were also inactivated. Pseudotyped vectors were produced in human embryonic kidney 293T cells using a three-component transient packaging system (Mochizuki, H. et al., 1998).

The transfer vectors were based on HIV-1 lentivirus vectors, and were made using methods similar to those described by Kim, et al., J. Gene Med. (2005), referenced above. The transfer vectors of the present invention are self-inactivating, i.e., they have a deletion in the U3 region of the 5′ LTR that was introduced as follows: (a) the fragment between Nef and the 3′ LTR was isolated by digesting with Xho-I and Afl-II; (b) the isolated fragment of about 820 bp was subcloned into the PUC18 vector; (c) the U3 region (containing the TATA box, SP1) of the 3′ LTR of about 337 bp was deleted by EcoR-V and Rsa-I; and (d) a PCR fragment from (c) containing the regions of Nef and the 3′ LTR with the U3 deletion were ligated back into the lentiviral vector.

### Example 2

### In Vitro Infection of a Prostate Cancer Cell Line with SIN-HIV-P53-EGFP

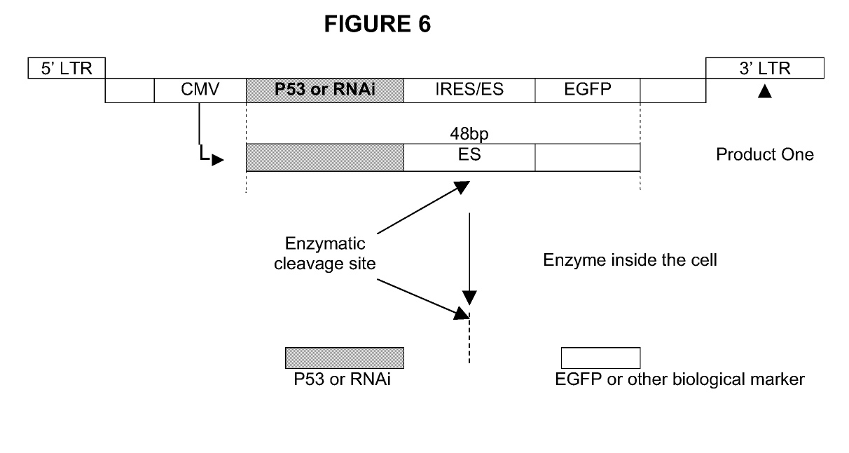

PC-3 (prostate cancer) cells were infected with a lentivirus containing a gene transfer vector expressing wild-type P53 and EGFP. Three groups were tested. Treatment group: PC-3 cells were treated with SIN-HIV-P53-EGFP (construct shown in FIG. 6). Control (negative) group: PC-3 cells were untreated. Control (positive) group: Normal cells were treated or not treated with SIN-HIV-P53-EGFP.

Prostate cancer cell-lines (PC-3) and 293-T cells were placed into the 12 well-plate with 0.4×10⁶ cells, and were cultured in the RPMI 1640 and DMEM medium containing antibiotics. 200 μl of the packaged lentiviral vector SIN-CMV-p53-IRES/CS-EGFP (see FIG. 1C) was added to the cells for the infection study under 1 mg/ml. The cells were then cultured by incubating for 6-16 hours at 37° C. in a CO₂ incubator. After infection for 48-72 hours, we evaluated the expression of EGFP using a fluorescence microscope in both the transduced PC-3 and 239-T cells. We found that a high level of EGFP was expressed in both PC-3 and 293-T cells transduced by SIN-CMV-p53-IRES/CS-EGFP, but not in the control group of those cells that were not infected (FIGS. 8A-C). The results indicated that the vector system worked well to express both transgenes simultaneously.

After treatment, RNA was isolated from the cells, and RT-PCR was used to determine the p53 expression level at different time points. Corresponding cell growth curves and survival cell numbers were determined at different time points, and the results for treated and untreated cells compared.

To evaluate confirm P53 expression, we extracted total RNA 96 hours after infection. 0.5 μg total RNA from each group (treated and untreated) were used as template for RT-PCR. P53 mRNA was amplified using sense primer (from the partial promoter of CMV: 5′-tacgtattagtcatcgctatt-3) and antisense primer (from the end of the P53 gene: 5′-aggcctcattcagctctcgga-3′). The results showed that the cell lines infected by the vector expressed a high level of P53, and the uninfected cell lines expressed only the basic level of endogenous p53 (see FIG. 8D). Thus, the data indicated that the vector system is functional for highly expressing both the two transgene protein in the cells at the same time.

We also compared growth and survival of the prostate cancer cells transduced with the vector with untreated cells. Taking time points for up to one week, we observed the growth condition and cell numbers under the light and fluorescent microscopes. Expression of EGFP indicated transduction by the vector. We observed that PC-3 cells transduced by the vector died quickly compared with the untreated PC-3 cells, and that both treated and untreated 293-T cells showed growth and no significant cell death. The result can be confirmed using FACS analysis to quantitate cell numbers at the respective time points.

### Example 3

### In Vivo Transduction with SIN-HIV-P53-EGFP and SIN-HIV-Bcl-2 RNAi-EGFP

Transduction with the SIN-HIV-P53-EGFP vector described in Example II, expressing P53 and GFP, or SIN-HIVBcl-2 RNAi-EGFP , expressing EGFP and an RNAi agent targeting human Bcl-2, is done in vivo. The NOD SCID mouse model, characterized by a major immunodeficiency, is used to study gene therapy for prostate cancer using lentiviral transfer systems of the invention.

NOD SCID mice are subcutaneously implanted with a PC-3 cell suspension in a thoracic postero-lateral wound. Tumor cell suspensions are injected using a 30-gauge needle and a 1-ml disposable syringe. The volume of inoculation is 100 μl (2×10⁶ tumor cells suspended in 100 μl of PBS). After tumor appearance (1-2 weeks post-implantation), the virus injections are made.

Tumor progression is monitored by palpation twice a week by the investigator, and subcutaneous tumor size is measured using a caliper. Viral vector is administered at a titer of about 10⁸ pfu/ml, by tail-vein injection. Animals are euthanized according to tumor size or clinical status during the observation period. Cervical dislocation is performed 2 to 4 weeks after injection.

Prostate cancer tumor sizes are measured, and for EGFP immunostaining to evaluate distribution of the vector, liver, lung, heart, and bone marrow cells are harvested. These tissue samples are collected for immunostaining does as described by Lai, et al., PNAS, 2002. The tissues are subjected to total RNA and protein extractions, to evaluate expression of the transgenes. The tissue slides are made and examined for EGFP fluorescence under the fluorescence microscope, to determine the distribution of the vector and thereby identify target tissues in the animal model. P53 expression is evaluated by RT-PCR of tissue observed to express EGFP under the fluorescent microscope. The function of the RNAi agent targeted to bcl-2 is be tested by both RT-PCR of RNA and Western-blot protein analysis in the same samples, to determine whether the level of Bcl-2 in tumor tissue is significantly downregulated in the same target cells that express EGFP.

Co-localization of the target cells expressing the P53 or RNAi and EGFP simultaneously is evaluated, particularly those cells in tumors that underwent size reduction as a result of vector treatment. The distribution, e.g., in bone marrow, of the vector after i. v. injection is also determined, to provide information useful for human clinical trials using the vector coexpressing P53 and a bcl-2 RNAi agent.

### Example 4

### In vitro effects of combination of SIN-HIV-P53 and SIN-HIV-Bcl-2 RNAi on prostate cancer cells

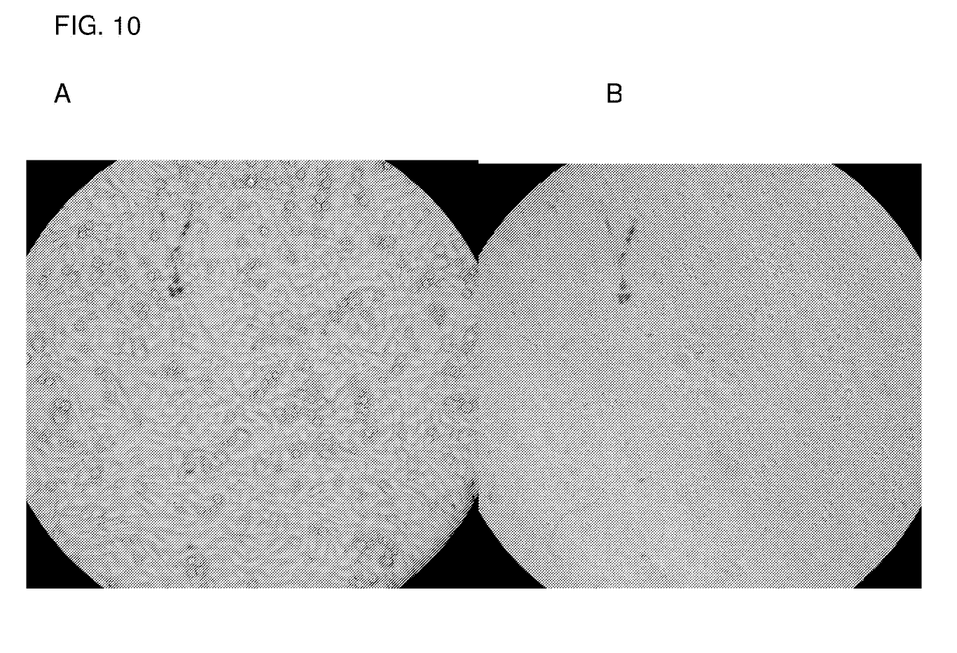

PC3 prostate cancer cells grown on a substrate were contacted with combination of two viral vectors that express P53 and Bcl-2 RNAi, respectively, which is discussed above. FIGS. 10A and 10B show in vitro test results that show that two (P53 and Bcl-2 RNAi) viral vectors induces cell necrosis of PC3 prostate cancer cells. In living culture, FIG. 10A shows the untreated cells, and the FIG. 10B shows cells treated with the double gene vector.

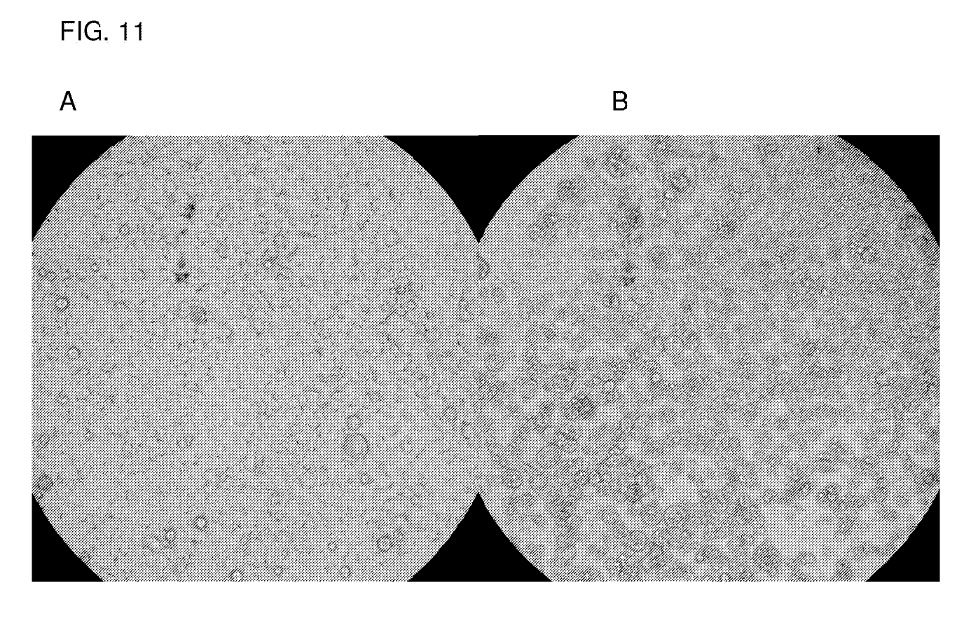

However, when tested on human embryonic kidney cells (293T), two viruses did not cause necrosis. FIG. 11A shows the untreated cells and FIG. 11B shows cells treated with the two viruses, expressing P53 and an RNAi agent targeting human Bcl-2. No difference in necrosis is observed, indicating specificity of the combination of the two viruses for prostate tumor. The cells were observed three days after infection.

### Example 5

### In vivo effect of combination of SIN-HIV-P53 and SIN-HIV-Bcl-2 RNAi over SIN-HIV-P53 and SIN-HIV-Bcl RNAi alone on prostate tumor-first study

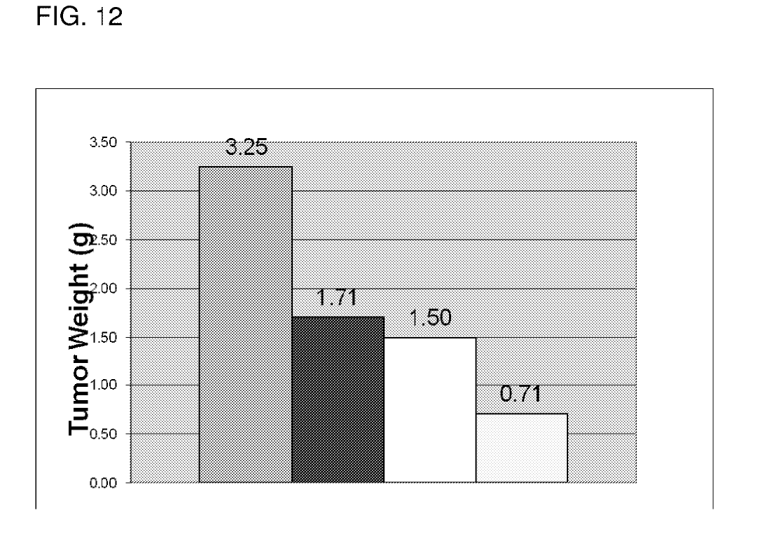

In vivo effects of the combination of the two viruses and the individual viruses were compared against prostate tumor. The results show that tumor reducing effects were seen with the combination of the viruses expressing P53 and RNAi targeting human Bcl-2. FIG. 12 is a graph showing this effect. In order from left to right, far left bar is untreated control group; P53 alone (repair); Bcl-2 siRNA alone (downregulation of BCL2 gene); and on the far right combination of the two viruses. Whereas application of the P53 expressing virus alone reduced the tumor weight to 1.71 grams, and 1.5 grams when only Bcl-2 siRNA expressing virus was applied, application of the combination of the two viruses resulted in a tumor weight of 0.71 grams.

In the in vivo studies, 100 μl of viruses (5.0×10⁸ cells) were injected into the tail vein of mice for three weeks. After injection for three weeks, the mice were sacrificed and tumors were harvested and weighed.

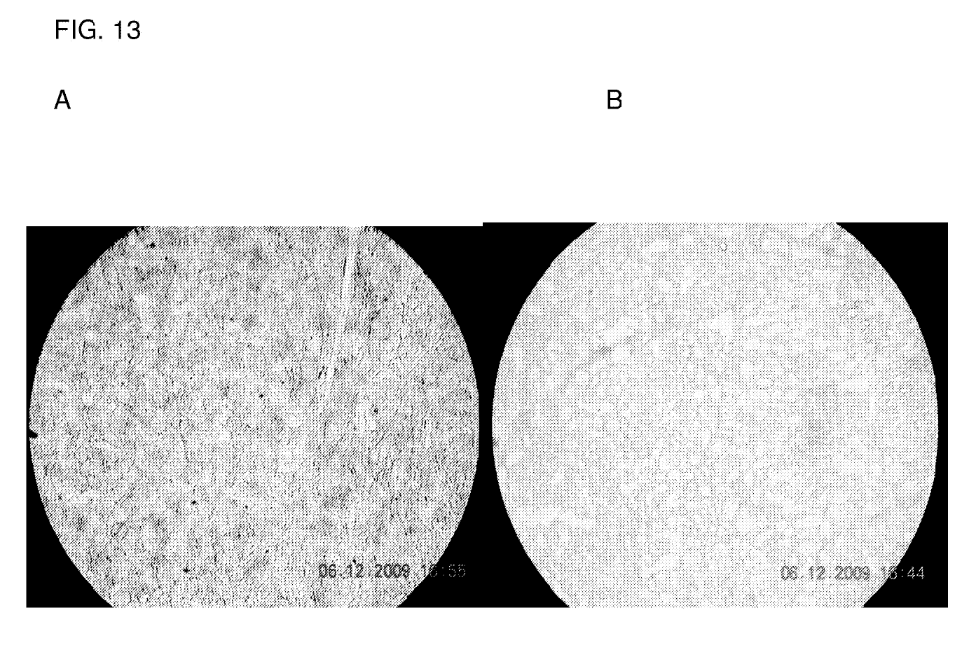

Further, cells obtained from the untreated tumor and tumor treated with the virus combination were examined for human prostate-specific antigen (hPSA) expression. FIGS. 13A and 13B show staining of control (untreated tumor) versus the treatment group. The results show that tumor cells treated with the virus combination no longer produce hPSA, which means that the cells are no longer active tumor cells or are no longer actively cancerous. After human prostate specific antibody staining, FIG. 13A shows the control tumor, which expresses hPSA, and FIG. 13B shows treatment tumor that shows cell necrosis and no expression of hPSA.

### Example 6

### In vivo effect of combination of SIN-HIV-P53 and SIN-HIV-Bcl-2 RNAi over SIN-HIV-P53 and SIN-HIV-Bcl RNAi alone-second study

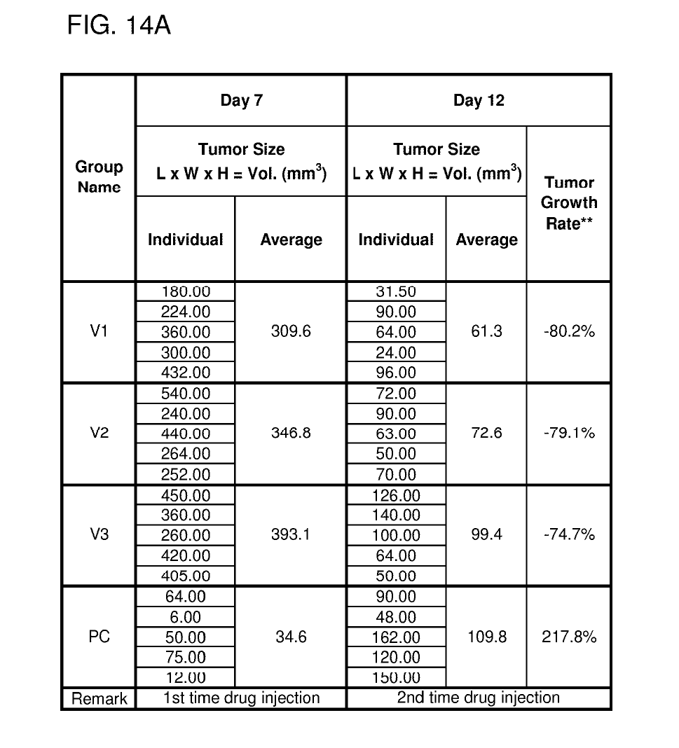

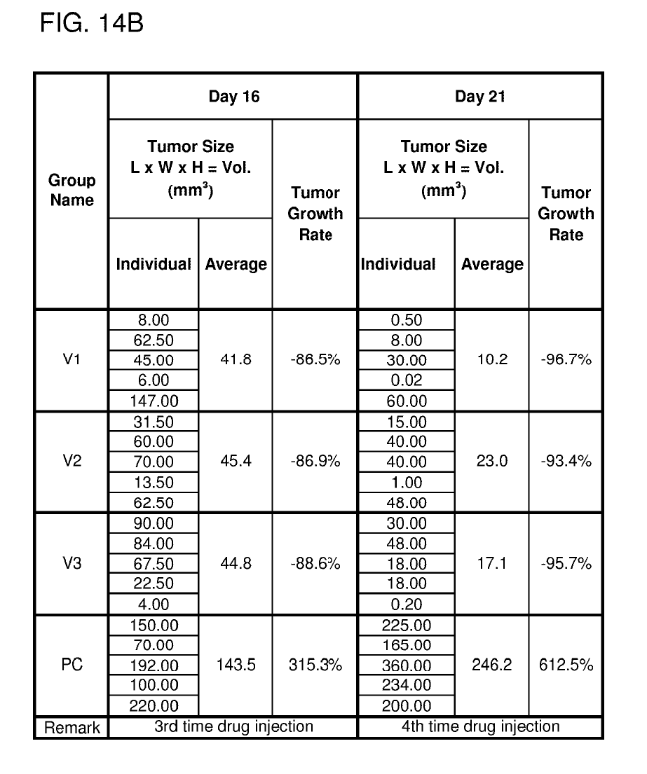

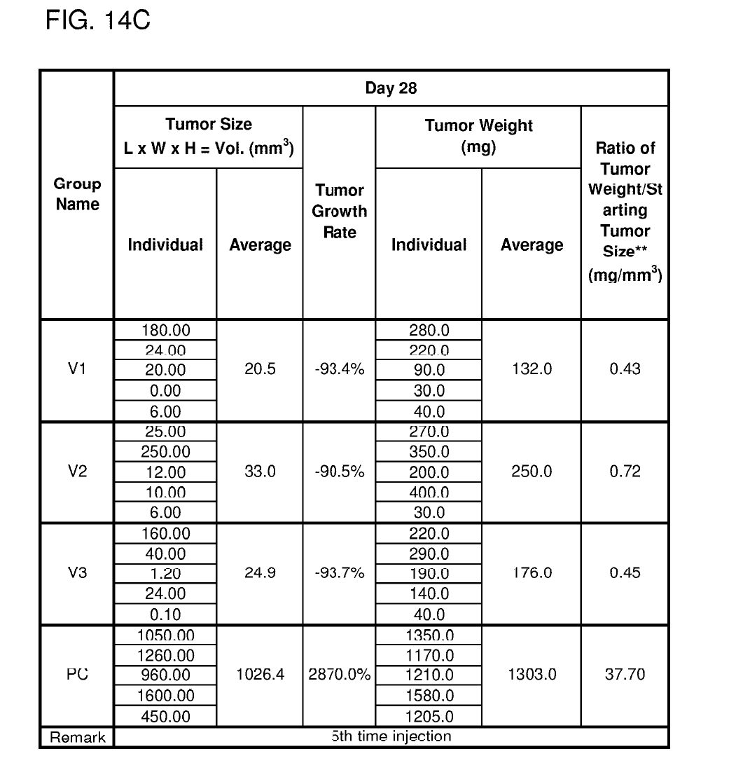

A second study was carried out using mouse models for prostate cancer using the individual viruses for P53 and Bcl-2 RNAi, and the combination of the two viruses. Multiple mice were tested. V1 Group is mice treated with P53 expressing virus alone, V2 Group is mice treated with Bcl-2 RNAi expressing virus alone, and V3 Group is mice treated with the combination of the two viruses. PC is the untreated control mice. As shown in the table in FIGS. 14A-14C, the mice were injected with the viruses and their tumor size measured. Injection days were Days 0, 5, 9, 14 and 21. Tumor size measurement days were Days 7, 12, 16, 21 and 28. Comparison of the tumor size of the V1, V2, V3 mice show that the tumor size in these mice were much reduced compared with the tumor size in the control PC mice. Comparison of the tumor weight on Day 28 in FIG. 14C indicates that V1, V2, V3 mice tumor weight were much reduced compared with the tumor weight in the control PC mice. The ratio of tumor weight/starting tumor size at day 7 shows a large difference in the V1, V2 and V3 Groups compared with that of the PC control mice.

V3 Group was injected with half of the dosage concentration compared with V1 and V2 Groups. Yet its tumor size is smaller than V2 and close to V1.

### Example 7

### Treatment of Human Patients with SIN-HIV-CMV-p53-hPSA-RNAi-huCD25

To investigate the combination gene therapy effects of the gene vector for treatment of human metastatic prostate cancer, adult bone marrow cells (BMC) genetically modified by transduction with SIN-HIV-CMV-p53-hPSA-RNAi-huCD25 (FIG. 1C, Panel C, lower construct) are administered to patients with metastasized prostate cancer by the following procedure: (1) blood is harvested from the patient to collect stem cells. Recent medical advances now make it possible to collect stem cells from circulating blood as well. The collection or harvesting of bone marrow is typically done in a hospital operating room under general anesthesia. The bone marrow is then frozen and stored until gene therapy is completed. (2) The bone marrow cells are transduced ex vivo by the gene transfer vector expressing P53, RNAi and huCD25. To select only those cells stems transduced by the transfer vector, cells expressing the selectable marker huCD25 are selected. Stem cell selection is one method used for purging tumor cells. Recent clinical studies have demonstrated that stem cell selection reduces the tumor contamination found in mobilized blood. This is possibly because stem cells have unique properties not shared by tumor cells. (3) After evaluation of the transduced stem cells, and marker selection, the stem cells are thawed and returned to the patient. This procedure is often referred to as the transplant. Within a few days after completing the gene therapy, the stored stem cells are transplanted, or re-infused into the patient's bloodstream. The re-infusion process is similar to a blood transfusion and takes place in the patient's room: it is not a surgical procedure. The frozen bags of bone marrow or blood cells are thawed in a warm water bath, and then injected into the bloodstream through the catheter. It usually takes 2-4 hours for the infusion. Infused stem cells travel through the bloodstream, and eventually, to the bone marrow where they begin to produce new white blood cells, red blood cells, and platelets. (3) The patients are evaluated by collection of their blood to determine the level of P53, Bcl-2, and the selectable marker (i.e., huCD25) at different time points following treatment using Real Time PCR. At the same time a routine diagnosis of index is done to see if any indexes of cancer markers disappear in the current clinical settings.

### Example 8

### Treatment of Liver Cancer in Human Patients with SIN-HIV-CMV(gp)-P53-hAFP(sp)-hAEG-1 RNAi

Hepatocellular carcinoma (HCC, also called malignant hepatoma) is a primary malignancy (cancer) of the liver. Most cases of HCC are secondary to either a viral hepatitide infection (hepatitis B or C) or cirrhosis (alcoholism being the most common cause of hepatic cirrhosis). Treatment for HCC is often ineffective. Recently approved chemotherapy drug to treat metastic liver cancer provides disease stabilization but not a cure.

Gene therapy provides “root treatment” of liver cancer. Individualized multimodal therapy for liver cancer patients is created. In liver cancer, there is significant up-regulation of the alpha fetoprotein (AFP) gene in HCC. The AFP promoter, which is a cancerous liver tissue cell specific promoter, has been shown to confer selective and specific expression of a transgene in vitro and in vivo in liver cancer (Kanai et al., Cancer Research 57(3) 461-465, 1997).

In one aspect, a multi-gene vector is constructed that includes a general promoter such as CMV-promoter linked to a first gene encoding an anti-tumor protein, such as p53 gene, and further for separate expression in for example a separate secondary expression unit or cassette, a tumor specific promoter placed upstream of a second gene encoding a human anti-tumor protein. However, this second “gene” may encode an anti-sense RNA or RNAi that downregulates a gene product that is overexpressed in the liver tumor cell. Such a liver cancer specific promoter may be the hAFP gene promoter, which is placed upstream of an a sequence encoding the antisense RNA or RNAi specific for down regulating astrocyte elevated gene-1 (AEG-1). P53 is an anti-tumor protein, and AEG-1 is over-expressed in liver tumor. Using this construct, the first P53 protein is expressed in normal and cancerous cells because its expression is controlled by a general promoter. On the other hand, because expression of the second gene component RNAi or anti-sense RNA specific to AEG-1 protein is regulated by a liver tumor specific promoter such as hAFP gene promoter, the antisense RNA and RNAi targeted to AEG-1 is expressed only in the liver cancer cell. (gp) next to the gene designation indicates the general promoter nature of the promoter for the designated gene. (sp) next to the gene designation indicates the tissue specific promoter nature of the promoter for the designated gene.

### Example 9

### Treatment of Liver Cancer in Human Patients with SIN-HIV-hAFP(sp)-p53-TIS-hAEG-1 RNAi

Another vector is constructed in which genes encoding anti-tumor proteins, such as P53 and anti-sense RNA or RNAi targeted to hAEG-1, are both independently under the control of liver tumor tissue-specific promoters, in particular as exemplified, the hAFP gene promoter. The first and second genes are separated by a translation initiation sequence (TIS) such as IRES. Since both of these genetic elements are under the control of liver tumor cell specific promoters, the genetic elements are expressed only in the liver tumor cell.

### Example 10

### In vitro effects of combination of SIN-HIV-P53 and SIN-HIV-Bcl-2 RNAi on liver cancer and normal liver cell lines

Comparative therapeutic effects on cell viability in vitro are assessed using an MTS assay. Briefly, HepG2, or Hep3B cells are seeded in 24 well plates. Serial 2-fold dilutions of test and control virus stocks are used to infect cells. Following incubation at 37° C. for 72 hours cell viability are measure using MTS metabolic assay. A normal liver cell line FL62891 are treated in parallel. The optimal viral titer are defined as at that achieves high level inhibition of HCC cell while does not obviously affect viability of normal cell line. This optimal titer are used to guide dosing in animal studies.

### Example 11

### In Vitro Studies on Viruses Based on the JZ Backbone

The JZ2 vector and the JZ3 vector have been constructed. JZ2 expresses the wild type human p53 gene and the eGFP gene, from a polycistronic expression cassette; JZ3 expresses a human BCL2 siRNA and the eGFP gene from a polycistronic expression cassette.

Function of both was verified by transfection and subsequently infection of HEK-293T cells, followed by fluorescence microscopy. For example, JZ2 was cotransfected with pVSVG and pHelp into HEK-293T cells. Plates of transfected cells were incubated overnight at 37° C., then one sample was processed for fluorescence microscopy. Expression of the eGFP protein was readily detected.

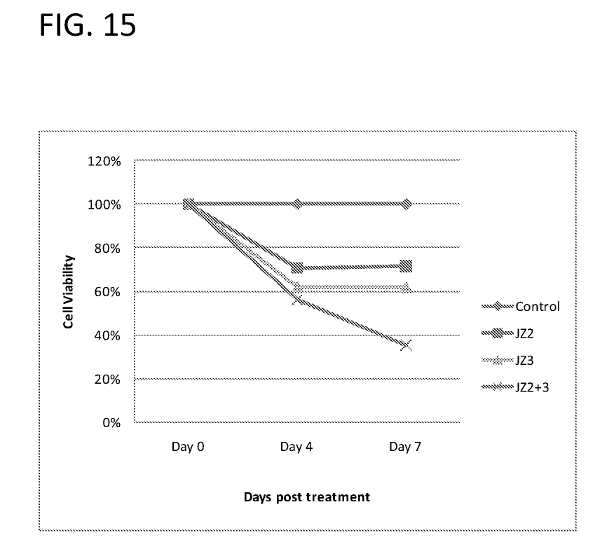

The activity of the JZ viruses on cell growth was tested in HepG2 cells in vitro. HepG2 cells were plated in 96 well plates at 7×10³ cells per well, then infected with either JZ2 virus, JZ3 virus, or a mixture of JZ2 and JZ3 virus. After 8 hr incubation in the presence of the virus, the medium was changed. Cell viability was measured using the MTS assay at Day 4 and Day 7. The relative cell viability data are shown graphically in FIG. 15.

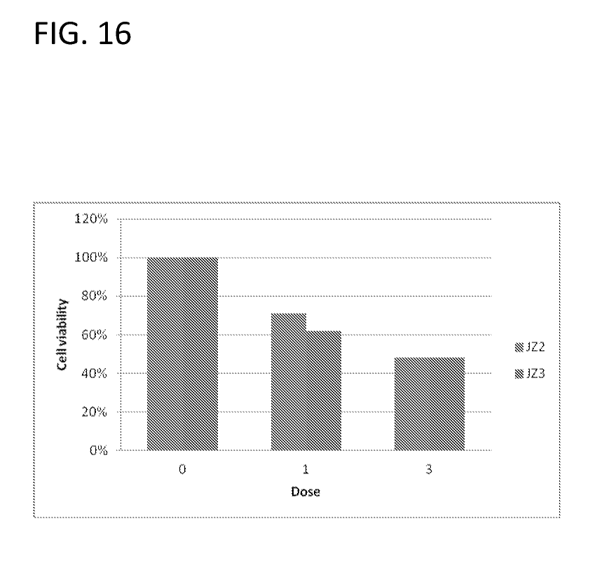

The dose response of the JZ viruses on cell growth was tested in HepG2 cells in vitro. HepG2 cells were plated in 96 well plates at 7×10³ cells per well, then infected with different dose of JZ2 or JZ3 virus (1 dose is 6.4×10⁸ physical particles for JZ2 and 1.2×10⁹ for JZ3). After 8 hr incubation in the presence of the virus, the medium was changed. Cell viability was measured using the MTS assay at Day 4. The relative cell viability data are shown graphically in FIG. 16.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A lentiviral transfer system comprising:
(i) a self-inactivating transfer vector comprising: multiple gene units, wherein each gene unit comprises a heterologous nucleic acid sequence operably linked to a regulatory nucleic acid sequence, wherein one or more of the regulatory nucleic acid sequence comprises a liver cell or liver tissue-specific promoter; and
(ii) a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional.

2. The lentiviral transfer system of claim 1, wherein said cell or tissue-specific promoter is hAFP promoter or liver-selective phosphoenolpyruvate carboxykinase promoter and optionally comprises Transcription Amplification system.

3. The lentiviral transfer system of claim 1, wherein in the transfer vector, a translation initiation site is located between gene units.

4. The lentiviral transfer system of claim 3, wherein the translation initiation site is internal ribosome entry site (IRES).

5. The lentiviral transfer system of claim 1, wherein the heterologous nucleic acid sequence encodes an RNAi, anti-sense RNA or a polypeptide.

6. The lentiviral transfer system of claim 5, wherein the polypeptide or RNAi inhibits expression or activity of a gene or protein that contributes to progression of liver cancer.

7. The lentiviral transfer system of claim 6, wherein the polypeptide or RNAi inhibits expression or activity of B cell leukemia/lymphoma-2 (Bcl-2), myc oncogene, or astrocyte elevated gene-1 (AEG-1).

8. The lentiviral transfer system of claim 5, wherein the polypeptide, anti-sense RNA or RNAi inhibits expression or activity of a growth factor, growth factor receptor, angiogenic factor, angiogenic factor receptor, cell cycle regulator, apoptosis-inducing molecule, or cell adhesion molecule.

9. The lentiviral transfer system of claim 5, wherein the polypeptide, anti-sense RNA or RNAi inhibits the expression or activity of a vascular endothelial growth factor, a vascular endothelial growth factor receptor, epidermal growth factor receptor, hTR, hTERT, papillomavirus E6, papillomavirus E7, BCR-abl, CEACAM6, MMP9, AEG-1, Bcl-2, C-Myc, M2 subunit of ribonucleotide reductase (RRM2), or a cathepsin.

10. The lentiviral transfer system of claim 5, wherein said anti-sense RNA or RNAi is targeted to Bcl-2.

11. The lentiviral transfer system of claim 5, wherein said polypeptide is P53 protein.

12. The lentiviral transfer system of claim 8, wherein said heterologous nucleic acid sequence encodes the P53 protein and anti-sense RNA or RNAi is targeted to Bcl-2.

13. The lentiviral transfer system of claim 1, comprising a cell trafficking signal.

14. The lentiviral transfer system of claim 13, wherein the trafficking signal is the HIV-Tat eleven amino acid transduction sequence.

15. The lentiviral transfer system of claim 13, wherein said trafficking signal is a VP22 protein homologue of HSV1 VP22.

16. The lentiviral transfer system of claim 15 wherein the VP22 transport signal comprises the C-terminal 34 amino acid sequence of VP22 of HSV1, or a fragment having 80% or greater identity to the terminal 34 amino acid sequence of VP22 of HSV1.

17. The lentiviral transfer system of claim 15, wherein the VP22 transport signal comprises one or more of RSASR, RTASR, RSRAR, RTRAR, ATATR, or RSAASR.

18. A method for treating liver cancer, comprising administering to a patient a lentiviral particle for gene transfer, said lentiviral particle produced using a lentiviral transfer system comprising:
(i) a transfer vector comprising: multiple gene units, wherein each gene unit comprises a heterologous nucleic acid sequence operably linked to a regulatory nucleic acid sequence, wherein one or more of the regulatory nucleic acid sequence comprises a liver cell or liver tissue-specific promoter; and
(ii) a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional.

19. The method of claim 1, wherein said cell or tissue-specific promoter is hAFP promoter or liver-selective phosphoenolpyruvate carboxykinase promoter and optionally comprises Transcription Amplification system.

20. The method of claim 18, wherein in the transfer vector, a translation initiation site is located between gene units.

21. The method of claim 18, wherein the translation initiation site is internal ribosome entry site (IRES).

22. The method of claim 18, wherein the heterologous nucleic acid sequence encodes an RNAi, anti-sense RNA or a polypeptide.

23. The method of claim 22, wherein the polypeptide or RNAi inhibits expression or activity of a gene or protein that contributes to progression of liver cancer.

24. The method of claim 23, wherein the polypeptide or RNAi inhibits expression or activity of B cell leukemia/lymphoma-2 (Bcl-2), myc oncogene, or astrocyte elevated gene-1 (AEG-1).

25. A pharmaceutical composition comprising a lentiviral particle for gene transfer, said lentiviral particle produced using a lentiviral transfer system comprising:
(i) a self-inactivating transfer vector comprising: multiple gene units, wherein each gene unit comprises a heterologous nucleic acid sequence operably linked to a regulatory nucleic acid sequence, wherein one or more of the regulatory nucleic acid sequence comprises a liver cell or liver tissue-specific promoter; and
(ii) a helper construct which lacks a 5′ LTR, wherein said 5′ LTR has been replaced with a heterologous promoter, said helper construct further comprising: a lentiviral env nucleic acid sequence containing a deletion, wherein said deleted env nucleic acid sequence does not produce functional env protein; a packaging signal containing a deletion, wherein said deleted packaging signal is nonfunctional.

26. The pharmaceutical composition according to claim 25, wherein the heterologous nucleic acid sequence encodes a P53 protein and an antisense RNA or RNAi is targeted for B cell leukemia/lymphoma-2 (Bcl-2), myc oncogene, or astrocyte elevated gene-1 (AEG-1).

27. The pharmaceutical composition of claim 1, wherein said cell or tissue-specific promoter is hAFP promoter or liver-selective phosphoenolpyruvate carboxykinase promoter and optionally comprises Transcription Amplification system.

28. The pharmaceutical composition of claim 25, further comprising a chemotherapeutic agent or a steroid agent.

29. The pharmaceutical composition of claim 28, wherein the steroid agent is prednisolone, cortisone, corticosterone, or dexamethasone.

30. A pharmaceutical composition comprising a self inactivating lentiviral transfer vector comprising multiple gene units, wherein each gene unit comprises a heterologous nucleic acid sequence operably linked to a regulatory nucleic acid sequence.

31. The lentiviral transfer system of claim 1, wherein the transfer vector further comprises mammalian insulator sequence and splice acceptor and splice donor sites, and is free of wPRE (wood-chuck hepatitis virus post-transcriptional element) downstream of a cloning site.


SEQUENCE LISTING (14 sequences)

SEQ ID NO: 1 - protein, 5 aa, Artificial Sequence
  transport motif
RSASR

SEQ ID NO: 2 - protein, 5 aa, Artificial Sequence
  transport motif
RTASR

SEQ ID NO: 3 - protein, 5 aa, Artificial Sequence
  transport motif
RSRAR

SEQ ID NO: 4 - protein, 5 aa, Artificial Sequence
  transport motif
RTRAR

SEQ ID NO: 5 - protein, 5 aa, Artificial Sequence
  transport motif
ATATR

SEQ ID NO: 6 - protein, 6 aa, Artificial Sequence
  transport motif
RSAASR

SEQ ID NO: 7 - protein, 9 aa, Artificial Sequence
  tat derived peptide
GRKKRRQRR

SEQ ID NO: 8 - DNA, 33 bp, Artificial Sequence
  nucleic acid encoding tat derived peptide
tatggcaggaagaagcggagacagcgacgaaga

SEQ ID NO: 9 - protein, 11 aa, Artificial Sequence
  tat derived peptide
YGRKKRRQRRR

SEQ ID NO: 10 - protein, 11 aa, Artificial Sequence
  tat derived peptide
YARAAARQARA

SEQ ID NO: 11 - protein, 11 aa, Artificial Sequence
  tat derived peptide
THRLPRRRRRR

SEQ ID NO: 12 - protein, 11 aa, Artificial Sequence
  tat derived peptide
GGRRARRRRRR

SEQ ID NO: 13 - DNA, 24 bp, Artificial Sequence
  Primer
agctagatagtgtcacctaaatgc

SEQ ID NO: 14 - DNA, 15 bp, Artificial Sequence
  Primer
agcatgcctgctatt